SEQUENCE LISTING (36 sequences)

SEQ ID NO: 1 - DNA, 140 bp, synthetic construct
  misc_feature 1..140: note = Human Bocavirus 1
  source 1..140: mol_type = other DNA
gtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccac

SEQ ID NO: 2 - DNA, 200 bp, synthetic construct
  misc_feature 1..200: note = Human Bocavirus 1
  source 1..200: mol_type = other DNA
ttgcttatgcaatcgcgaaactctatatcttttaatgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcataagcaa

SEQ ID NO: 3 - DNA, 8257 bp, synthetic construct
  misc_feature 1..8257: note = Expression construct
  source 1..8257: mol_type = other DNA
gtatacctgcaggctagccacgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcaagcttggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaagtatatgtgacgtggttgtacagacgccatcttggaatccaatatgtctgccggcgattagatcatgcgcgcgcgcagcgcgctgcgcgcagcgcaggcatgactgagccggcagacatattggattccaagatggcgtctgtacaaccacgtgcttaagctgcagactagtgagctcgttaac

SEQ ID NO: 4 - DNA, 1979 bp, synthetic construct
  misc_feature 1..1979: note = Expression construct
  source 1..1979: mol_type = other DNA
gcggccgcggatccgccaccatggcattcaatccgcccgtaatacgcgcattttcacaacccgcctttacgtatgtctttaagtttccgtaccctcaatggaaagagaaagagtggctactgcacgcgttgcttgcccacggcaccgagcagtccatgattcaattacgtaactgtgccccacacccggacgaggatattatccgggacgatcttctaataagtttggaagataggcatttcggggcggtcctgtgtaaagcggtatacatggctactaccacgttgatgtctcacaagcaacgcaatatgttcccaaggtgcgacataatcgttcagtcagagttaggtgaaaaaaatttacattgtcatattatcgttggaggcgaaggcctatcaaagagaaacgctaagagctcttgcgctcagttttacggacttatattagcagaaattatccagcgctgtaagagtttactagccacccgtccgtttgagccggaagaagcggatatatttcatacgttgaagaaagcggagcgcgaggcctggggtggagttactggcggtaacatgcaaatcttacaatacagggaccgtcggggtgacctgcatgcacagactgttgatcccctcagattcttcaaaaattatttgttaccgaagaaccgatgcataagtagttacagcaaacctgatgtctgtactagccctgataactggttcattctggccgaaaaaacgtactcgcatacacttatcaatggattgccgcttcccgagcactatcgaaaaaactatcatgccaccctggataatgaagttatacctggaccacagactatggcgtatggagggagaggcccttgggaacatttacccgaggtgggtgaccagaggcttgccgcaagttccgtgagcactacgtataagccaaacaagaaggagaagctaatgctcaacctcctcgacaagtgtaaggagttgaatcttctagtttatgaggatcttgtagcgaactgcccagagctgctgctcatgctagaaggccaacctggaggtgctcgactcatcgagcaagtactaggaatgcatcacatcaatgtatgctcgaatttcaccgcgctaacgtacctcttccatctgcatccggtgacatcgctggatagtgacaacaaagcgttacagcttttactaattcaagggtacaaccccctggcagtggggcatgctctctgttgtgtgttaaacaaacaatttggtaaacagaacacagtctgtttttacgggccagcatctactgggaaaacaaatatggcaaaagcgattgtgcagggaatccggctatatggctgcgtcaaccatcttaacaagggttttgttttcaatgattgtcgacaacgcctcgtagtctggtgggaggaatgcctaatgcaccaggactgggtggagccagcaaagtgtattcttggcgggaccgaatgtcgtatcgacgtcaagcacagagattctgtcctattgacacaaacgcctgtaataatttcgactaatcacgacatttacgccgtcgtgggagggaattcggtgtctcacgttcacgctgcgcctctcaaagaacgggttattcagctgaattttatgaaacaactcccccaaacttttggtgagataaccgccacagaaatcgctgctctgctacagtggtgctttaatgaatatgactgcaccctgacaggtttcaaacagaagtggaatttggacaagatacctaactcattcccgttgggggtattgtgcccaacacattcccaagatttcacacttcacgaaaatgggtattgcacggactgcgggggctaccttccccactccgctgataattcaatgtataccgatcgggctagcgaaacatccaccggcgacataacgccctccaaatgattcgaatctagagcctgcagtctcgaggcatgcggtacc

SEQ ID NO: 5 - DNA, 18 bp, synthetic construct
  misc_feature 1..18: note = primer
  source 1..18: mol_type = other DNA
gtggacgtgaaagaaacc

SEQ ID NO: 6 - DNA, 20 bp, synthetic construct
  misc_feature 1..20: note = primer
  source 1..20: mol_type = other DNA
ggtcatagctgtttcctgtg

SEQ ID NO: 7 - DNA, 2033 bp, synthetic construct
  misc_feature 1..2033: note = expression construct
  source 1..2033: mol_type = other DNA
attaagcttccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgggtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacgcggccgcatttgtaaaaaaaaaataaataaaaatgatcgagcaggacggcctgcacgctggttctccagctgcttgggtcgagcgtctgttcggttacgactgggctcagcagaccatcggttgctccgacgctgctgtgttccgtctgtccgctcagggtcgtcccgtgctgttcgtcaagaccgacctgtccggtgctctgaacgagctgcaggacgaggctgctcgtctgtcctggctggctaccactggtgtcccttgcgctgctgtcctggacgtggtcactgaggctggtcgtgactggctgctgctgggagaagtgcctggacaggacctgctgtccagccacctggctccagctgagaaggtgtccatcatggctgacgctatgcgtcgtctgcacaccctggaccctgctacctgccccttcgaccaccaagctaagcaccgtatcgagcgtgctcgtacccgtatggaagctggcctggtggaccaggacgacctggacgaagaacaccagggactggcccctgctgagctgttcgctcgtctgaaggctcgtatgcccgacggcgaggacctggtggttactcacggcgacgcttgcctgcccaacatcatggtcgagaacggtcgtttctccggtttcatcgactgcggtcgtctgggtgtcgctgaccgttaccaggatatcgctctggctacccgtgatatcgctgaggaactgggtggcgagtgggctgacagattcctggtgctgtacggtatcgctgctcccgactcccagcgtatcgctttctaccgtctgctggacgagttcttctaagccccttgtaaacgccacaattgtgtttgttgcaaataaacccatgattatttgattaaaattgttgttttctttgttcatagacaatagtgtgttttgcctaaacgggtacc

SEQ ID NO: 8 - DNA, 1958 bp, synthetic construct
  misc_feature 1..1958: note = Expression construct
  source 1..1958: mol_type = other DNA
attaagcttccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgggtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacgcggccgcatttgtaaaaaaaaaataaataaaaatggtgtccaagggcgaggaactgttcaccggtgtcgtgcccatcctggtcgaactggacggcgacgtgaacggtcacaagttctccgtgtctggcgaaggcgagggcgacgctacctacggaaagctgaccctgaagttcatctgcaccaccggcaagctgcccgtgccttggcctaccctggtcaccactctgacctacggtgtccagtgcttctcccgttaccccgaccacatgaagcagcacgatttcttcaagtccgctatgcccgagggttacgtgcaagagcgtaccatcttcttcaaggacgacggcaactacaagacccgtgctgaagtgaagttcgaaggcgacaccctcgtgaaccgtatcgagctgaagggtatcgacttcaaggaagatggaaacatcctgggccacaagctcgagtacaactacaactcccacaacgtgtacatcatggccgacaagcaaaagaacggcatcaaagtgaacttcaagatccgccacaacatcgaggacggttccgtgcagctggctgaccactaccagcagaacacccccatcggcgacggtcctgtgctgctgcctgacaaccactacctgtccacccagtccgctctgtccaaggaccccaacgagaagcgtgaccacatggtgctgctcgagttcgtgaccgctgctggtatcaccctgggcatggacgagctgtacaagtaagccccttgtaaacgccacaattgtgtttgttgcaaataaacccatgattatttgattaaaattgttgttttctttgttcatagacaatagtgtgttttgcctaaacgggtacc

SEQ ID NO: 9 - DNA, 4824 bp, synthetic construct
  misc_feature 1..4824: note = expression construct
  source 1..4824: mol_type = other DNA
atgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 10 - protein, 1588 aa, synthetic construct
  REGION 1..1588: note = expression construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 11 - protein, 19 aa, synthetic construct
  REGION 1..19: note = signal peptide
MQIELSTCFFLCLLRFCFS

SEQ ID NO: 12 - protein, 1438 aa, synthetic construct
  REGION 1..1438: note = expression construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - DNA, 4371 bp, synthetic construct
  misc_feature 1..4371: note = expression construct
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 14 - DNA, 7891 bp, synthetic construct
  misc_feature 1..7891: note = expression construct
  source 1..7891: mol_type = other DNA
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaag

SEQ ID NO: 15 - DNA, 330 bp, synthetic construct
  misc_feature 1..330: note = enhancer
  source 1..330: mol_type = other DNA
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccacc

SEQ ID NO: 16 - DNA, 345 bp, synthetic construct
  misc_feature 1..345: note = promoter
  source 1..345: mol_type = other DNA
gatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctag

SEQ ID NO: 17 - DNA, 1489 bp, synthetic construct
  misc_feature 1..1489: note = intron
  source 1..1489: mol_type = other DNA
tcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaatta

SEQ ID NO: 18 - DNA, 595 bp, synthetic construct
  misc_feature 1..595: note = WPRE
  source 1..595: mol_type = other DNA
tcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctg

SEQ ID NO: 19 - DNA, 211 bp, synthetic construct
  misc_feature 1..211: note = bGHpA
  source 1..211: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 20 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 21 - DNA, 383 bp, synthetic construct
  misc_feature 1..383: note = parvovirus
  source 1..383: mol_type = other DNA
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 22 - DNA, 383 bp, synthetic construct
  misc_feature 1..383: note = parvovirus
  source 1..383: mol_type = other DNA
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 23 - DNA, 125 bp, synthetic construct
  misc_feature 1..125: note = parvovirus
  source 1..125: mol_type = other DNA
ccaaatcagatgccgccggtcgccgccggtaggcgggacttccggtacagcgcgccgctgtaccggaagtcccgcctaccggcggcgaccggcggcatctgatttggtgtcttcttttaaatttt

SEQ ID NO: 24 - DNA, 125 bp, synthetic construct
  misc_feature 1..125: note = parvovirus
  source 1..125: mol_type = other DNA
aaaatttaaaagaagacaccaaatcagatgccgccggtcgccgccggtaggcgggacttccggtacagcggcgcgctgtaccggaagtcccgcctaccggcggcgaccggcggcatctgatttgg

SEQ ID NO: 25 - DNA, 114 bp, synthetic construct
  misc_feature 1..114: note = parvovirus
  source 1..114: mol_type = other DNA
ctcattggagggttcgttcgttcgaacgttcgttcgcatgcgaacgaacgttcgaacgaacgaaccctccaatgagactcaaggacaagaggatattttgcgcgccaggaagtg

SEQ ID NO: 26 - DNA, 114 bp, synthetic construct
  misc_feature 1..114: note = parvovirus
  source 1..114: mol_type = other DNA
cacttcctggcgcgcaaaatatcctcttgtccttgagtctcattggagggttcgttcgttcgaacgttcgttcgcatgcgaacgaacgttcgaacgaacgaaccctccaatgag

SEQ ID NO: 27 - DNA, 408 bp, synthetic construct
  misc_feature 1..408: note = parvovirus
  source 1..408: mol_type = other DNA
ctcattggagggttcgttcgttcgaaccagccaatcaggggagggggaagtgacgcaagttccggtcacatgcttccggtgacgcacatccggtgacgtagttcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgcgaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 28 - DNA, 408 bp, synthetic construct
  misc_feature 1..408: note = parvovirus
  source 1..408: mol_type = other DNA
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgcgaactacgtcaccggatgtgcgtcaccggaagcatgtgaccggaacttgcgtcacttccccctcccctgattggctggttcgaacgaacgaaccctccaatgag

SEQ ID NO: 29 - DNA, 480 bp, synthetic construct
  misc_feature 1..480: note = parvovirus
  source 1..480: mol_type = other DNA
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgctcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 30 - DNA, 481 bp, synthetic construct
  misc_feature 1..481: note = parvovirus
  source 1..481: mol_type = other DNA
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgagcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacggcatgcgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgag

SEQ ID NO: 31 - DNA, 7053 bp, synthetic construct
  source 1..7053: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 32 - DNA, 4317 bp, synthetic construct
  misc_feature 1..4317: note = expression construct
  source 1..4317: mol_type = other DNA
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 33 - DNA, 4335 bp, synthetic construct
  misc_feature 1..4335: note = expression construct
  source 1..4335: mol_type = other DNA
gccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccggcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 34 - DNA, 4824 bp, synthetic construct
  misc_feature 1..4824: note = expression construct
  source 1..4824: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 35 - DNA, 8458 bp, synthetic construct
  source 1..8458: mol_type = other DNA
atcgatggccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccaccttcgatggccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccaccttcgaactagccactagcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaag

SEQ ID NO: 36 - DNA, 1242 bp, Homo sapiens
  source 1..1242: mol_type = other DNA
atcgatggccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccaccttcgatggccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccaccttcgaactagccactagcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacag

(19) United States
(12) Patent Application Publication
(10) Pub. No.: US 2023/0091932 A1
(43) Pub. Date: Mar. 23, 2023

(54) CLOSED-END DNA PRODUCTION WITH INVERTED TERMINAL REPEAT SEQUENCES

(71) Applicant: BIOVERATIV THERAPEUTICS INC., Waltham, MA (US)

(72) Inventors: Ajay Maghodia, Waltham, MA (US); Christian Mueller, Waltham, MA (US); Tongyao Liu, Waltham, MA (US)

(21) Appl. No.: 17/821,356

(22) Filed: Aug. 22, 2022

Related U.S. Application Data
(60) Provisional application No. 63/236,215, filed on Aug. 23, 2021.

(51) Int. Cl. C12N 15/113; C12N 15/86; C07K 14/755; A61K 31/713

(52) U.S. Cl. CPC C12N 15/113; C12N 15/86; C07K 14/755; A61K 31/713; C12N 2750/14143; C12N 2750/14171; C12N 2750/14343; C12N 2750/14371; C12N 2830/42; C12N 2830/50

(57) ABSTRACT

The present disclosure provides nucleic acid molecules comprising a first inverted terminal repeat (ITR), a second ITR, and a genetic cassette encoding a target sequence. In some embodiments, the first ITR and/or the second ITR is an ITR of human bocavirus. Also disclosed are methods of using the nucleic acid molecules in gene therapy applications.

### RELATED APPLICATIONS

This application claims priority to U.S. Provisional Patent Application Ser. No. 63/236,215, filed Aug. 23, 2021, the disclosure of which is hereby incorporated by reference in its entirety.

### REFERENCE TO SEQUENCE LISTING SUBMITTED ELECTRONICALLY

The content of the electronically submitted sequence listing in XML format (Name: 731943_SA9-481_ST26.xml; Size: 117,456 bytes; and Date of Creation: Aug. 22, 2022) is incorporated herein by reference in its entirety.

### BACKGROUND

Gene therapy offers the potential for a lasting means of treating a variety of diseases. In the past, many gene therapy treatments typically relied on the use of viral vectors. There are numerous viral agents that could be selected for this purpose, each with distinct properties that make them more or less suitable for gene therapy. However, the undesired properties of some viral vectors have resulted in clinical safety concerns and limited their therapeutic use.

Adeno-associated virus (AAV) is a common gene therapy vector, but it is not without its drawbacks. The coding sequences of the AAV genome are flanked by inverted terminal repeats (ITRs) which are required for viral replication and packaging, as well as transgene expression. The T-shaped hairpin structures of AAV ITRs are susceptible to binding by host cell proteins which inhibit transgene expression in AAV vectors. There exists a need to provide efficient and persistent expression of target sequences while avoiding the limitations of existing AAV vector technology.

### SUMMARY OF THE DISCLOSURE

Disclosed herein are nucleic acid molecules and uses thereof comprising a first inverted terminal repeat (ITR) and/or a second ITR flanking a genetic cassette comprising a heterologous polynucleotide sequence.

In one aspect, provided herein is a nucleic acid molecule comprising a first ITR and a second ITR flanking a genetic cassette comprising a heterologous polynucleotide sequence, wherein the first ITR and the second ITR are bocavirus ITRs or fragments/derivatives thereof (e.g., human bocavirus 1 ITRs). In another aspect, provided herein is a nucleic acid molecule comprising a first ITR and a second ITR, wherein the first ITR comprises a polynucleotide sequence at least about 75% identical to SEQ ID NO:1, and the second ITR comprises a polynucleotide sequence at least about 75% identical to SEQ ID NO:2.

In some embodiments, the first ITR comprises a polynucleotide sequence at least about 80%, at least about 85%, at least about 90%, at least about 95%, at least about 96%, at least about 97%, at least about 98%, at least about 99% identical to SEQ ID NO:1. In some embodiments, the first ITR comprises the polynucleotide sequence set forth in SEQ ID NO: 1. In some embodiments, the first ITR comprises a polynucleotide sequence at least about 50% identical to SEQ ID NO: 1.

In some embodiments, the second ITR comprises a polynucleotide sequence at least about 80%, at least about 85%, at least about 90%, at least about 95%, at least about 96%, at least about 97%, at least about 98%, at least about 99% identical to SEQ ID NO:2. In some embodiments, the second ITR comprises the polynucleotide sequence set forth in SEQ ID NO: 2. In some embodiments, the first ITR comprises a polynucleotide sequence at least about 50% identical to SEQ ID NO: 2.

In some embodiments, the first ITR comprises the polynucleotide sequence set forth in SEQ ID NO:1, and the second ITR comprises the polynucleotide sequence set forth in SEQ ID NO:2.

In some embodiments, the nucleic acid molecule further comprises a genetic cassette which comprises a heterologous polynucleotide sequence and at least one expression control sequence, such as a promoter, an enhancer, an intron, a transcription termination signal, or a post-transcriptional regulatory element.

In some embodiments, the genetic cassette further comprises a promoter. In some embodiments, the the promoter is a tissue-specific promoter. In some embodiments, the promoter drives expression of the heterologous polynucleotide sequence in an organ, wherein the organ comprises muscle, central nervous system (CNS), ocular, liver, heart, kidney, pancreas, lungs, skin, bladder, urinary tract, spleen, myeloid and lymphoid cell lineages, or any combination thereof. In some embodiments, the promoter drives expression of the heterologous polynucleotide sequence in hepatocytes, epithelial cells, endothelial cells, cardiac muscle cells, skeletal muscle cells, sinusoidal cells, afferent neurons, efferent neurons, interneurons, glial cells, astrocytes, oligodendrocytes, microglia, ependymal cells, lung epithelial cells, Schwann cells, satellite cells, photoreceptor cells, retinal ganglion cells, T cells, B cells, NK cells, macrophages, dendritic cells, or any combination thereof. In some embodiments, the the promoter is positioned 5′ to the heterologous polynucleotide sequence. In some embodiments, the promoter is a mouse transthyretin promoter (mTTR), a native human factor VIII promoter, a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, α1-antitrypsin (AAT), muscle creatine kinase (MCK), myosin heavy chain alpha (αMHC), myoglobin (MB), desmin (DES), SPc5-12, 2R5Sc5-12, dMCK, tMCK, or a phosphoglycerate kinase (PGK) promoter.

In some embodiments, the genetic cassette further comprises an intronic sequence. In some embodiments, the the intronic sequence is positioned 5′ to the heterologous polynucleotide sequence. In some embodiments, the the intronic sequence is positioned 3′ to the promoter. In some embodiments, the the intronic sequence comprises a synthetic intronic sequence.

In some embodiments, the genetic cassette further comprises a post-transcriptional regulatory element. In some embodiments, the regulatory element is positioned 3′ to the heterologous polynucleotide sequence. In some embodiments, the regulatory element comprises a mutated woodchuck hepatitis virus post-transcriptional regulatory element (WPRE), a microRNA binding site, a DNA nuclear targeting sequence, or any combination thereof.

In some embodiments, the genetic cassette further comprises a 3′UTR poly(A) tail sequence. In some embodiments, the 3′UTR poly(A) tail sequence is selected from the group consisting of bGH poly(A), actin poly(A), hemoglobin poly(A), and any combination thereof.

In some embodiments, the genetic cassette further comprises an enhancer sequence. In some embodiments, the enhancer sequence is positioned between the first ITR and the second ITR.

In some embodiments, the nucleic acid molecule comprises from 5′ to 3′: the first ITR, the genetic cassette, and the second ITR, wherein the genetic cassette comprises a tissue-specific promoter sequence, an intronic sequence, the heterologous polynucleotide sequence, a post-transcriptional regulatory element, and a 3′UTR poly(A) tail sequence.

In some embodiments, the genetic cassette comprises from 5′ to 3′: a tissue-specific promoter sequence, an intronic sequence, the heterologous polynucleotide sequence, a post-transcriptional regulatory element, and a 3′UTR poly(A) tail sequence.

In some embodiments, the genetic cassette is a single stranded nucleic acid. In some embodiments, the genetic cassette is a double stranded nucleic acid.

In some embodiments, the heterologous polynucleotide sequence encodes a therapeutic protein.

In some embodiments, the heterologous polynucleotide sequence encodes a clotting factor, a growth factor, a hormone, a cytokine, an antibody, a fragment thereof, or any combination thereof. In some embodiments, the heterologous polynucleotide sequence encodes a clotting factor. In some embodiments, the heterologous polynucleotide sequence encodes a growth factor. In some embodiments, the heterologous polynucleotide sequence encodes a hormone. In some embodiments, the heterologous polynucleotide sequence encodes a cytokine.

In some embodiments, the heterologous polynucleotide sequence encodes a FVIII protein.

In some embodiments, the heterologous polynucleotide sequence encodes dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), GALT, OTC, CMD1A, LAMA2, or any combination thereof.

In some embodiments, the heterologous polynucleotide sequence encodes a microRNA (miRNA). In some embodiments, the miRNA down regulates the expression of a target gene comprising SOD1, HTT, RHO, CD38, or any combination thereof.

In some embodiments, the heterologous polynucleotide sequence encodes a clotting factor, wherein the clotting factor comprises factor I (FI), factor II (FII), factor III (FIII), factor IV (FIV), factor V (FV), factor VI (FVI), factor VII (FVII), factor VIII (FVIII), factor IX (FIX), factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), or any combination thereof.

In some embodiments, the heterologous polynucleotide sequence is codon optimized. In some embodiments, the heterologous polynucleotide sequence is codon optimized for expression in a human.

In some embodiments, the nucleic acid molecule is formulated with a delivery agent. In some embodiments, the delivery agent comprises a lipid nanoparticle. In some embodiments the lipid nanoparticle is ionizable. In some embodiments, the delivery agent comprises liposomes, non-lipid polymeric molecules, endosomes, or any combination thereof.

In some embodiments, the nucleic acid molecule is formulated for intravenous, transdermal, intradermal, subcutaneous, pulmonary, intraneural, intraocular, intrathecal, oral administration, or any combination thereof. In some embodiments, the nucleic acid molecule is formulated for intravenous administration. In some embodiments, the nucleic acid molecule is formulated for administration by in situ injection. In some embodiments, the nucleic acid molecule is formulated for administration by inhalation.

In another aspect, provided herein is a vector comprising a nucleic acid molecule described herein.

In another aspect, provided herein is a host cell comprising a nucleic acid molecule described herein, or a vector described herein. In some embodiments, the host cell is an insect cell.

In another aspect, provided herein is a pharmaceutical composition comprising a nucleic acid molecule described herein.

In another aspect, provided herein is a pharmaceutical composition comprising a vector described herein and a pharmaceutically acceptable excipient.

In another aspect, a pharmaceutical composition is provided herein comprising a host cell described herein and a pharmaceutically acceptable excipient.

In another aspect, a kit is provided herein comprising a nucleic acid molecule described herein and instructions for administering the nucleic acid molecule to a subject in need thereof.

In another aspect, provided herein is a baculovirus system for production of a nucleic acid molecule described herein.

In some embodiments, the nucleic acid molecule is produced in insect cells.

In another aspect, provided herein is a nanoparticle delivery system comprising a nucleic acid molecule described herein.

In another aspect, provided herein is a method of expressing a heterologous polynucleotide sequence in a subject in need thereof, comprising administering to the subject a nucleic acid molecule described herein, a vector described herein, or a pharmaceutical composition described herein.

In another aspect, provided herein is a method of treating a disease or disorder in a subject in need thereof, comprising administering to the subject a nucleic acid molecule described herein, a vector described herein, or a pharmaceutical composition described herein.

In some embodiments, the nucleic acid molecule is administered intravenously, transdermally, intradermally, subcutaneously, orally, pulmonarily, intraneurally, intraocularly, intrathecally, or any combination thereof. In some embodiments, the nucleic acid molecule is administered intravenously. In some embodiments, the the nucleic acid molecule is administered by in situ injection. In some embodiments, the the nucleic acid molecule is administered by inhalation.

In some embodiments, the subject is a mammal. In some embodiments, the subject is a human.

### BRIEF DESCRIPTION OF THE DRAWINGS

FIGS. 1A-1C are schematic representation of approaches used for ceDNA production in the baculovirus system according to one embodiment of the invention. FIG. 1A shows a schematic diagram of One BAC approach, where a single recombinant BEV encoding FVIIIXTEN and Rep genes at different loci was used for infection in Sf9 cells for ceDNA production. FIG. 1B shows a schematic diagram of the Two BAC approach, where Sf9 cells were co-infected with recombinant BEVs encoding FVIIIXTEN and/or Rep genes for ceDNA production. FIG. 1C shows a schematic diagram of a stable cell line approach, where the FVIIIXTEN expression cassette was stably integrated into the Sf9 cell genome and was rescued by infecting recombinant BEV encoding Rep gene for ceDNA production.

FIGS. 2A-2B are schematic representation of human FVIIIXTEN expression construct. FIG. 2A shows schematic linear map of expression construct according to one embodiment of the invention comprising of B-domain deleted (BDD) codon-optimized human Factor VIII (coFVIII) fused with XTEN 144 peptide (FVIIIXTEN) under the regulation of liver-specific modified mouse transthyretin (mTTR) promoter (mTTR482) with enhancer element (A1MB2), hybrid synthetic intron (Chimeric Intron), the Woodchuck Posttranscriptional Regulatory Element (WPRE), and the Bovine Growth Hormone Polyadenylation (bGHpA) signal. The FVIIIXTEN expression cassette is flanked by the human Bocavirus type 1 (HBoV1) wildtype (WT) ITRs. (SEQ ID NO: 1 and SEQ ID NO: 2). FIG. 2B shows a schematic map of the Tn7 transfer vector according to one embodiment of the invention made by inserting the FVIIIXTEN expression cassette (SEQ ID NO: 3) into the pFastBac1 vector (Invitrogen).

FIGS. 3A-3C are schematic representation of Replication (Rep) gene expression constructs according to the embodiments of invention. FIG. 3A shows a schematic linear map of a synthetic DNA encoding Sf-codon-optimized HBoV1 NS1 gene under the AcMNPV polyhedrin promoter followed by the SV40 polyadenylation signal (SV40 PAS). FIG. 3B shows a schematic map of a Tn7 transfer vector according to an embodiment of the invention made by inserting the HBoV1 NS1 synthetic DNA (SEQ ID NO: 4) into the pFastBac1 vector (Invitrogen). FIG. 3C shows a schematic map of a Cre-LoxP donor vector according to an embodiment of the invention made by inserting the HBoV1 NS1 synthetic DNA (SEQ ID NO: 4) into the Cre-LoxP donor vector created as described in Baculovirus Expression System, U.S. Patent Application No. 63/069,073, incorporated herein for reference in its entirety.

FIGS. 4A-4C are schematic representation of Replication (Rep) gene expression constructs according to the embodiments of invention. FIG. 4A shows a schematic linear map of a synthetic DNA encoding Sf-codon-optimized HBoV1 NS1 gene under the AcMNPV immediate-early1 (pIE1) promoter preceded by the AcMNPV transcriptional enhancer hr5 element followed by the SV40 polyadenylation signal (SV40 PAS). FIG. 4B shows a schematic map of a Tn7 transfer vector according to an embodiment of the invention made by inserting the HBoV1 NS1 synthetic DNA (SEQ ID NO: 4) into the pFastBac1 vector (Invitrogen). FIG. 4C shows a schematic map of a Cre-LoxP donor vector according to an embodiment of the invention made by inserting the HBoV1 NS1 synthetic DNA (SEQ ID NO: 4) into the Cre-LoxP donor vector.

FIGS. 5A-5D are schematic representation of Replication (Rep) gene expression constructs according to the embodiments of invention. FIG. 5A shows a schematic linear map of a synthetic DNA encoding Sf-codon-optimized HBoV1 NS1 gene under the OpMNPV immediate-early2 (OpIE2) promoter followed by the SV40 polyadenylation signal (SV40 PAS). FIG. 5B shows a schematic linear map of a synthetic DNA encoding Sf-codon-optimized HBoV1 NS1 gene under the AcMNPV immediate-early1 (pIE1) promoter followed by the SV40 polyadenylation signal (SV40 PAS). FIG. 5C shows a schematic map of a Tn7 transfer vector according to an embodiment of the invention made by inserting the HBoV1 NS1 synthetic DNA (SEQ ID NO: 4) into the pFastBac1 vector (Invitrogen). FIG. 5D shows a schematic map of a Cre-LoxP donor vector according to an embodiment of the invention made by inserting the HBoV1 NS1 synthetic DNA (SEQ ID NO: 4) under the AcMNPV immediate-early1 (pIE1) promoter into the Cre-LoxP donor vector created as described in U.S. Patent Application No. 63/069,073.

FIGS. 6A-6B shows the generation of recombinant baculovirus expression vector (BEV) encoding human FVIIIXTEN with HBoV1 ITRs. FIG. 6A is an agarose gel electrophoresis image of restriction enzyme mapping of recombinant BIVVBac bacmid clones encoding human FVIIIXTEN with HBoV1 ITRs (BIVVBac.mTTR.FVIIIXTEN.HBoV1.ITRs^{Tn7}). FIG. 6B is schematic representation of recombinant BEV encoding FVIIIXTEN expression cassette flanked by the HBoV1 ITRs (SEQ ID NO: 3) as indicated (AcBIVVBac.mTTR.FVIIIXTEN.HBoV1.ITRs^{Tn7}).

FIGS. 7A-7F are schematic representation of One BACs comprising of human FVIIIXTEN and Rep gene expression cassettes and confirmation studies of the same. FIGS. 7A-C are agaroge gel electrophoresis images of recombinant bacmid clones of BIVVBac(mTTR.FVIIIXTEN.HBoV1.ITRs)Polh.HBoV1.NS1^{LoxP} (FIG. 7A), BIVVBac(mTTR. FVIIIXTEN.HBoV1.ITRs)IE1.HBoV1.NS1^{LoxP} (FIG. 7B), and BIVVBac(mTTR.FVIIIXTEN. HBoV1.ITRs)HR5.IE1.HBoV1.NS1^{LoxP} (FIG. 7C) screened by the outside/inside PCR primers (SEQ ID NO: 5 and SEQ ID NO: 6). as indicated by the red arrows in FIGS. 7D-7F. FIG. 7D shows a schematic map of recombinant baculovirus expression vectors (BEV) encoding HBoV1 NS1 under the AcMNPV Polyhedrin (pPolh) promoter and FVIIIXTEN expression cassette flanked by the HBoV1 ITRs as indicated (AcBIVVBac(mTTR.FVIIIXTEN.HBoV1.ITRs)Polh.HBoV1. NS1^{LoxP}) FIG. 7E shows a schematic map of recombinant BEV encoding HBoV1 NS1 under the AcMNPV immediate-early1 (pIE1) promoter and FVIIIXTEN expression cassette flanked by the HBoV1 ITRs as indicated (AcBIVVBac(mTTR.FVIIIXTEN.HBoV1.ITRs)IE1.HBoV1. NS1^{LoxP}) FIG. 7F shows a schematic map of recombinant BEV encoding HBoV1 NS1 under the AcMNPV immediate-early1 promoter preceded by the AcMNPV transcriptional enhancer hr5 element (pHR5.IE1) and FVIIIXTEN expression cassette flanked by the HBoV1 ITRs as indicated (AcBIVVBac(mTTR.FVIIIXTEN.HBoV1.ITRs)HR5.IE1.HBoV1.NS1^{LoxP}).

FIGS. 8A-8C shows the production of human FVIIIXTEN ceDNA vector using One BAC approach according to one embodiment of the invention. FIG. 8A is a schematic diagram of One BAC approach of FVIIIXTEN ceDNA vector production in Sf9 cells using recombinant BEV encoding HBoV1 NS1 gene under the AcMNPV polyhedrin promoter and human FVIIIXTEN expression cassette flanked by the HBoV1 ITRs (AcBIVVBac(mTTR.FVIIIXTEN.HBoV1.ITRs)Polh.HBoV1.NS1^{LoxP}). FIG. 8B shows the schematic map of AcBIVVBac(mTTR.FVIIIXTEN.HBoV1.ITRs)Polh.HBoV1.NS1^{LoxP} BEV FIG. 8C is an agarose gel electrophoresis image of ceDNA vector isolated from Sf9 cells infected with titrated virus stock (P2) of (AcBIVVBac(mTTR.FVIIIXTEN.HBoV1.ITRs)Polh.HBoV1.NS1^{LoxP})BEV. The DNA bands corresponding to the size of FVIIIXTEN ceDNA (ceDNA), baculoviral DNA (vDNA) and Sf9 cell genomic DNA (gDNA) are indicated by arrows.

FIGS. 9A-9D show schematics of recombinant baculovirus expression vectors (BEVs) comprising sequence encoding HBoV1 NS1 and confirmation studies of the same. FIG. 9A is an agarose gel electrophoresis image of restriction enzyme mapping of recombinant bacmid clones of HBoV1.NS1 under the AcMNPV polyhedrin, immediate-early1 preceded by the AcMNPV transcriptional enhancer hr5 element or the OpMNPV immediate-early2 promoter (BIVVBac.Polh.HBoV1.NS1^{Tn7}, BIVVBac.HR5.IE1.HBoV1.NS1^{Tn7}, and BIVVBac.OpIE2. HBoV1.NS1^{Tn7},respectively). FIG. 9B shows a schematic map of AcBIVVBac.Polh.HBoV1. NS1^{Tn7}. FIG. 9C shows a schematic map of AcBIVVBac.HR5.IE1.HBoV1.NS1^{Tn7}. FIG. 9D shows a schematic map of AcBIVVBac.OpIE2.HBoV1.NS1^{Tn7}.

FIGS. 10A-10C shows the production of human FVIIIXTEN ceDNA vector using Two BAC approach according to one embodiment of the invention. FIG. 10A is a schematic diagram of Two BAC approach of FVIIIXTEN ceDNA vector production, where Sf9 cells are co-infected with a recombinant BEVs encoding FVIIIXTEN expression cassette flanked by the HBoV1 ITRs (AcBIVVBac.mTTR.FVIIIXTEN.HBoV1.ITRs^{Tn7}) and/or encoding HBoV1 NS1 gene under the AcMNPV polyhedrin promoter (AcBIVVBac.Polh.HBoV1.NS1^{Tn7}). FIG. 10B shows the schematic maps of AcBIVVBac.mTTR.FVIIIXTEN.HBoV1.ITRs^{Tn7} and AcBIVVBac.Polh.HBoV1.NS1^{Tn7} BEVs. FIG. 10C is an agarose gel electrophoresis images of ceDNA vector isolated from Sf9 cells co-infected at different MOIs of constant ratio or different ratios of constant MOI of AcBIVVBac.mTTR.FVIIIXTEN.HBoV1.ITRs^{Tn7} and AcBIVVBac.Polh.HBoV1.NS1^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 11A-11C shows the production of human FVIIIXTEN ceDNA vector using Two BAC approach according to one embodiment of the invention. FIG. 11A is a schematic diagram of Two BAC approach of FVIIIXTEN ceDNA vector production, where Sf9 cells are co-infected with a recombinant BEV encoding FVIIIXTEN expression cassette flanked by the HBoV1 ITRs (AcBIVVBac.mTTR.FVIIIXTEN.HBoV1.ITRs^{Tn7}) and/or encoding HBoV1 NS1 gene under the AcMNPV polyhedrin promoter (AcBIVVBac.Polh.HBoV1.NS1^{Tn7}) or immediate-early1 promoter preceded by the AcMNPV transcriptional enhancer hr5 element (AcBIVVBac.HR5.IE1.HBoV1.NS1^{Tn7}). FIG. 11B shows the schematic maps of AcBIVVBac.mTTR.FVIIIXTEN.HBoV1.ITRs^{Tn7} and AcBIVVBac.HR5.IE1.HBoV1. NS1^{Tn7} BEVs.

FIG. 11C is an agarose gel electrophoresis images of ceDNA vector isolated from Sf9 cells co-infected at different MOIs of AcBIVVBac.mTTR.FVIIIXTEN.HBoV1.ITRs^{Tn7} and AcBIVVBac.Polh.HBoV1.NS1^{Tn7} (left image) or AcBIVVBac.HR5.IE1.HBoV1.NS1^{Tn7} (right image) BEVs. The DNA bands corresponding to the size of FVIIIXTEN ceDNA vector (ceDNA), baculoviral DNA (vDNA) and Sf9 cell genomic DNA (gDNA) are indicated by arrows.

FIGS. 12A-12C shows the materials used in generating stable cell line encoding FVIIIXTEN expression cassette flanked by the HBoV1 ITRs. FIG. 12A shows a schematic map of a plasmid encoding neomycin resistance marker under the AcMNPV immediate-early1 (IE1) promoter preceded by the AcMNPV transcriptional enhancer hr5 element and followed by the AcMNPV p10 polyadenylation signal (P10 PAS). FIG. 12B shows a schematic map of a plasmid encoding enhanced green fluorescent protein (eGFP) marker under the AcMNPV immediate-early1 (IE1) promoter preceded by the AcMNPV transcriptional enhancer hr5 element and followed by the AcMNPV p10 polyadenylation signal (P10 PAS). FIG. 12C shows a schematic map of the FVIIIXTEN expression cassette flanked by the HBoV1 ITRs (SEQ ID NO: 3), which was stably integrated into the Sf9 cell genome to generate stable cell line.

FIG. 13A-13E shows the workflow of FVIIIXTEN ceDNA vector production and purification using Two BAC approach according to one embodiment of the invention. FIG. 13A shows a schematic of Sf9 cell expansion and duration (Day 0-2), where the cells are sequentially scaled up from small scale (0.5 L) to large scale culture (1.5 L) flasks to achieve the cell density of 2.5 to 3.0×10⁶/mL in serum-free ESF921 medium. FIG. 13B shows a schematic of Sf9 large culture (1.5 L) flask infection and duration of incubation (Day 2-6), where the cells are co-infected with a recombinant BEV encoding a recombinant BEV encoding FVIIIXTEN expression cassette flanked by the HBoV1 ITRs (AcBIVVBac.mTTR.FVIIIXTEN.HBoV1.ITRs^{Tn7}) and/or encoding HBoV1 NS1 gene under the AcMNPV polyhedrin promoter (AcBIVVBac.Polh.HBoV1.NS1^{Tn7}) at an MOI of 0.1 and 0.01 plaque-forming units (pfu)/cell, respectively. FIG. 13C shows an image of Plasmid Giga Prep Purification kit and agarose gel electrophoresis with duration of processing (Day 6-7), where the cell density and viability of infected cells are measured daily, and the cells were pelleted by low-speed centrifugation once the cell viability reached at 70-80%. The FVIIIXTEN ceDNA vector was purified from infected cell pellets by the PureLink™ HiPure Expi Plasmid Gigaprep Kit (Invitrogen) and an aliquot was ran on agarose gel electrophoresis to determine the productivity of FVIIIXTEN ceDNA (ceDNA), baculoviral DNA (vDNA) and/or Sf9 cell genomic DNA (gDNA). FIG. 13D shows an image of Bio-Rad Model 491 Prep Cell and agarose gel electrophoresis with duration of processing (Day 7-12), where the Giga-prep purified DNAs were loaded onto Preparative agarose gel in Prep Cell for separating the FVIIIXTEN ceDNA (˜8.5 kb fragment) from the high molecular weight DNAs. Elution fractions collected at 70-80 min intervals from the Preparative Agarose Gel Electrophoresis were analyzed on 0.8 to 1.2% agarose gel to determine the purity of FVIIIXTEN ceDNA. FIG. 13E shows an image of agarose gel electrophoresis, where the fractions collected from the Prep Cell were combined and precipitated with 1/10^{th} vol of 3M NaOAc (pH 5.5) and 3 vol of 100% ethanol to obtain purified FVIIIXTEN ceDNA. The gel image shows the purity of FVIIIXTEN ceDNA in comparison with the starting material with arrows indicating DNA bands corresponding to the size of FVIIIXTEN ceDNA vector (ceDNA), baculoviral DNA (vDNA) and Sf9 cell genomic DNA (gDNA).

FIG. 14A-14B shows the graphical representation of plasma FVIII activity levels measured by the Chromogenix Coatest® SP Factor VIII chromogenic assays. FIG. 14A shows the graphical plot of plasma FVIII activity levels measured in blood samples collected at different intervals from hFVIIIR593C^{+/+}/HemA mice systemically injected via hydrodynamic tail-vein injection with 1600 or 400 μg/kg of single-stranded FVIIIXTEN HBoV1 ITRs DNA (ssDNA). FIG. 14B shows the graphical plot of plasma FVIII activity levels measured in blood samples collected at different intervals from hFVIIIR593C^{+/+}/HemA mice systemically injected via hydrodynamic tail-vein injection with 80, 40, or 12 μg/kg of FVIIIXTEN HBoV1 ITRs ceDNA (ceDNA). Error bars represents standard deviation.

FIGS. 15A-15C shows the red fluorescence (upper panel) or brightfield (lower panel) microscopic images of Sf9 cells co-transfected with AcBIVVBac.Polh.HBoV1.NS1^{Tn7} bacmid DNA and VP80 sgRNAs according to one embodiment of the invention. FIG. 15A shows the microscopic images of cells co-transfected with AcBIVVBac.Polh.HBoV1.NS1^{Tn7} bacmid DNA and Cas9 alone. FIG. 15B shows the microscopic images of cells co-transfected with AcBIVVBac.Polh.HBoV1.NS1^{Tn7} bacmid DNA and sgRNA.VP80.T1. FIG. 15C shows the microscopic images of cells co-transfected with AcBIVVBac.Polh.HBoV1.NS1^{Tn7} bacmid DNA and sgRNA.VP80.T2.

FIGS. 16A-16C show the generation of VP80KO BEVs. FIG. 16A and FIG. 16B illustrate the TIDE analyses of AcBIVVBac.Polh.HBoV1.NS1ΔVP80^{Tn7} and AcBIVVBac.OpIE2.HBoV1.NS1ΔVP80^{Tn7} clonal BEVs to determine the indels induced by CRISPR/Cas9. FIG. 16C is an agarose gel electrophoresis image of FVIIIXTEN ceDNA vector isolated from Sf9 cells co-infected at different MOIs of AcBIVVBac.mTTR.FVIIIXTEN.HBoV1.ITRs^{Tn7} and AcBIVVBac.Polh.HBoV1.NS1ΔVP80^{Tn7} or AcBIVVBac.OpIE2.HBoV1.NS1ΔVP80^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 17A-17C shows the production of human FVIIIXTEN HBoV1 ceDNA using the TwoBAC approach. FIG. 17A is a schematic diagram of the TwoBAC approach of FVIIIXTEN ceDNA vector production. FIG. 17B shows the schematic maps of AcBIVVBac.mTTR.FVIIIXTEN.HBoV1.ITRs^{Tn7} and AcBIVVBac.Polh.HBoV1.NS1^{Tn7} BEVs. FIG. 17C is an agarose gel electrophoresis image of FVIIIXTEN ceDNA vector isolated from Sf9 cells co-infected with AcBIVVBac.mTTR.FVIIIXTEN.HBoV1.ITRs^{Tn7} and AcBIVVBac.Polh.HBoV1.NS1^{Tn7} BEVs at 1.0, 2.0, 3.0, 4.0, or 5.0 MOI. The DNA bands corresponding to the size of FVIIIXTEN ceDNA vector (ceDNA), baculoviral DNA (vDNA) and Sf9 cell genomic DNA (gDNA) are indicated by arrows.

FIGS. 18A-18D shows the production of human FVIIIXTEN HBoV1 ceDNA vector using the OneBAC approach. FIG. 18A is a schematic diagram of OneBAC approach of FVIIIXTEN ceDNA vector production in Sf9 cells. FIG. 18B shows the schematic map of AcBIVVBac(mTTR.FVIIIXTEN.HBoV1.ITRs)Polh.HBoV1.NS1^{LoxP} BEV. FIG. 18C is an agarose gel electrophoresis image of ceDNA isolated from clonal HBoV1 OneBAC BEVs amplified to P2 in Sf9 cells. FIG. 18D is an agarose gel electrophoresis image of ceDNA isolated from Sf9 cells infected with HBoV1 OneBAC BEV at 0.1, 0.2, 0.3, 0.4, or 0.5 MOIs. The DNA bands corresponding to the size of FVIIIXTEN ceDNA (ceDNA), baculoviral DNA (vDNA) and Sf9 cell genomic DNA (gDNA) are indicated by arrows.

FIG. 19A-19C shows the generation and testing of HBoV1 ssDNA and ceDNA in vivo. FIG. 19A is an agarose gel electrophoresis image of single-stranded DNA (ssDNA) FVIIIXTEN HBoV1. FIG. 19B is an agarose gel electrophoresis image of FVIIIXTEN HBoV1 ceDNA. FIG. 19C shows the FVIII expression levels normalized to percent of normal for ssFVIIIXTEN and ceFVIIIXTEN. Error bars represent standard deviation.

FIG. 20A-20B shows the testing of monomeric and multimeric forms of FVIIIXTEN HBoV1 ceDNA. FIG. 20A is an agarose gel electrophoresis image of monomeric and multimeric forms of FVIIIXTEN HBoV1 ceDNA. FIG. 20B shows the FVIII expression levels normalized to percent of normal in mice injected with monomeric and multimeric forms of FVIIIXTEN HBoV1 ceDNA. Error bars represent standard deviation.

FIG. 21A-21C shows the testing of the liver-specific mTTR and human A1AT promoter driving expression of FVIIIXTEN in HBoV1 ITR constructs. FIG. 21A is a schematic diagram of FVIIIXTEN expression cassettes with liver-specific mTTR or A1AT promoter flanked by HBoV1 WT ITRs. FIG. 21B is an agarose gel electrophoresis image of single-stranded DNA (ssDNA) FVIIIXTEN HBoV1 generated by restriction enzyme digestion as described. FIG. 21C shows the FVIII expression levels normalized to percent of normal in mice injected with the mTTR or A1AT promoter constructs depicted in FIG. 21A. Error bars represent standard deviation.

### DETAILED DESCRIPTION

Disclosed herein are nucleic acid molecules and uses thereof comprising a modified first inverted terminal repeat (ITR) and/or a modified second ITR flanking a genetic cassette comprising a heterologous polynucleotide sequence. In some embodiments, the first and/or second ITR is derived from human bocavirus 1 (HBoV1).

Exemplary constructs of the disclosure are illustrated in the accompanying figures and sequence listing. In order to provide a clear understanding of the specification and claims, the following definitions are provided below.

### Definitions

It is to be noted that the term “a” or “an” entity refers to one or more of that entity: for example, “a nucleotide sequence” is understood to represent one or more nucleotide sequences. Similarly, “a therapeutic protein” and “a miRNA” is understood to represent one or more therapeutic protein and one or more miRNA, respectively. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Also as used herein, “and/or” refers to and encompasses any and all possible combinations of one or more of the associated listed items, as well as the lack of combinations when interpreted in the alternative (“or”).

“Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

In one embodiment, the initial sequence of the ITR and/or the reverse complement comprise about 2-600 nucleotides, about 2-550 nucleotides, about 2-500 nucleotides, about 2-450 nucleotides, about 2-400 nucleotides, about 2-350 nucleotides, about 2-300 nucleotides, or about 2-250 nucleotides. In some embodiments, the initial sequence and/or the reverse complement comprise about 5-600 nucleotides, about 10-600 nucleotides, about 15-600 nucleotides, about 20-600 nucleotides, about 25-600 nucleotides, about 30-600 nucleotides, about 35-600 nucleotides, about 40-600 nucleotides, about 45-600 nucleotides, about 50-600 nucleotides, about 60-600 nucleotides, about 70-600 nucleotides, about 80-600 nucleotides, about 90-600 nucleotides, about 100-600 nucleotides, about 150-600 nucleotides, about 200-600 nucleotides, about 300-600 nucleotides, about 350-600 nucleotides, about 400-600 nucleotides, about 450-600 nucleotides, about 500-600 nucleotides, or about 550-600 nucleotides. In some embodiments, the initial sequence and/or the reverse complement comprise about 5-550 nucleotides, about 5 to 500 nucleotides, about 5-450 nucleotides, about 5 to 400 nucleotides, about 5-350 nucleotides, about 5 to 300 nucleotides, or about 5-250 nucleotides. In some embodiments, the initial sequence and/or the reverse complement comprise about 10-550 nucleotides, about 15-500 nucleotides, about 20-450 nucleotides, about 25-400 nucleotides, about 30-350 nucleotides, about 35-300 nucleotides, or about 40-250 nucleotides. In certain embodiments, the initial sequence and/or the reverse complement comprise about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, or about 600 nucleotides. In particular embodiments, the initial sequence and/or the reverse complement comprise about 400 nucleotides.

In other embodiments, the initial sequence of the ITR and/or the reverse complement comprise about 2-200 nucleotides, about 5-200 nucleotides, about 10-200 nucleotides, about 20-200 nucleotides, about 30-200 nucleotides, about 40-200 nucleotides, about 50-200 nucleotides, about 60-200 nucleotides, about 70-200 nucleotides, about 80-200 nucleotides, about 90-200 nucleotides, about 100-200 nucleotides, about 125-200 nucleotides, about 150-200 nucleotides, or about 175-200 nucleotides. In other embodiments, the initial sequence and/or the reverse complement comprise about 2-150 nucleotides, about 5-150 nucleotides, about 10-150 nucleotides, about 20-150 nucleotides, about 30-150 nucleotides, about 40-150 nucleotides, about 50-150 nucleotides, about 75-150 nucleotides, about 100-150 nucleotides, or about 125-150 nucleotides. In other embodiments, the initial sequence and/or the reverse complement comprise about 2-100 nucleotides, about 5-100 nucleotides, about 10-100 nucleotides, about 20-100 nucleotides, about 30-100 nucleotides, about 40-100 nucleotides, about 50-100 nucleotides, or about 75-100 nucleotides. In other embodiments, the initial sequence and/or the reverse complement comprise about 2-50 nucleotides, about 10-50 nucleotides, about 20-50 nucleotides, about 30-50 nucleotides, about 40-50 nucleotides, about 3-30 nucleotides, about 4-20 nucleotides, or about 5-10 nucleotides. In another embodiment, the initial sequence and/or the reverse complement consist of two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In other embodiments, an intervening nucleotide between the initial sequence and the reverse complement is (e.g., consists of) 0 nucleotide, 1 nucleotide, two nucleotides, three nucleotides, four nucleotides, five nucleotides, six nucleotides, seven nucleotides, eight nucleotides, nine nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop. In some embodiments, the ITR retains the Rep Binding Element (RBE) of the wild type ITR from which it is derived. Preservation of the RBE may be important for stability of the ITR and manufacturing purposes.

The term “parvovirus” as used herein encompasses the family Parvoviridae, including but not limited to autonomously replicating parvoviruses and Dependoviruses. The autonomous parvoviruses include, for example, members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus. Exemplary autonomous parvoviruses include, but are not limited to, human bocavirus 1 (HBoV1), porcine parvovirus, mice minute virus, canine parvovirus, mink entertitus virus, bovine parvovirus, chicken parvovirus, feline panleukopenia virus, feline parvovirus, goose parvovirus (GPV), H1 parvovirus, muscovy duck parvovirus, snake parvovirus, and B19 virus. Other autonomous parvoviruses are known to those skilled in the art. See, e.g., Fields et al. Virology, Vol. 2, Ch. 69 (4th ed., Lippincott-Raven Publishers).

The term “non-AAV as used herein encompasses nucleic acids, proteins, and viruses from the family Parvoviridae excluding any adeno-associated viruses (AAV) of the Parvoviridae family. “Non-AAV” includes but is not limited to autonomously replicating members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers).

The term “derived from,” as used herein, refers to a component that is isolated from or made using a specified molecule or organism, or information (e.g., amino acid or nucleic acid sequence) from the specified molecule or organism. For example, a nucleic acid sequence (e.g., ITR) that is derived from a second nucleic acid sequence (e.g., ITR) can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide.

A “capsid-free” or “capsid-less” vector or nucleic acid molecule refers to a vector construct free from a capsid.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse B-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “genetic cassette” means a DNA sequence capable of directing expression of a particular polynucleotide sequence in an appropriate host cell, comprising a promoter operably linked to a polynucleotide sequence of interest. A genetic cassette may encompass nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence. In some embodiments, the genetic cassette comprises a polynucleotide which encodes a gene product. In some embodiments, the genetic cassette comprises a polynucleotide which encodes a miRNA. In some embodiments, the genetic cassette comprises a heterologous polynucleotide sequence.

A polynucleotide which encodes a product, e.g., a miRNA or a gene product (e.g., a polypeptide such as a therapeutic protein), can include a promoter and/or other expression (e.g., transcription or translation) control sequences operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control sequences, besides a promoter, for example enhancers, operators, repressors, and transcription termination signals, can also be operably associated with a coding region to direct gene product expression.

“Expression control sequences” refer to regulatory nucleotide sequences, such as promoters, enhancers, terminators, and the like, that provide for the expression of a coding sequence in a host cell. Expression control sequences generally encompass any regulatory nucleotide sequence which facilitates the efficient transcription and translation of the coding nucleic acid to which it is operably linked. Non-limiting examples of expression control sequences include include promoters, enhancers, translation leader sequences, introns, polyadenylation recognition sequences, RNA processing sites, effector binding sites, or stem-loop structures. A variety of expression control sequences are known to those skilled in the art. These include, without limitation, expression control sequences which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other expression control sequences include those derived from vertebrate genes such as actin, heat shock protein, bovine growth hormone and rabbit B-globin, as well as other sequences capable of controlling gene expression in eukaryotic cells. Additional suitable expression control sequences include tissue-specific promoters and enhancers as well as lymphokine-inducible promoters (e.g., promoters inducible by interferons or interleukins). Other expression control sequences include intronic sequences, post-transcriptional regulatory elements, and polyadenylation signals. Additional exemplary expression control sequences are discussed elsewhere in the present disclosure.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity. Additional exemplary promoters are discussed elsewhere in the present disclosure.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In some embodiments, the nucleic acid molecule comprises a tissue specific promoter. In certain embodiments, the tissue specific promoter drives expression of the therapeutic protein in the liver, in hepatocytes, and/or endothelial cells. In one particular embodiment, the promoter comprises a TTP promoter. In one particular embodiment, the promoter comprises a mTTR promoter. In one particular embodiment, the promoter comprises a A1AT promoter.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construct, which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, Wis.), the GCG suite of programs (Wisconsin Package Version 9.0, Genetics Computer Group (GCG), Madison, Wis.), BLASTP, BLASTN, BLASTX (Altschul et al., J. Mol. Biol. 215:403 (1990)), and DNASTAR (DNASTAR, Inc. 1228 S. Park St. Madison, Wis. 53715 USA). Within the context of this application, it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between a query sequence (e.g., a nucleic acid sequence) and a reference sequence, only nucleotides in the query sequence which match to nucleotides in the reference sequence are used to calculate percent identity. Thus, in determining percent identity between a query sequence or a designated portion thereof (e.g., nucleotides 1-522) and a reference sequence, percent identity will be calculated by dividing the number of matched nucleotides by the total number of nucleotides in the complete query sequence.

As used herein, nucleotides corresponding to nucleotides in a particular sequence of the disclosure are identified by alignment of the sequence of the disclosure to maximize the identity to a reference sequence. The number used to identify an equivalent amino acid in a reference sequence is based on the number used to identify the corresponding amino acid in the sequence of the disclosure.

“Treat,” “treatment,” “treating,” as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition.

“Administering,” as used herein, means to give a pharmaceutically acceptable nucleic acid molecule, polypeptide expressed therefrom, or vector comprising the nucleic acid molecule of the disclosure to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The nucleic acid molecules, polypeptides, and vectors can be administered as part of a pharmaceutical composition comprising at least one excipient.

“Lipid nanoparticle”, as used herein, refers to a particle having at least one dimension on the nanometer scale (e.g., 1 nm to 1,000 nm) comprising one or more cationic lipids. In some embodiments, the lipid nanoparticle is included in a formulation that can be used to deliver an active or therapeutic agent, such as a nucleic acid (e.g., mRNA), to an associated target site (e.g., cell, tissue, organ, tumor, etc.). In some embodiments, the lipid nanoparticle disclosed herein comprises a nucleic acid. Such lipid nanoparticles typically comprise one or more excipients selected from neutral lipids, charged lipids, steroids and polymer-conjugated lipids. In some embodiments, an active or therapeutic agent, such as a nucleic acid, may be encapsulated in the lipid portion of the lipid nanoparticle, or in the aqueous space encapsulated by some or all of the lipid portion of the lipid nanoparticle, thereby protecting it from enzymatic degradation, or other undesirable effects triggered by the mechanisms of the host organism or cell, such as an adverse immune response.

The term “pharmaceutically acceptable” as used herein refer to molecular entities and compositions that are physiologically tolerable and do not typically produce toxicity or an allergic or similar untoward reaction, such as gastric upset, dizziness and the like, when administered to a human. Optionally, as used herein, the term “pharmaceutically acceptable” means approved by a regulatory agency of the federal or a state government or listed in the U.S. Pharmacopeia or other generally recognized pharmacopeia for use in animals, and more particularly in humans.

As used herein, the phrase “subject in need thereof” includes subjects, such as mammalian subjects, that would benefit from administration of a nucleic acid molecule, polypeptide, or vector of the disclosure. In some embodiments, the subject is a human subject. In some embodiments, the subjects are individuals with hemophilia. The subject can be an adult or a minor (e.g., under 12 years old).

As used herein, the term “therapeutic protein” refers to any polypeptide known in the art that can be administered to a subject. In some embodiments, the therapeutic protein comprises a protein selected from a clotting factor, a growth factor, an antibody, a functional fragment thereof, or a combination thereof. As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. “Clotting factor” as used herein includes an activated clotting factor, its zymogen, or an activatable clotting factor. An “activatable clotting factor” is a clotting factor in an inactive form (e.g., in its zymogen form) that is capable of being converted to an active form. The term “clotting factor” includes but is not limited to factor I (FI), factor II (FII), factor III (FIII), factor IV (FIV), factor V (FV), factor VI (FVI), factor VII (FVII), factor VIII (FVIII), factor IX (FIX), factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), zymogens thereof, activated forms thereof, or any combination thereof.

“Clotting activity,” as used herein, means the ability to participate in a cascade of biochemical reactions that culminates in the formation of a fibrin clot and/or reduces the severity, duration or frequency of hemorrhage or bleeding episode.

A “growth factor,” as used herein, includes any growth factor known in the art including cytokines and hormones.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

A “reference nucleotide sequence,” when used herein as a comparison to a nucleotide sequence of the disclosure, is a polynucleotide sequence essentially identical to the nucleotide sequence of the disclosure except that the portions corresponding to FVIII sequence are not optimized. In some embodiments, the reference nucleotide sequence for a nucleic acid molecule disclosed herein is SEQ ID NO: 32.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, removal of CpG motifs, and two or more combinations thereof.

### Nucleic Acid Molecules

Certain aspects of the present disclosure aim to overcome deficiencies of AAV vectors for gene therapy. In particular, certain aspects of the present disclosure are directed to a nucleic acid molecule, comprising a first ITR, a second ITR, and a genetic cassette. In some embodiments, the genetic cassette encodes a therapeutic protein and/or a miRNA. In some embodiments, the first ITR and second ITR flank a genetic cassette comprising a heterologous polynucleotide sequence. In some embodiments, the nucleic acid molecule does not comprise a gene encoding a capsid protein, a replication protein, and/or an assembly protein. In some embodiments, the genetic cassette encodes a therapeutic protein. In some embodiments, the therapeutic protein comprises a clotting factor. In some embodiments, the genetic cassette encodes a miRNA. In certain embodiments, the genetic cassette is positioned between the first ITR and the second ITR. In some embodiments, the nucleic acid molecule further comprises one or more noncoding region. In certain embodiments, the one or more non-coding region comprises a promoter sequence, an intron, a post-transcriptional regulatory element, a 3′UTR poly(A) sequence, or any combination thereof.

In one embodiment, the genetic cassette is a single stranded nucleic acid. In another embodiment, the genetic cassette is a double stranded nucleic acid. In another embodiment, the genetic cassette is a closed-end double stranded nucleic acid (ceDNA).

In some embodiments, the nucleic acid molecule comprises: (a) a first ITR that is an ITR derived from a non-AAV family member of Parvoviridae (e.g., a HBoV1 ITR); (b) a tissue specific promoter sequence, e.g., TTP or TTR promoter; (c) an intron, e.g., a synthetic intron; (d) a nucleotide encoding a miRNA or a therapeutic protein, e.g., a clotting factor; (e) a post-transcriptional regulatory element, e.g., WPRE; (f) a 3′ UTR poly(A) tail sequence, e.g., bGHpA; (g) a second ITR that is an ITR derived from a non-AAV family member of Parvoviridae (e.g., a HBoV1 ITR). In some embodiments, the nucleic acid molecule comprises: (a) a first ITR that is an ITR derived from a non-AAV family member of Parvoviridae (e.g., a HBoV1 ITR); (b) a tissue specific promoter sequence, e.g., mTTR promoter; (c) an intron, e.g., a synthetic intron; (d) a nucleotide encoding a miRNA or a therapeutic protein, e.g., a clotting factor; (e) a post-transcriptional regulatory element, e.g., WPRE; (f) a 3′UTR poly(A) tail sequence, e.g., bGHpA; (g) a second ITR that is an ITR derived from a non-AAV family member of Parvoviridae (e.g., a HBoV1 ITR). In some embodiments, the tissue specific promoter is the human alpha-1-antitrypsin (A1AT) promoter. In some embodiments, the tissue specific promoter comprises the nucleotide sequence of SEQ ID NO: 36.

In some embodiments, disclosed herein are isolated nucleic acid molecules comprising a genetic cassette comprising a nucleotide sequence at least about 75% identical to SEQ ID NO: 9. In some embodiments, disclosed herein is a nucleic acid molecule comprising a nucleotide sequence having at least 50%, 55%, 60%, 65%, 70%, 75%, 80%, 85%, 90%, 95%, or 100% sequence identity to SEQ ID NO: 9.

In some embodiments, disclosed herein are isolated nucleic acid molecules comprising a genetic cassette comprising a nucleotide sequence at least about 75% identical to SEQ ID NO: 33. In some embodiments, disclosed herein is a nucleic acid molecule comprising a nucleotide sequence having at least 50%, 55%, 60%, 65%, 70%, 75%, 80%, 85%, 90%, 95%, or 100% sequence identity to SEQ ID NO: 33.

In some embodiments, disclosed herein are isolated nucleic acid molecules comprising a genetic cassette comprising a nucleotide sequence at least about 75% identical to SEQ ID NO: 14. In some embodiments, disclosed herein is a nucleic acid molecule comprising a nucleotide sequence having at least 50%, 55%, 60%, 65%, 70%, 75%, 80%, 85%, 90%, 95%, or 100% sequence identity to SEQ ID NO: 14.

In another aspect, disclosed herein is an isolated nucleic acid molecule comprising a genetic cassette expressing a factor VIII (FVIII) polypeptide, wherein the genetic cassette comprises a nucleotide sequence at least 85% identical to SEQ ID NO: 35. In some embodiments, the genetic cassette comprises a nucleotide sequence that is at least 90% identical to SEQ ID NO: 35. In some embodiments, the genetic cassette comprises a nucleotide sequence that is at least 95%, at least 96%, at least 97%, at least 98%, at least 99%, or 100% identical to SEQ ID NO: 35. In some embodiments, the nucleotide sequence is at least 50% identical to SEQ ID NO: 35.

Also disclosed herein is an isolated nucleic acid molecule comprising a genetic cassette expressing a factor VIII (FVIII) polypeptide, wherein the genetic cassette comprises the nucleotide sequence of SEQ ID NO: 35.

In certain embodiments, the nucleic acid molecule disclosed herein comprises ITR sequences from human bocavirus 1 (HBoV1). In certain embodiments, the nucleic acid molecule disclosed herein comprises a first ITR that is at least about 75% identical to SEQ ID NO: 1 or SEQ ID NO: 2.

A. Inverted Terminal Repeats (ITRs)

Certain aspects of the present disclosure are directed to a nucleic acid molecule comprising a first ITR, e.g., a 5′ ITR, and second ITR, e.g., a 3′ ITR. Typically, ITRs are involved in parvovirus (e.g., AAV) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. It is hypothesized that the minimal defining elements indispensable for ITR function are a Rep-binding site and a terminal resolution site plus a variable palindromic sequence allowing for hairpin formation. Palindromic nucleotide regions normally function together in cis as origins of DNA replication and as packaging signals for the virus. Complimentary sequences in the ITRs fold into a hairpin structure during DNA replication. In some embodiments, the ITRs fold into a hairpin T-shaped structure. In other embodiments, the ITRs fold into non-T-shaped hairpin structures, e.g., into a U-shaped hairpin structure. Data suggests that the T-shaped hairpin structures of AAV ITRs may inhibit the expression of a transgene flanked by the ITRs. See, e.g., Zhou et al. (2017) Scientific Reports 7:5432. By utilizing an ITR that does not form T-shaped hairpin structures, this form of inhibition may be avoided. Therefore, in certain aspects, a polynucleotide comprising a non-AAV ITR has an improved transgene expression compared to a polynucleotide comprising an AAV ITR that forms a T-shaped hairpin.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop.

In some embodiments, the ITR comprises a naturally occurring ITR, e.g. the ITR comprises all or a portion of an ITR derived from a member of the family Parvoviridae. In some embodiments, the ITR comprises a synthetic sequence. In one embodiment, the first ITR or the second ITR comprises a synthetic sequence. In another embodiment, each of the first ITR and the second ITR comprises a synthetic sequence. In some embodiments, the first ITR or the second ITR comprises a naturally occurring sequence. In another embodiment, each of the first ITR and the second ITR comprises a naturally occurring sequence.

In some embodiments, the ITR comprises or consists of a portion of a naturally occurring ITR, e.g., a truncated ITR. In some embodiments, the ITR comprises or consists of a fragment of a naturally occurring ITR, wherein the fragment comprises at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 125 nucleotides, at least about 150 nucleotides, at least about 175 nucleotides, at least about 200 nucleotides, at least about 225 nucleotides, at least about 250 nucleotides, at least about 275 nucleotides, at least about 300 nucleotides, at least about 325 nucleotides, at least about 350 nucleotides, at least about 375 nucleotides, at least about 400 nucleotides, at least about 425 nucleotides, at least about 450 nucleotides, at least about 475 nucleotides, at least about 500 nucleotides, at least about 525 nucleotides, at least about 550 nucleotides, at least about 575 nucleotides, or at least about 600 nucleotides; wherein the ITR retains a functional property of the naturally occurring ITR. In certain embodiments, the ITR comprises or consists of a fragment of a naturally occurring ITR, wherein the fragment comprises at least about 129 nucleotides; wherein the ITR retains a functional property of the naturally occurring ITR. In certain embodiments, the ITR comprises or consists of a fragment of a naturally occurring ITR, wherein the fragment comprises at least about 102 nucleotides; wherein the ITR retains a functional property of the naturally occurring ITR. In some embodiments, the ITR retains the Rep Binding Element (RBE) of the wild type ITR from which it is derived. In some embodiments, the ITR retains at least one of the RBEs of the wild type ITR from which it is derived. In some embodiments, the ITR retains at least one of the RBEs or a functional portion thereof of the wild type ITR from which it is derived. Preservation of the RBE may be important for stability of the ITR and manufacturing purposes.

In some embodiments, the ITR comprises or consists of a portion of a naturally occurring ITR, wherein the fragment comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the length of the naturally occurring ITR; wherein the fragment retains a functional property of the naturally occurring ITR. In some embodiments, the first ITR and/or the second ITR is derived from a wild type HBoV1 ITR. In some embodiments, the first ITR and/or the second ITR is derived from a wild type B19 ITR. In some embodiments, the first ITR and/or the second ITR is derived from a wild type GPV ITR.

In certain embodiments, the ITR comprises or consists of a sequence that has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to a homologous portion of a naturally occurring ITR, when properly aligned; wherein the ITR retains a functional property of the naturally occurring ITR. In other embodiments, the ITR comprises or consists of a sequence that has a sequence identity of at least 90% to a homologous portion of a naturally occurring ITR, when properly aligned; wherein the ITR retains a functional property of the naturally occurring ITR. In some embodiments, the ITR comprises or consists of a sequence that has a sequence identity of at least 80% to a homologous portion of a naturally occurring ITR, when properly aligned; wherein the ITR retains a functional property of the naturally occurring ITR. In some embodiments, the ITR comprises or consists of a sequence that has a sequence identity of at least 70% to a homologous portion of a naturally occurring ITR, when properly aligned; wherein the ITR retains a functional property of the naturally occurring ITR. In some embodiments, the ITR comprises or consists of a sequence that has a sequence identity of at least 60% to a homologous portion of a naturally occurring ITR, when properly aligned; wherein the ITR retains a functional property of the naturally occurring ITR. In some embodiments, the ITR comprises or consists of a sequence that has a sequence identity of at least 50% to a homologous portion of a naturally occurring ITR, when properly aligned; wherein the ITR retains a functional property of the naturally occurring ITR.

In some embodiments, the ITR comprises an ITR from an AAV genome. In some embodiments, the ITR is an ITR of an AAV genome selected from AAV1, AAV2, AAV3, AAV4, AAV5, AAV6, AAV7, AAV8, AAV9, AAV10, and AAV11, and any combination thereof. In some embodiments, the ITR is an ITR of any AAV genome known to those of skill in the art, including a natural isolate, e.g., a natural human isolate. In a particular embodiment, the ITR is an ITR of the AAV2 genome. In another embodiment, the ITR is a synthetic sequence genetically engineered to include at its 5′ and 3′ ends ITRs derived from one or more of AAV genomes.

In some embodiments, the ITR is not derived from an AAV genome (i.e. the ITR is derived from a virus that is not AAV). In some embodiments, the ITR is an ITR of a non-AAV. In some embodiments, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain embodiments, the ITR is derived from human bocavirus 1 (HBoV1). In another embodiment, the ITR is derived from erythrovirus parvovirus B19 (human virus). In another embodiment, the ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY. In some embodiments, the ITR is derived from a porcine parvovirus, e.g., porcine parvovirus U44978. In some embodiments, the ITR is derived from a mice minute virus, e.g., mice minute virus U34256. In some embodiments, the ITR is derived from a canine parvovirus, e.g., canine parvovirus M19296. In some embodiments, the ITR is derived from a mink enteritis virus, e.g., mink enteritis virus D00765. In some embodiments, the ITR is derived from a Dependoparvovirus. In one embodiment, the Dependoparvovirus is a Dependovirus Goose parvovirus (GPV) strain. In a specific embodiment, the GPV strain is attenuated, e.g., GPV strain 82-0321V. In another specific embodiment, the GPV strain is pathogenic, e.g., GPV strain B.

The first ITR and the second ITR of the nucleic acid molecule can be derived from the same genome, e.g., from the genome of the same virus, or from different genomes, e.g., from the genomes of two or more different virus genomes. In certain embodiments, the first ITR and the second ITR are derived from the same AAV genome. In a specific embodiment, the two ITRs present in the nucleic acid molecule of the invention are the same and can in particular be AAV2 ITRs. In other embodiments, the first ITR is derived from an AAV genome and the second ITR is not derived from an AAV genome (e.g., a non-AAV genome). In other embodiments, the first ITR is not derived from an AAV genome (e.g., a non-AAV genome) and the second ITR is derived from an AAV genome. In still other embodiments, both the first ITR and the second ITR are not derived from an AAV genome (e.g., a non-AAV genome). In one particular embodiment, the first ITR and the second ITR are identical.

In some embodiments, the first ITR is derived from a non-AAV genome and the second ITR is derived from a non-AAV genome, wherein the first ITR and the second ITR are derived from the same genome. Non-limiting examples of non-AAV viral genomes are from Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus. In some embodiments, the first ITR is derived from a non-AAV genome and the second ITR is derived from a non-AAV genome, wherein the first ITR and the second ITR are derived from different viral genomes.

In some embodiments, the first ITR is derived from an AAV genome, and the second ITR is derived from human bocavirus 1 (HBoV1). In other embodiments, the second ITR is derived from an AAV genome, and the first ITR is derived from human bocavirus 1 (HBoV1).

In some embodiments, the first ITR comprises or consists of all or a portion of an ITR derived from an AAV or non-AAV genome, and the second ITR comprises or consists of all or a portion of an ITR derived from an AAV or non-AAV genome. In some embodiments, a portion of an ITR derived from an AAV or non-AAV genome is a truncated version of a naturally occurring ITR derived from an AAV or non-AAV genome. In some embodiments, a portion of an ITR derived from an AAV or non-AAV genome comprises portions of a naturally occurring ITR derived from an AAV or non-AAV genome. For example, a portion of an ITR derived from an AAV or non-AAV genome comprises portions of a naturally occurring ITR derived from an AAV or non-AAV genome, wherein at least one RBE or a functional portion thereof is preserved.

In certain embodiments, the first ITR and/or the second ITR comprises or consists of all or a portion of an ITR derived from HBoV1. In certain embodiments, the first ITR and/or the second ITR comprises or consists of all or a portion of an ITR derived from HBoV1. In some embodiments, the second ITR is a reverse complement of the first ITR. In some embodiments, the first ITR is a reverse complement of the second ITR. In some embodiments, the first ITR and/or the second ITR derived from HBoV1 is capable of forming a hairpin structure. In certain embodiments, the hairpin structure does not comprise a T-shaped hairpin.

In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs: 1 or 2, wherein the first ITR and/or the second ITR retains a functional property of the HBoV1 ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 1 or 2, wherein the first ITR and/or the second ITR is capable of forming a hairpin structure. In certain embodiments, the hairpin structure does not comprise a T-shaped hairpin.

In some embodiments, the first ITR and/or the second ITR comprises or consists of the nucleotide sequence of SEQ ID NO: 1. In some embodiments, the first ITR and/or the second ITR comprises or consists of the nucleotide sequence of SEQ ID NO: 2. In some embodiments, the first ITR comprises or consists of the nucleotide sequence set forth in SEQ ID NO: 1. In some embodiments, the second ITR comprises or consists of the nucleotide sequence set forth in SEQ ID NO: 2. In some embodiments, the first ITR comprises or consists of the nucleotide sequence set forth in SEQ ID NO: 1 and the second ITR comprises or consists of the nucleotide sequence set forth in SEQ ID NO: 2.

It will be appreciated to those of skill in the art that any of the first ITR sequences described herein can be matched with any of the second ITR sequences described herein. In some embodiments, the first ITR sequence described herein is a 5′ ITR sequence. In some embodiments, the second ITR sequence described herein is a 3′ ITR sequence. In some embodiments, the second ITR sequence described herein is a 5′ ITR sequence. In some embodiments, the first ITR sequence described herein is a 3′ ITR sequence. Those of skill in the art will be able to determine the suitable orientation of the first and the second ITR described herein with respect to the architecture of a genetic cassette.

In another particular embodiment, the ITR is a synthetic sequence genetically engineered to include at its 5′ and 3′ ends ITRs not derived from an AAV genome. In another particular embodiment, the ITR is a synthetic sequence genetically engineered to include at its 5′ and 3′ ends ITRs derived from one or more of non-AAV genomes. The two ITRs present in the nucleic acid molecule of the invention can be the same or different non-AAV genomes. In particular, the ITRs can be derived from the same non-AAV genome. In a specific embodiment, the two ITRs present in the nucleic acid molecule of the invention are the same and can in particular be AAV2 ITRs.

In some embodiments, the ITR sequence comprises one or more palindromic sequence. A palindromic sequence of an ITR disclosed herein includes, but is not limited to, native palindromic sequences (i.e., sequences found in nature), synthetic sequences (i.e., sequences not found in nature), such as pseudo palindromic sequences, and combinations or modified forms thereof. A “pseudo palindromic sequence” is a palindromic DNA sequence, including an imperfect palindromic sequence, which shares less than 80% including less than 70%, 60%, 50%, 40%, 30%, 20%, 10%, or 5%, or no, nucleic acid sequence identity to sequences in native AAV or non-AAV palindromic sequence which form a secondary structure. The native palindromic sequences can be obtained or derived from any genome disclosed herein. The synthetic palindromic sequence can be based on any genome disclosed herein.

The palindromic sequence can be continuous or interrupted. In some embodiments, the palindromic sequence is interrupted, wherein the palindromic sequence comprises an insertion of a second sequence. In some embodiments, the second sequence comprises a promoter, an enhancer, an integration site for an integrase (e.g., sites for Cre or FIp recombinase), an open reading frame for a gene product, or a combination thereof.

In some embodiments, the ITRs form hairpin loop structures. In one embodiment, the first ITR forms a hairpin structure. In another embodiment, the second ITR forms a hairpin structure. Still in another embodiment, both the first ITR and the second ITR form hairpin structures. In some embodiments, the first ITR and/or the second ITR does not form a T-shaped hairpin structure. In certain embodiments, the first ITR and/or the second ITR forms a non-T-shaped hairpin structure. In some embodiments, the non-T-shaped hairpin structure comprises a U-shaped hairpin structure.

In some embodiments, an ITR in a nucleic acid molecule described herein may be a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some embodiments, the transcriptionally active element is a constitutive transcriptionally active element. Constitutive transcriptionally active elements provide an ongoing level of gene transcription and are preferred when it is desired that the transgene be expressed on an ongoing basis. In other embodiments, the transcriptionally active element is an inducible transcriptionally active element. Inducible transcriptionally active elements generally exhibit low activity in the absence of an inducer (or inducing condition) and are up-regulated in the presence of the inducer (or switch to an inducing condition). Inducible transcriptionally active elements may be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements can be incorporated into an ITR in a variety of ways. In some embodiments, a transcriptionally active element is incorporated 5′ to any portion of an ITR or 3′ to any portion of an ITR. In other embodiments, a transcriptionally active element of a transcriptionally-activated ITR lies between two ITR sequences. If the transcriptionally active element comprises two or more elements which must be spaced apart, those elements may alternate with portions of the ITR. In some embodiments, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these may be adjacent or spaced apart. In addition, protein binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Sp1 site, Inr region, CRE (cAMP regulatory element) site, ATF-1/CRE site, APBp box, APBa box, CArG box, CCAC box, or any other element involved in transcription as known in the art.

B. Therapeutic Proteins

Certain aspects of the present disclosure are directed to a nucleic acid molecule comprising a first ITR, a second ITR, and a genetic cassette encoding a target sequence, wherein the target sequence encodes a therapeutic protein. In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

Certain embodiments of the present disclosure are directed to a nucleic acid molecule comprising a first ITR, a second ITR, and a genetic cassette encoding a therapeutic protein, wherein the therapeutic protein comprises a clotting factor. In some embodiments, the clotting factor is selected from the group consisting of FI, FII, FIII, FIV, FV, FVI, FVII, FVIII, FIX, FX, FXI, FXII, FXIII, VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In one embodiment, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

In some embodiments, the nucleic acid molecule comprises a first ITR, a second ITR, and a genetic cassette encoding a target sequence, wherein the target sequence encodes a therapeutic protein, wherein the therapeutic protein comprises a factor VIII polypeptide. “Factor VIII,” abbreviated throughout the instant application as “FVIII,” as used herein, means functional FVIII polypeptide in its normal role in coagulation, unless otherwise specified. Thus, the term FVIII includes variant polypeptides that are functional. “A FVIII protein” is used interchangeably with FVIII polypeptide (or protein) or FVIII. Examples of the FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa.

The FVIII portion in the therapeutic protein used herein has FVIII activity. FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is used in conjunction with prothrombin time (PT), which measures the extrinsic pathway.

ROTEM analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The chromogenic assay mechanism is based on the principles of the blood coagulation cascade, where activated FVIII accelerates the conversion of Factor X into Factor Xa in the presence of activated Factor IX, phospholipids and calcium ions. The Factor Xa activity is assessed by hydrolysis of a p-nitroanilide (pNA) substrate specific to Factor Xa. The initial rate of release of p-nitroaniline measured at 405 nM is directly proportional to the Factor Xa activity and thus to the FVIII activity in the sample. The chromogenic assay is recommended by the FVIII and Factor IX Subcommittee of the Scientific and Standardization Committee (SSC) of the International Society on Thrombosis and Hemostatsis (ISTH). Since 1994, the chromogenic assay has also been the reference method of the European Pharmacopoeia for the assignment of FVIII concentrate potency.

In some embodiments, the genetic cassette comprises a nucleotide sequence encoding a FVIII polypeptide, wherein the nucleotide sequence is codon optimized. In some embodiments, the genetic cassette comprises a nucleotide sequence encoding a codon optimized FVIII driven by a mTTR promoter and synthetic intron. In some embodiments, the genetic cassette comprises a nucleotide sequence which is disclosed in International Application No. PCT/US2017/015879, which is incorporated by reference in its entirety. In some embodiments, the genetic cassette is a “hFVIIIco6XTEN” genetic cassette as described in PCT/US2017/015879. In some embodiments, the genetic cassette comprises SEQ ID NO: 32.

In some embodiments, the genetic cassette comprises codon optimized cDNA encoding B-domain deleted (BDD) codon-optimized human Factor VIII (BDDcoFVIII) fused with XTEN 144 peptide. In some embodiments, the genetic cassette comprises the nucleotide sequence set forth as SEQ ID NO: 9. In some embodiments, the genetic cassette comprises the nucleotide sequence set forth as SEQ ID NO: 33. In some embodiments, the genetic cassette comprises the nucleotide sequence set forth as SEQ ID NO: 14. In some embodiments, the genetic cassette has the nucleotide sequence of SEQ ID NO: 14. In some embodiments, the nucleic acid molecule comprises the nucleotide sequence of SEQ ID NO: 35.

In some embodiments, the genetic cassette comprises a nucleotide sequence encoding a codon optimized FVIII driven by a mTTR promoter. In some embodiments, the genetic cassette further comprises an A1MB2 enhancer element. In some embodiments, the genetic cassette further comprises a chimeric or synthetic intron. In some embodiments, the genetic cassette further comprises a a Woodchuck Posttranscriptional Regulatory Element (WPRE). In some embodiments, the genetic cassette further comprises a Bovine Growth Hormone Polyadenylation (bGHpA) signal.

In some embodiments, the present disclosure is directed to codon optimized nucleic acid molecules encoding a polypeptide with FVIII activity. In some embodiments, the polynucleotide encodes a full-length FVIII polypeptide. In other embodiments, the nucleic acid molecule encodes a B domain-deleted (BDD) FVIII polypeptide, wherein all or a portion of the B domain of FVIII is deleted.

In other embodiments, the nucleic acid molecules disclosed herein are further optimized by removal of one or more CpG motifs and/or the methylation of at least one CpG motif. As used herein, “CpG motif” refers to a dinucleotide sequence containing an unmethylated cytosine linked by a phosphate bond to a guanosine. The term “CpG motif” encompasses both methylated and unmethylated CpG dinucleotides. Unmethylated CpG motifs are common in nucleic acid of bacterial and viral origin (e.g., plasmid DNA) but are suppressed and largely methylated in vertebrate DNA. Thus, unmethylated CpG motifs stimulate the mammalian host to mount a rapid inflammatory response. Klinman, et al. (1996). PNAS 93:2879-2883. Exemplary methods of CpG removal are described in Yew, N. S., et al. (2002). Mol Ther. 5(6):731-738 and International Application No. PCT/US2001/010309. In some embodiments, the nucleic acid molecules disclosed herein have been modified to contain fewer CpG motifs (i.e., CpG reduced or CpG depleted). In one embodiment, the CpG motifs located within a codon triplet for a selected amino acid is changed to a codon triplet for the same amino acid lacking a CpG motif. In some embodiments, the nucleic acid molecules disclosed herein have been optimized to reduce innate immune response.

In one particular embodiment, the nucleic acid molecule encodes a polypeptide comprising an amino acid sequence having at least about 80%, at least about 85%, at least about 86%, at least about 87%, at least about 88%, at least about 89%, at least about 90%, at least about 95%, at least about 96%, at least about 97%, at least about 98%, or at least about 99% sequence identity to SEQ ID NO: 10 or a fragment thereof. In some embodiments, the nucleic acid molecule of the disclosure encodes a FVIII polypeptide comprising a signal peptide or a fragment thereof. In other embodiments, the nucleic acid molecule encodes a FVIII polypeptide which lacks a signal peptide. In some embodiments, the signal peptide comprises the amino acid sequence of SEQ ID NO: 11. In some embodiments, the signal peptide comprises amino acids 1-19 of SEQ ID NO: 10.

In some embodiments, the nucleic acid molecule comprises a first ITR, a second ITR, and a genetic cassette encoding a target sequence, wherein the target sequence encodes a therapeutic protein, and wherein the therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7. In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin B1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is foetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

C. Expression Control Sequences

In some embodiments, the nucleic acid molecule or vector of the disclosure further comprises at least one expression control sequence. For example, the isolated nucleic acid molecule of the disclosure can be operably linked to at least one expression control sequence. The expression control sequence can, for example, be a promoter sequence or promoter-enhancer combination.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. In certain embodiments, the promoter or other expression control sequence selectively enhances expression of the transgene in hepatocytes, sinusoidal cells, and/or endothelial cells. In one particular embodiment, the promoter or other expression control sequence selective enhances expression of the transgene in endothelial cells. In certain embodiments, the promoter or other expression control sequence selective enhances expression of the transgene in muscle cells, the central nervous system, the eye, the liver, the heart, or any combination thereof. Examples of liver specific promoters include, but are not limited to, a mouse transthyretin promoter (mTTR), a native human factor VIII promoter, a human alpha-1-antitrypsin promoter (hAAT), human albumin minimal promoter, and mouse albumin promoter. In some embodiments, the nucleic acid molecules disclosed herein comprise a mTTR promoter. The mTTR promoter is described in Costa et al. (1986) Mol. Cell. Biol. 6:4697. The FVIII promoter is described in Figueiredo and Brownlee, 1995, J. Biol. Chem. 270:11828-11838. In some embodiments, the promoter is selected from a liver specific promoter (e.g., a1-antitrypsin (AAT)), a muscle specific promoter (e.g., muscle creatine kinase (MCK), myosin heavy chain alpha (αMHC), myoglobin (MB), and desmin (DES)), a synthetic promoter (e.g., SPc5-12, 2R5Sc5-12, dMCK, and tMCK), or any combination thereof.

In some embodiments, the transgene expression is targeted to the liver. In certain embodiments, the transgene expression is targeted to hepatocytes. In other embodiment, the transgene expression is targeted to endothelial cells. In one particular embodiment, the transgene expression is targeted to any tissue that naturally expressed endogenous FVIII. In some embodiments, the transgene expression is targeted to the central nervous system. In certain embodiments, the transgene expression is targeted to neurons. In some embodiments, the transgene expression is targeted to afferent neurons. In some embodiments, the transgene expression is targeted to efferent neurons. In some embodiments, the transgene expression is targeted to interneurons. In some embodiments, the transgene expression is targeted to glial cells. In some embodiments, the transgene expression is targeted to astrocytes. In some embodiments, the transgene expression is targeted to oligodendrocytes. In some embodiments, the transgene expression is targeted to microglia. In some embodiments, the transgene expression is targeted to ependymal cells. In some embodiments, the transgene expression is targeted to Schwann cells. In some embodiments, the transgene expression is targeted to satellite cells. In some embodiments, the transgene expression is targeted to muscle tissue. In some embodiments, the transgene expression is targeted to smooth muscle. In some embodiments, the transgene expression is targeted to cardiac muscle. In some embodiments, the transgene expression is targeted to skeletal muscle. In some embodiments, the transgene expression is targeted to the eye. In some embodiments, the transgene expression is targeted to a photoreceptor cell. In some embodiments, the transgene expression is targeted to retinal ganglion cell.

Other promoters useful in the nucleic acid molecules disclosed herein include a mouse transthyretin promoter (mTTR), a native human FVIII promoter, a a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, a1-antitrypsin (AAT) promoter, muscle creatine kinase (MCK) promoter, myosin heavy chain alpha (αMHC) promoter, myoglobin (MB) promoter, desmin (DES) promoter, SPc5-12 promoter, 2R5Sc5-12 promoter, dMCK promoter, tMCK promoter, a phosphoglycerate kinase (PGK) promoter, or a human alpha-1-antitrypsin (A1AT) promoter, or any combinations thereof.

In some embodiments, the nucleic acid molecules disclosed herein comprise a transthyretin (TTR) promoter. In some embodiments, the promoter is a mouse transthyretin (mTTR) promoter. Non-limiting examples of mTTR promoters include the mTTR202 promoter, mTTR202opt promoter, and mTTR482 promoter, as disclosed in U.S. Publication No. US2019/0048362, which is incorporated by reference herein in its entirety. In some embodiments, the promoter is a liver-specific modified mouse transthyretin (mTTR) promoter. In some embodiments, the promoter is the liver-specific modified mouse transthyretin (mTTR) promoter mTTR482. Examples of mTTR482 promoters are described in Kyostio-Moore et al. (2016) Mol Ther Methods Clin Dev. 3:16006, and Nambiar B. et al. (2017) Hum Gene Ther Methods, 28(1):23-28. In some embodiments, the promoter is a liver-specific modified mouse transthyretin (mTTR) promoter comprising the nucleic acid sequence of SEQ ID NO: 16. In some embodiments, the tissue specific promoter is the human alpha-1-antitrypsin (A1AT) promoter. In some embodiments, the tissue specific promoter comprises the nucleotide sequence of SEQ ID NO: 36.

Expression levels can be further enhanced to achieve therapeutic efficacy using one or more enhancer elements. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al. (1992) J. Biol. Chem. 267:20765-20773; Rouet et al. (1995), Nucleic Acids Res. 23:395-404; Rouet et al (1998) Biochem. J. 334:577-584; III et al. (1997) Blood Coagulation Fibrinolysis 8:S23-S30). In some embodiments, the enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In some embodiments, the enhancer element comprises one or two modified prothrombin enhancers (pPrT2), one or two alpha 1-microbikunin enhancers (A1MB2), a modified mouse albumin enhancer (mEalb), a hepatitis B virus enhancer II (HE11), or a CRM8 enhancer. In some embodiments, the A1MB2 enhancer is the enhancer disclosed in International Application No. PCT/US2019/055917. In some embodiments, the enhancer element is A1MB2. In some embodiments, the enhancer element includes multiple copies of the A1MB2 enhancer sequence. In some embodiments, the A1MB2 enhancer is positioned 5′ to the nucleic acid sequence encoding the FVIII polypeptide. In some embodiments, the A1MB2 enhancer is positioned 5′ to the promoter sequence, such as the mTTR promoter. In some embodiments, the enhancer element is the A1MB2 enhancer comprising the nucleic acid sequence of SEQ ID NO: 15.

In some embodiments, the nucleic acid molecules disclosed herein comprise an intron or intronic sequence. In some embodiments, the intronic sequence is a naturally occurring intronic sequence. In some embodiments, the intronic sequence is a synthetic sequence. In some embodiments, the intronic sequence is derived from a naturally occurring intronic sequence. In some embodiments, the intronic sequence is a hybrid synthetic intron or chimeric intron. In some embodiments, the intronic sequence is a chimeric intron that consists of chicken beta-actin/rabbit beta-globin intron and has been modified to eliminate five existing ATG sequences to reduce false translation starts. In some embodiments, the chimeric intron comprises the nucleic acid sequence of SEQ ID NO: 17. In some embodiments, the intronic sequence is positioned 5′ to the nucleic acid sequence encoding the FVIII polypeptide. In some embodiments, the chimeric intron is positioned 5′ to a promoter sequence, such as the mTTR promoter.

In some embodiments, the nucleic acid molecules disclosed herein comprise a post-transcriptional regulatory element. In certain embodiments, the post-transcriptional regulatory element comprises a mutated woodchuck hepatitis virus regulatory element (WPRE). WPRE is believed to enhance the expression of viral vector-delivered transgenes. Examples of WPRE are described in Zufferey et al. (1999) J Virol., 73(4):2886-2892; Loeb et al. (1999) Hum Gene Ther. 10(14):2295-2305. In some embodiments, the WPRE is positioned 3′ to the nucleic acid sequence encoding the FVIII polypeptide. In some embodiments, the WPRE comprises the nucleic acid sequence of SEQ ID NO: 18.

In some embodiments, the nucleic acid molecules disclosed herein comprise a transcription terminator. In some embodiments, the transcription terminator is a polyadenylation (poly(A)) sequence. Non-limiting examples of transcriptional terminators include those derived from the bovine growth hormone polyadenylation signal (BGHpA), the Simian virus 40 polyadenylation signal (SV40 pA), or a synthetic polyadenylation signal. In one embodiment, the 3′UTR poly(A) tail comprises an actin poly(A) site. In one embodiment, the 3′UTR poly(A) tail comprises a hemoglobin poly(A) site. In some embodiments, the transcriptional terminator is BGHpA. Examples of BGHpA transcriptional terminators are described in Woychik et al. (1984) PNAS 81:3944-3948. In some embodiments, the transcriptional terminator is positioned at the 3′ end of the genetic cassette encoding the nucleic acid sequence encoding the FVIII polypeptide. In some embodiments, the transcriptional terminator is a BGHpA comprising the nucleic acid sequence of SEQ ID NO: 19.

In some embodiments, the nucleic acid molecule disclosed herein comprises one or more DNA nuclear targeting sequences (DTSs). A DTS promotes translocation of DNA molecules containing such sequences into the nucleus. In certain embodiments, the DTS comprises an SV40 enhancer sequence. In certain embodiments, the DTS comprises a c-Myc enhancer sequence. In some embodiments, the nucleic acid molecule comprises DTSs that are located between the first ITR and the second ITR. In some embodiments, the nucleic acid molecule comprises a DTS located 3′ to the first ITR and 5′ to the transgene (e.g., FVIII protein). In some embodiments, the nucleic acid molecule comprises a DTS located 3′ to the transgene and 5′ to the second ITR on the nucleic acid molecule.

In some embodiments, the nucleic acid molecule disclosed herein comprises a toll-like receptor 9 (TLR9) inhibition sequence. Exemplary TLR9 inhibition sequences are described in, e.g., Trieu et al. (2006) Crit Rev Immunol. 26(6):527-44; Ashman et al. Int'l Immunology 23(3): 203-14.

In some embodiments, the nucleic acid molecule disclosed herein comprises a nucleic acid sequence encoding a nonstructural protein of HBoV1. “Nonstructural proteins” refers to any of six proteins, namely, NS1, NS1-70, NS2, NS3, NS4, and NP1, which are expressed by HBoV1. Nonstructural proteins are expressed by mRNA transcripts generated through alternative splicing and the polyadenylation of a single viral pre-mRNA. The NS1 to NS4 proteins are encoded in different regions of the same open reading frame (ORF). NS1 binds to the HBoV1 replication origin and presumably nicks single-stranded DNA (ssDNA) of the origin during rolling-hairpin replication. NS1 plays an important role in the expression of HBoV1 ITR-mediated vector production in eukaryotic cells. In one embodiment, expression constructs were generated which express HBoV1 NS1. In some embodiments, the nucleic acid molecule disclosed herein encodes a nonstructural protein described in Shen et al. (2015) J Virology 89(19): 10097-10109.

In some embodiments, the nucleic acid molecule comprises a microRNA (miRNA) binding site. In one embodiment, the miRNA binding site is a miRNA binding site for miR-142-3p. In other embodiments, the miRNA binding site is a miRNA binding site described by Rennie et al. (2016) RNA Biol. 13(6):554-560.

Production of ceDNA in Baculoviruses

Baculoviruses are the most prominent viruses that infect insects. Over 500 baculovirus isolates have been identified, the majority of which originated in insects of the order Lepidoptera. The two most common isolates are Autographa californica multiple nucleopolyhedrovirus (AcMNPV) and Bombyx mori nucleopolyhedrovirus (BmNPV). Among expression vectors, baculovirus stands out because of their outsized genetic cargo capacity—up to several 10s of kb, with some reports up to 100 kb. This transgene capacity has been used for the production of recombinant AAV vectors (up to 38 kb expression cassettes). However, when producing viral or non-viral vector for gene therapy, several baculovirus expression vectors are often required to be infected into insect host cells. The generation of each of the baculovirus expression vectors is time consuming, and drives up the cost of production, representing a significant disadvantage of most baculovirus expression vector systems. However, a new versatile baculovirus shuttle vector (bacmid) was generated specifically designed to accommodate multiple transgenes that could be accomplished with the existing bacmid tools. This versatile bacmid (called “BIVVBac”) could also be used for rAAV vector production for in vivo gene therapy, as well as for the production of any desired protein, e.g., a recombinant protein. This bacmid expression system is further described in U.S. Patent Application No. 63/069,073, hereby incorporated by reference in its entirety.

In certain embodiments, the disclosed nucleic acid molecules are produced using a baculovirus expression vector system comprising the “BIVVBac” recombinant bacmid. In certain embodiments, the BIVVBac is a genetically modified AcMNPV that comprises at least two foreign sequence insertion sites. A baculovirus expression vector system comprising a bacmid that comprises at least two foreign sequence insertion sites allows for the reduction in total number of baculovirus expression vectors that need to be generated.

In certain embodiments, the BIVVBac comprises a first and a second foreign sequence insertion site. The first and the second foreign sequence insertion sites may be different, utilizing different machinery to drive insertion of a foreign sequence (e.g., heterologous sequence, heterologous gene). Insertion of a foreign sequence may be driven by any method known in the art. For example, a foreign sequence may be inserted by transposition or site-specific recombination. A foreign sequence insertion site may be designed to be comprised within a reporter gene such that upon insertion of the foreign sequence, the reporter gene becomes disrupted. Disruption of the reporter gene may aid in the identification of bacmid clones having a foreign sequence inserted therein. In such embodiments, the foreign sequence insertion site is fused in-frame with the reporter gene, or the reporter gene is fused in-frame with the foreign sequence insertion site.

In certain embodiments, the first foreign sequence insertion site allows for the insertion of a foreign sequence via transposition. In certain embodiments, the first foreign sequence insertion site comprises a preferential target site for the insertion of a transposon. In certain embodiments, the first foreign sequence insertion site is a preferential target site for the insertion of a transposon. In certain embodiments, the first foreign sequence insertion site is a preferential target site that is an attachment site for a bacterial transposon. Suitable bacterial transposons and their corresponding attachment sites are known to those of skill in the art. For example, the transposon Tn7 is known for its ability to transpose to a specific site of a bacterial chromosome (attTn7) at a high frequency. Accordingly, in certain embodiments, the first foreign sequence insertion site is a preferential target site that is an attachment site for a Tn7 transposon (e.g., attTn7). In some embodiments, the first foreign sequence insertion site is a preferential target site that is an attachment site for a mini-Tn7 transposon (e.g., mini-attTn7, the minimal DNA sequence required for recognition by Tn7 transposition factors and insertion of a Tn7 transposon).

In certain embodiments, the second foreign sequence insertion site allows for the insertion of a foreign sequence via site-specific recombination. In certain embodiments, the second foreign sequence insertion site comprises a preferential target site capable of mediating a site-specific recombination event. Various site-specific recombinase technologies are known to those of skill in the art. For example, the Cre-loxP system mediates site-specific recombination via Cre recombinase which is capable of recognizing 34 base pair DNA sequences called loxP sites. Accordingly, the second foreign sequence insertion site is a preferential target site for Cre mediated recombination. In certain embodiments, the second foreign sequence insertion site is a preferential target site comprising a loxP site or a variant thereof capable of being recognized by Cre recombinase.

In some embodiments, the recombinant bacmid comprises a variant VP80 gene, such that the bacmid exhibits reduced expression of its encoded protein. For example, disclosed herein is a baculovirus DNA backbone comprising an inactivated VP80 gene due to an insertion and/or deletion in the VP80 gene locus. In some embodiments, the recombinant bacmid comprises a bacmid disclosed in U.S. Patent Application No. U.S. 63/069,115.

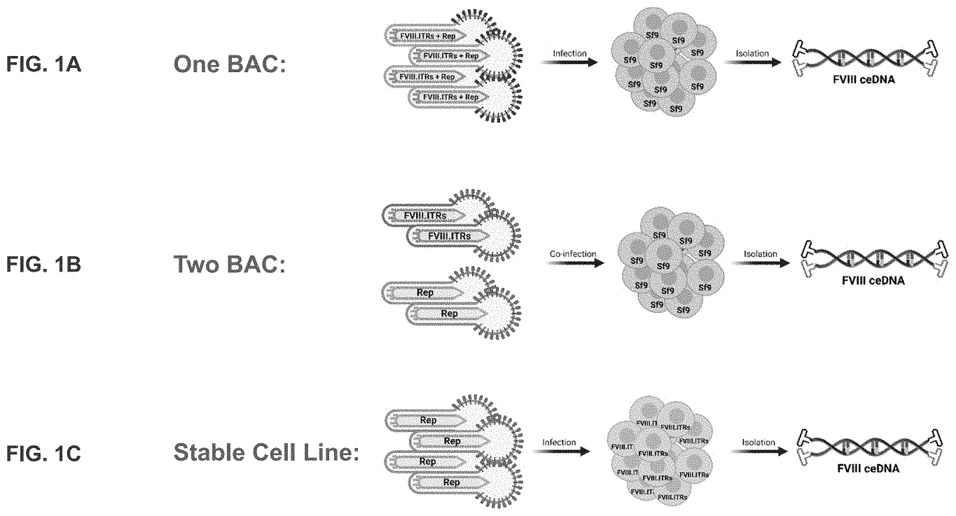

In certain embodiments, ceDNA is produced using a single baculovirus expression vector. In this “OneBAC” approach, a single baculovirus expression vector (e.g., BIVVBac) encodes all essential elements required for ceDNA production in the baculovirus system and could potentially be used in any baculovirus permissive cell lines for ceDNA production. This approach is depicted in FIG. 1A.

In certain embodiments, ceDNA is produced using multiple multiple baculovirus expression vectors. In this “TwoBAC” approach, essential elements required for ceDNA production are inserted into two different baculoviruses (e.g., two BIVVBac bacmids) and could potentially be used for co-infection in any cell lines permissive for baculovirus infection. This approach is depicted in FIG. 1B.

In certain embodiments, ceDNA is produced by a stable cell line. In this approach, the essential elements required for ceDNA production are inserted in both components of the baculovirus system. This approach is depicted in FIG. 1C. A stable cell line can be generated by stably integrating a protein encoding sequence under the control of a baculovirus gene promoter (e.g., a baculovirus constitutive gene promoter). In certain embodiments, the stable cell line is a stable insect cell line.

Methods for stable integration of nucleic acids into a variety of host cell lines are known in the art. For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A. C. et al. (2000) Proc. Natl. Acad. Sci. 97:5995-6000).

The disclosure also provides a polypeptide encoded by a nucleic acid molecule of the disclosure. In some embodiments, the polypeptide of the disclosure is encoded by a vector comprising the isolated nucleic molecules disclosed herein. In yet other embodiments, the polypeptide of the disclosure is produced by a host cell comprising the isolated nucleic molecules disclosed herein.

### Host Cells

The disclosure also provides a host cell comprising a nucleic acid molecule or vector of the disclosure. As used herein, the term “transformation” shall be used in a broad sense to refer to the introduction of DNA into a recipient host cell that changes the genotype and consequently results in a change in the recipient cell.

“Host cells” refers to cells that have been transformed with vectors constructed using recombinant DNA techniques and encoding at least one heterologous gene. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3x63-Ag8.653 (mouse myeloma), BFA-1c1BPT (bovine endothelial cells), RAJI (human lymphocyte), PER.C6©, NS0, CAP, BHK21, and HEK 293 (human kidney). In one particular embodiment, the host cell is selected from the group consisting of: a CHO cell, a HEK293 cell, a BHK21 cell, a PER.C6© cell, a NS0 cell, a CAP cell and any combination thereof. In some embodiments, the host cells of the present disclosure are of insect origin. In one particular embodiment, the host cells are SF9 cells. Host cell lines are typically available from commercial services, the American Tissue Culture Collection, or from published literature.

Introduction of the nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

Host cells comprising the isolated nucleic acid molecules or vectors of the disclosure are grown in an appropriate growth medium. As used herein, the term “appropriate growth medium” means medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, Calif.). In another embodiment, the medium is CD17 (Invitrogen, Carlsbad, Calif.). Selection of a medium appropriate for the particular cell line used is within the level of those ordinary skilled in the art.

Aspects of the present disclosure provide a method of cloning a nucleic acid molecule described herein, comprising inserting a nucleic acid molecule capable of complex secondary structures into a suitable vector, and introducing the resulting vector into a suitable bacterial host strain. As known in the art, complex secondary structures (e.g., long palindromic regions) of nucleic acids may be unstable and difficult to clone in bacterial host strains. For example, nucleic acid molecules comprising a first ITR and a second ITR (e.g., non-AAV parvoviral ITRs, e.g., HBoV1 ITRs) of the present disclosure may be difficult to clone using conventional methodologies. Long DNA plindromes inhibit DNA replication and are unstable in the genomes of E. coli, Bacillus, Streptococcus, Streptomyces, S. cerevisiae, mice, and humans. These effects result from the formation of hairpin or cruciform structures by intrastrand base pairing. In E. coli the inhibition of DNA replication can be significantly overcome in SbcC or SbcD mutants. SbcD is the nuclease subunit, and SbcC is the ATPase subunit of the SbcCD complex. The E. coli SbcCD complex is an exonuclease complex responsible for preventing the replication of long palindromes. The SbcCD complex is a nuclear with ATP-dependent double-stranded DNA exonuclease activity and ATP-independent single-stranded DNA endonuclease activity. SbcCD may recognize DNA plaindromes and collapse replication forks by attacking hairpin structures that arise.

In certain embodiments, a suitable bacterial host strain is incapable of resolving cruciform DNA structures. In certain embodiments, a suitable bacterial host strain comprises a disruption in the SbcCD complex. In some embodiments, the disruption in the SbcCD complex comprises a genetic disruption in the SbcC gene and/or SbcD gene. In certain embodiments, the disruption in the SbcCD complex comprises a genetic disruption in the SbcC gene. Various bacterial host strains that comprise a genetic disruption in the SbcC gene are known in the art. For example, without limitation, the bacterial host strain PMC103 comprises the genotype sbcC, recD, mcrA, ΔmcrBCF; the bacterial host strain PMC107 comprises the genotype recBC, recJ, sbcBC, mcrA, ΔmcrBCF; and the bacterial host strain SURE comprises the genotype recB, recJ, sbcC, mcrA, ΔmcrBCF, umuC, uvrC. Accordingly, in some embodiments a method of cloning a nucleic acid molecule described herein comprises inserting a nucleic acid molecule capable of complex secondary structures into a suitable vector, and introducing the resulting vector into host strain PMC103, PMC107, or SURE. In certain embodiments, the method of cloning a nucleic acid molecule described herein comprises inserting a nucleic acid molecule capable of complex secondary structures into a suitable vector and introducing the resulting vector into host strain PMC103.

Suitable vectors are known in the art. In certain embodiments, a suitable vector for use in a cloning methodology of the present disclosure is a low copy vector. In certain embodiments, a suitable vector for use in a cloning methodology of the present disclosure is pBR322.

Accordingly, the present disclosure provides a method of cloning a nucleic acid molecule, comprising inserting a nucleic acid molecule capable of complex secondary structures into a suitable vector, and introducing the resulting vector into a bacterial host strain comprising a disruption in the SbcCD complex, wherein the nucleic acid molecule comprises a first inverted terminal repeat (ITR) and a second ITR, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs. 1 or 2 or a functional derivative thereof.

### Production of FVIII Polypeptides

The disclosure also provides a polypeptide encoded by a nucleic acid molecule of the disclosure. In some embodiments, the polypeptide of the disclosure is encoded by a vector comprising the isolated nucleic molecules disclosed herein. In yet other embodiments, the polypeptide of the disclosure is produced by a host cell comprising the isolated nucleic molecules of the disclosure.

A variety of methods are available for recombinantly producing a FVIII protein from the optimized nucleic acid molecule of the disclosure. A polynucleotide of the desired sequence can be produced by de novo solid-phase DNA synthesis or by PCR mutagenesis of an earlier prepared polynucleotide. Oligonucleotide-mediated mutagenesis is one method for preparing a substitution, insertion, deletion, or alteration (e.g., altered codon) in a nucleotide sequence. For example, the starting DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide of the disclosure.

For recombinant protein production, an optimized polynucleotide sequence of the disclosure encoding the FVIII protein is inserted into an appropriate expression vehicle, i.e., a vector which contains the necessary elements for the transcription and translation of the inserted coding sequence, or in the case of an RNA viral vector, the necessary elements for replication and translation.

The polynucleotide sequence of the disclosure is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14: 725) and electroporation (Neumann et al. 1982, EMBO, J. 1: 841). A variety of host-expression vector systems can be utilized to express the FVIII proteins described herein in eukaryotic cells. In one embodiment, the eukaryotic cell is an animal cell, including mammalian cells (e.g., HEK293 cells, PER.C6©, CHO, BHK, Cos, HeLa cells). A polynucleotide sequence of the disclosure can also code for a signal sequence that will permit the FVIII protein to be secreted. One skilled in the art will understand that while the FVIII protein is translated the signal sequence is cleaved by the cell to form the mature protein. Various signal sequences are known in the art, e.g., native factor VII signal sequence, native factor IX signal sequence and the mouse IgK light chain signal sequence. Alternatively, where a signal sequence is not included the FVIII protein can be recovered by lysing the cells.

The FVIII protein of the disclosure can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad. Sci. USA 78: 6376; McKnight et al. 1983, Cell 34: 335; Brinster et al. 1983, Nature 306: 332; Ritchie et al. 1984, Nature 312: 517; Baldassarre et al. 2003, Theriogenology 59: 831; Robl et al. 2003, Theriogenology 59: 107; Malassagne et al. 2003, Xenotransplantation 10 (3): 267).

The expression vectors can encode for tags that permit for easy purification or identification of the recombinantly produced protein. Examples include, but are not limited to, vector pUR278 (Ruther et al. 1983, EMBO J. 2: 1791) in which the FVIII protein described herein coding sequence can be ligated into the vector in frame with the lac Z coding region so that a hybrid protein is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites (e.g., PreCission Protease (Pharmacia, Peapack, N.J.)) for easy removal of the tag after purification.

For the purposes of this disclosure, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed or introduced into the same cell by cotransformation.

An example of a vector useful for expressing an optimized FVIII sequence is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high-level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 μg/cell/day. Other exemplary vector systems are disclosed e.g., in U.S. Pat. No. 6,413,777.

In other embodiments the polypeptides of the disclosure of the instant disclosure can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is also incorporated herein.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art, as discussed above. The transformed cells are grown under conditions appropriate to the production of the FVIII polypeptide and assayed for FVIII polypeptide synthesis. Exemplary assay techniques include enzyme-linked immunosorbent assay (ELISA), radioimmunoassay (RIA), or fluorescence-activated cell sorter analysis (FACS), immunohistochemistry and the like.

In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

The host cell line used for protein expression is preferably of mammalian origin; most preferably of human or mouse origin, as the isolated nucleic acids of the disclosure have been optimized for expression in human cells. Exemplary host cell lines have been described above. In one embodiment of the method to produce a polypeptide with FVIII activity, the host cell is a HEK293 cell. In another embodiment of the method to produce a polypeptide with FVIII activity, the host cell is a CHO cell.

Genes encoding the polypeptides of the disclosure can also be expressed in non-mammalian cells such as bacteria or yeast or plant cells. In this regard it will be appreciated that various unicellular non-mammalian microorganisms such as bacteria can also be transformed i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

Alternatively, optimized nucleotide sequences of the disclosure can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein. An affinity tag sequence (e.g. a His(6) tag) can optionally be attached or included within the polypeptide sequence to facilitate downstream purification.

Once expressed, the FVIII protein can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)). Substantially pure proteins of at least about 90 to 95% homogeneity are preferred for pharmaceutical uses, with 98 to 99% or more homogeneity being most preferred.

Pharmaceutical Compositions

Compositions containing an isolated nucleic acid molecule, a polypeptide having FVIII activity encoded by the nucleic acid molecule, a vector, or a host cell of the present disclosure can contain a suitable pharmaceutically acceptable carrier. For example, they can contain excipients and/or auxiliaries that facilitate processing of the active compounds into preparations designed for delivery to the site of action.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the chimeric protein of the disclosure is formulated with another clotting factor, or a variant, fragment, analogue, or derivative thereof. For example, the clotting factor includes, but is not limited to, factor V, factor VII, factor VIII, factor IX, factor X, factor XI, factor XII, factor Xll, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In one embodiment, a pharmaceutical composition comprises a polypeptide having Factor VIII activity, an optimized nucleic acid molecule encoding the polypeptide having Factor VIII activity, the vector comprising the nucleic acid molecule, or the host cell comprising the vector, and a pharmaceutically acceptable carrier. In some embodiments, the composition is administered by a route selected from the group consisting of topical administration, intraocular administration, parenteral administration, intrathecal administration, subdural administration and oral administration. The parenteral administration can be intravenous or subcutaneous administration.

Methods of Treatment

In some aspects, the present disclosure is directed to methods of treating a disease or condition in a subject in need thereof, comprising administering a nucleic acid molecule, a vector, a polypeptide, or a pharmaceutical composition disclosed herein.

In some embodiments, the disclosure is directed to methods of treating a bleeding disorder. In some embodiments, the disclosure is directed to methods of treating hemophilia A.

The isolated nucleic acid molecule, vector, or polypeptide can be administered intravenously, subcutaneously, intramuscularly, or via any mucosal surface, e.g., orally, sublingually, buccally, sublingually, nasally, rectally, vaginally or via pulmonary route. The clotting factor protein can be implanted within or linked to a biopolymer solid support that allows for the slow release of the chimeric protein to the desired site.

In one embodiment, the route of administration of the isolated nucleic acid molecule, vector, or polypeptide is parenteral. The term parenteral as used herein includes intravenous, intraarterial, intraperitoneal, intramuscular, subcutaneous, rectal or vaginal administration. In some embodiments, the isolated nucleic acid molecule, vector, or polypeptide is adminstered intraveneously. While all these forms of administration are clearly contemplated as being within the scope of the disclosure, a form for administration would be a solution for injection, in particular for intravenous or intraarterial injection or drip.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The nucleic acid molecule, vector, or polypeptides of the disclosure can optionally be administered in combination with other agents that are effective in treating the disorder or condition in need of treatment (e.g., prophylactic or therapeutic).

As used herein, the administration of isolated nucleic acid molecules, vectors, or polypeptides of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the isolated nucleic acid molecule, vector, or polypeptide of the instant disclosure can be used in conjunction or combination with an agent or agents (e.g., to provide a combined therapeutic regimen). Exemplary agents with which a polypeptide or polynucleotide of the disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polynucleotides or polypeptides of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996). In another embodiment, an amount of such an agent consistent with the standard of care is administered.

In one embodiment, also disclosed herein is a kit, comprising the nucleic acid molecule disclosed herein and instructions for administering the nucleic acid molecule to a subject in need thereof. In another embodiment, disclosed herein is a baculovirus system for production of the nucleic acid molecule provided herein. The nucleic acid molecule is produced in insect cells. In another embodiment, a nanoparticle delivery system for expression constructs is provided. The expression construct comprises the nucleic acid molecule disclosed herein.

### Gene Therapy

In some embodiments, the nucleic acid molecule disclosed herein is used in gene therapy. The optimized FVIII nucleic acid molecules disclosed herein can be used in any context where expression of FVIII is required. In some embodiments, the nucleic acid molecules comprise the nucleotide sequence of SEQ ID NO: 2. In some embodiments, the nucleic acid molecules comprise the nucleotide sequence of SEQ ID NO: 1.

For example, somatic gene therapy has been explored as a possible treatment for hemophilia A. Gene therapy is a particularly appealing treatment for hemophilia because of its potential to cure the disease through continuous endogenous production of FVIII following a single administration of vector. Hemophilia A is well suited for a gene replacement approach because its clinical manifestations are entirely attributable to the lack of a single gene product (FVIII) that circulates in minute amounts (200 ng/ml) in the plasma.

In one aspect, the nucleic acid molecule described herein may be used in AAV gene therapy. AAV is able to infect a number of mammalian cells. See, e.g., Tratschin et al. (1985) Mol. Cell Biol. 5:3251-3260 and Grimm et al. (1999) Hum. Gene Ther. 10:2445-2450. A rAAV vector carries a nucleic acid sequence encoding a gene of interest, or fragment thereof, under the control of regulatory sequences which direct expression of the product of the gene in cells. In some embodiments, the rAAV is formulated with a carrier and additional components suitable for administration.

In another aspect, the nucleic acid molecule described herein may be used in lentiviral gene therapy. Lentiviruses are RNA viruses wherein the viral genome is RNA. When a host cell is infected with a lentivirus, the genomic RNA is reverse transcribed into a DNA intermediate which is integrated very efficiently into the chromosomal DNA of infected cells. In some embodiments, the lentivirus is formulated with a carrier and additional components suitable for administration. In another aspect, the nucleic acid molecule described herein may be used in adenoviral therapy. A review of the use of adenovirus for gene therapy can be found e.g. in Wold et al. (2013) Curr Gene Ther. 13(6):421-33). In another aspect, the nucleic acid molecule described herein may be used in non-viral gene therapy. An optimized FVIII protein of the disclosure can be produced in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the iliopsoas sheath would be therapeutically beneficial. In one embodiment, the bleeding disease or disorder is hemophilia. In another embodiment, the bleeding disease or disorder is hemophilia A. This involves administration of an optimized FVIII encoding nucleic acid operably linked to suitable expression control sequences. In certain embodiment, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. In other embodiments, an adenoviral vector has a deletion in its E1 gene or E3 gene. In other embodiments, the sequences are incorporated into a non-viral vector known to those skilled in the art.

In another aspect, the nucleic acid molecules disclosed herein are used for specific alteration of the genetic information (e.g., genome) of living organisms. As used herein, the term “alteration” or “alteration of genetic information” refers to any change in the genome of a cell. In the context of treating genetic disorders, alterations may include, but are not limited to, insertion, deletion and/or correction.

In some aspects, alterations may also include a gene knock-in, knock-out or knock down. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a cDNA encoding the wild-type protein may be inserted into the genome of a cell carrying a mutant gene. Knock-in strategies need not replace the defective gene, in whole or in part. In some cases, a knock-in strategy may further involve substitution of an existing sequence with the provided sequence, e.g., substitution of a mutant allele with a wildtype copy. The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

In some embodiments, the nucleic acid sequences disclosed herein are used for genome editing. Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating single-strand or double strand DNA breaks at particular locations within the genome. Such breaks can be and regularly are repaired by natural, endogenous cellular processes, such as homology-directed repair (HDR) and non-homologous end joining (NHEJ), as recently reviewed in Cox et al. (2015). Nature Medicine 21(2): 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process, see, e.g., Cho and Greenberg (2015). Nature 518, 174-76. In some instances, it may be possible to predict likely repair outcomes based on analysis of potential microhomologies at the site of the DNA break.

Each of these genome editing mechanisms can be used to create desired genomic alterations. A step in the genome editing process can be to create one or two DNA breaks, the latter as double-strand breaks or as two single-stranded breaks, in the target locus as near the site of intended mutation. This can be achieved via the use of site-directed polypeptides, such as the CRISPR endonuclease system and others.

In another aspect, the nucleic acid molecule described herein may be used in lipid nanoparticle (LNP)-mediated delivery of FVIII ceDNA. Lipid nanoparticles formed from cationic lipids with other lipid components, such as neutral lipids, cholesterol, PEG, PEGylated lipids, and oligonucleotides have been used to block degradation of nucleic acids in plasma and facilitate the cellular uptake of oligonucleotides. Such lipid nanoparticles may be used to deliver the nucleic acid molecule described herein to subjects.

The disclosure provides a method of increasing expression of a polypeptide with FVIII activity in a subject comprising administering the isolated nucleic acid molecule of the disclosure to a subject in need thereof, wherein the expression of the polypeptide is increased relative to a reference nucleic acid molecule comprising SEQ ID NO: 6. The disclosure also provides a method of increasing expression of a polypeptide with FVIII activity in a subject comprising administering a vector of the disclosure to a subject in need thereof, wherein the expression of the polypeptide is increased relative to a vector comprising a reference nucleic acid molecule.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

### EXAMPLES

Having provided the foregoing disclosure, a further understanding can be obtained by reference to the examples provided herein. These examples are for purposes of illustration only and are not intended to be limiting.

### Example 1: Approaches to ceDNA Production

In the baculovirus-insect cell system, recombinant BEV delivers the gene of interest under a strong promoter and provides transcriptional complex essential for the virus replication in insect cells. This system provides the flexibility of inserting transgene of interest either in the baculovirus genome and/or insect cell genome in a form of stable cell line. Leveraging these advantages of the baculovirus-insect cell system, three different approaches of ceDNA production were designed to provide the wide selection according to the ease of scalability.

1. OneBAC

To investigate the use of a OneBac approach for transgene expression, the optimized FVIIIXTEN expression cassette was inserted with parvoviral ITRs at the mini-attTn7 site in the Polyhedrin locus in BIVVBac through Tn7 transposition and in the same backbone, the ITR-specific Replication (Rep) gene expression cassette was inserted at the LoxP site in the EGT locus through Cre-LoxP recombination. The recombinant BEV was then generated and used for infection in Sf9 cells to produce FVIIIXTEN ceDNA, as depicted in FIG. 1A. Different promoters for controlling the Rep expression levels were used to prove the concept of the OneBac approach for ceDNA production, as described below.

2. TwoBAC:

To investigate the use of a TwoBac approach for transgene expression, the optimized FVIIIXTEN expression cassette was inserted with parvoviral ITRs and the ITR-specific Replication (Rep) gene expression cassette at the mini-attTn7 site in the polyhedrin locus in two different BIVVBac bacmids through Tn7 transposition. The recombinant BEVs were then generated and used for co-infection in Sf9 cells to produce FVIIIXTEN ceDNA, as depicted in FIG. 1B. Challenges associated with the TwoBAC approach were investigated using different ratios of multiplicity of infection (MOIs) of two baculoviruses and fine-tuning of the Rep expression levels to obtain reproducible ceDNA productivity, as described in the following experiments.

3. Stable Cell Line:

To investigate the use of a stable cell line approach for transgene expression, a stable cell line was generated with the optimized FVIIIXTEN expression cassette with parvoviral ITRs. A recombinant bacmid was also generated by inserting the ITR-specific Replication (Rep) gene expression cassette at the mini-attTn7 site in the polyhedrin locus in BIVVBac bacmid through Tn7 transposition. The recombinant Rep.BEV was then generated and used for infection in the FVIIIXTEN stable cell line to produce FVIIIXTEN ceDNA, as depicted in FIG. 1C. Challenges associated with the Stable Cell Line approach were investigated by enriching FVIIIXTEN transformers through FACS cell sorting using GFP as a proxy to expedite the process of generating stable cell line, as described in the following experiments.

### Example 2: FVIIIXTEN HBoV1 ITRs Expression Construct

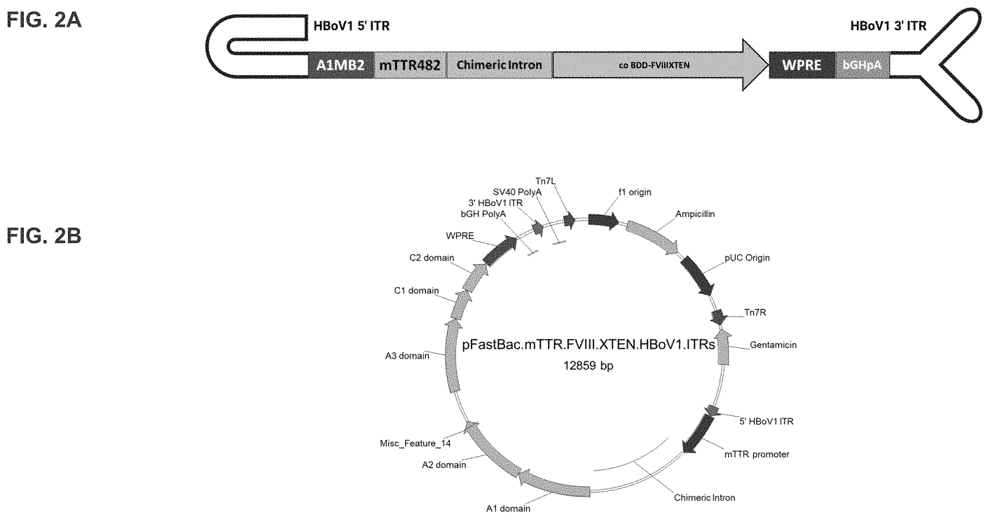

Human Bocavirus 1 (HBoV1), an autonomous parvovirus, is a helper virus supporting replication of wild-type adeno-associated virus 2 (AAV2). The use of AAV as well as non-AAV parvoviral ITRs for FVIIIXTEN ceDNA production has been demonstrated in the baculovirus system (see, e.g., U.S. Patent Application No. 63/069,073). HBoV1 ITRs have unique size and form in comparison with other parvoviral ITRs. The 5′ (REH) ITR of HBoV1 is 140 bp long (SEQ ID NO: 1) and forms “U” shape hairpin with perfect base pairing, whereas the 3′ (LEH) ITR is 200 bp long (SEQ ID NO: 2) and forms a loop with a three-way junction, which makes it an asymmetric in nature distinct from terminal regions of other parvoviral ITRs (FIG. 2A).

HboV1 ITRs were investigated for use in production of FVIIIXTEN ceDNA. It was hypothesized that the asymmetric ITRs may enhance long-term persistent expression by stabilizing the transgene. To test this hypothesis, a DNA construct was synthesized comprising a B-domain deleted (BDD) codon-optimized human Factor VIII (BDDcoFVIII) comprising an XTEN 144 peptide (FVIIIXTEN) under the regulation of liver-specific modified mouse transthyretin (mTTR) promoter (mTTR482) with enhancer element (A1MB2), hybrid synthetic intron (Chimeric Intron), the Woodchuck Posttranscriptional Regulatory Element (WPRE), the Bovine Growth Hormone Polyadenylation (bGHpA) signal, and flanking human HBoV1 5′ and 3′ ITRs through GenScript® (Piscataway, N.J.)® to generate the nucleic acid sequence set forth as SEQ ID NO: 3 (FIG. 2A). This synthetic DNA was cloned into pFastBac1 (Invitrogen) vector to generate pFastBac.mTTR.FVIIIXTEN.HBoV1.ITRs transfer vector (FIG. 2B). This vector was then transformed into BIVVBac^{DH10B} E. coli to produce a recombinant BEV, AcBIVVBac.Polh.GPV.Rep^{Tn7}, as described below.

### Example 3: HBoV1 NS1 (Nonstructural) Expression Constructs

HBoV1 NS1 Tn7 Transfer Vectors

HBoV1 is known to express five nonstructural proteins, namely, NS1, NS2, NS3, NS4, and NP1, by mRNA transcripts generated through alternative splicing and the polyadenylation of a single viral pre-mRNA. The NS1 to NS4 proteins are encoded in different regions of the same open reading frame (ORF). NS1 consists of an origin-binding/endonuclease domain (OBD), a helicase domain, and a putative transactivation domain (TAD) in the N terminus, middle, and C terminus, respectively. NS1 binds to the HBoV1 replication origin and presumably nicks single-stranded DNA (ssDNA) of the origin during rolling-hairpin replication.

To investigate the role of NS1 in ITR-mediated vector production in eukaryotic cells and to ‘rescue’ an HBoV1 ITR-flanked FVIIIXTEN ceDNA vector genome from Sf9 cells, a HBoV1 NS1 expression construct was generated and inserted into the BIVVBac to produce recombinant BEV expressing HBoV1 NS1 in Sf9 cells.

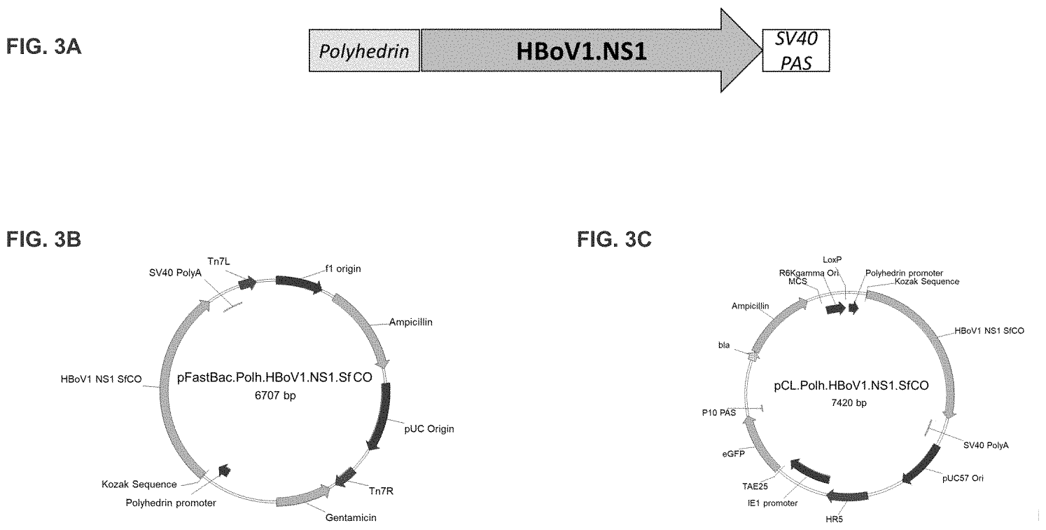

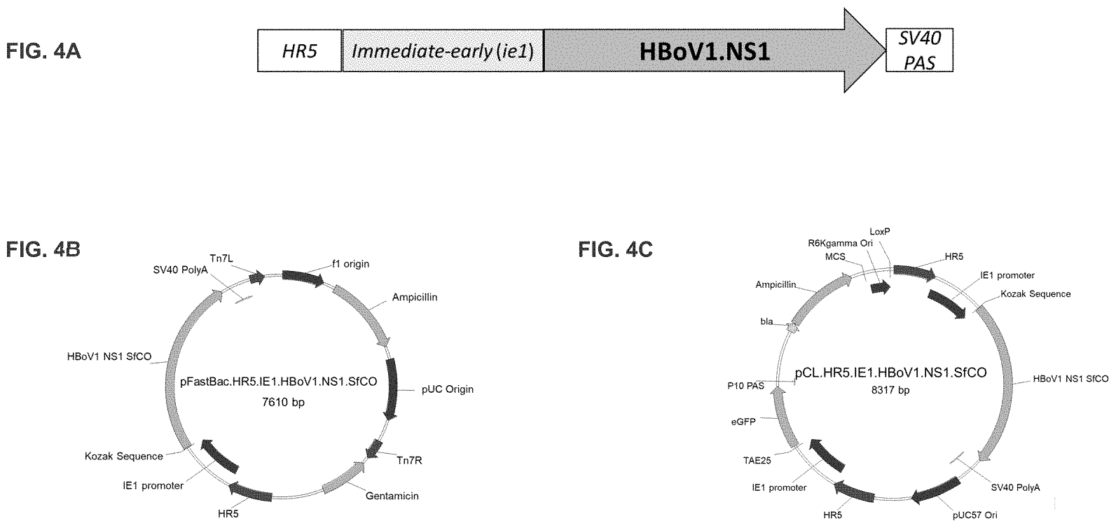

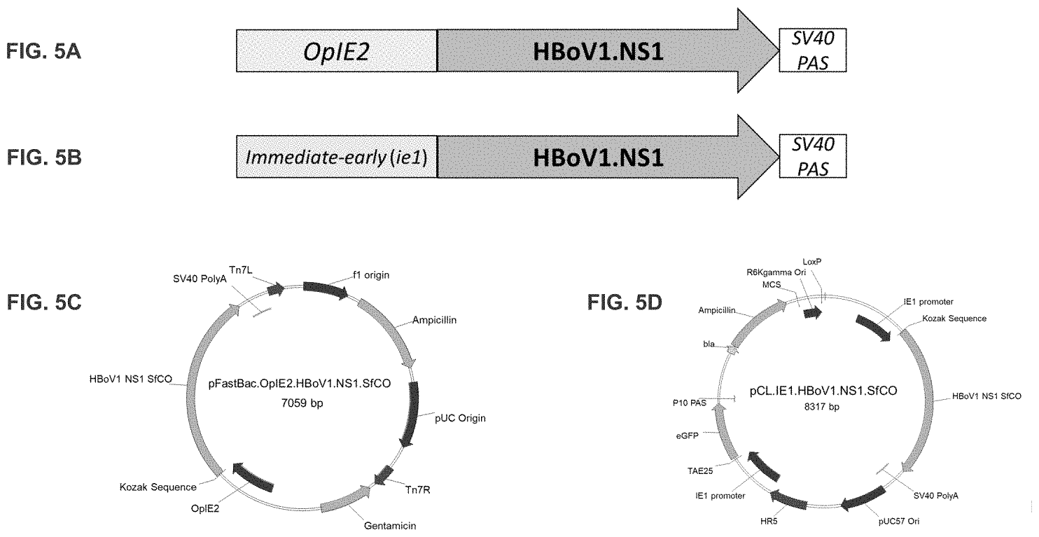

To generate the expression vector, the coding sequence of HBoV1 NS1 was obtained from the HBoV1 genome (GenBank accession no.: JQ923422) and codon-optimized for the Sf cell genome before synthesizing through GenScript® to generate the nucleic acid sequence set forth as SEQ ID NO: 4. The synthetic HBoV1 NS1 DNA was then cloned into the pFastBac1 (Invitrogen) vector under control of the AcMNPV Polyhedrin promoter (FIG. 3A) to generate the pFastBac.Polh.HBoV1.NS1 (FIG. 3B) transfer vector. The synthetic HBoV1 NS1 DNA was also cloned into the pFastBac1 (Invitrogen) vector under control of the immediate-early1 (IE1) promoter preceded by the AcMNPV transcriptional factor hr5 element (FIG. 4A) to generate the pFastBac.HR5.IE1.HBoV1.NS1 (FIG. 4B) transfer vector. The synthetic HBoV1 NS1 DNA was also cloned into pFastBac1 (Invitrogen) vector under the OpMNPV immediate-early2 (IE2) promoter (FIG. 5A) to generate the pFastBac.OpIE2.HBoV1.NS1 (FIG. 5C) transfer vector. These vectors were then transformed into BIVVBac^{DH10B} E. coli to produce recombinant BEVs: AcBIVVBac.Polh.HBoV1.NS1^{Tn7}, AcBIVVBac.HR5.IE1.HBoV1.NS1^{Tn7}, or AcBIVVBac.OpIE2.HBoV1.NS1^{Tn7}, respectively.

These recombinant BEVs were then used for co-infection (TwoBAC) along with FVIIIXTEN BEV in Sf9 cells to generate FVIIIXTEN ceDNA vectors.

HBoV1 NS1 Cre-LoxP Donor Vectors

To investigate the use of a OneBac approach for production of ceDNA, the HBoV1 NS1 gene was inserted at the LoxP site in the recombinant BIVVBac encoding FVIIIXTEN expression cassette at the Tn7 site in the polyhedrin locus. The rationale for inserting both these genes at these sites was to avoid the interference of inverted terminal repeat sequence (ITRs) flanking FVIIIXTEN with the LoxP sequence which is also a palindromic repeat.

In addition, to address the challenges associated with OneBAC system as described above, different promoters of baculovirus genes that are expressed at different time and level during infection cycle were tested to control the levels of HBoV1 NS1 expression in OneBACs encoding both FVIIIXTEN HBoV1 ITRs and NS1.

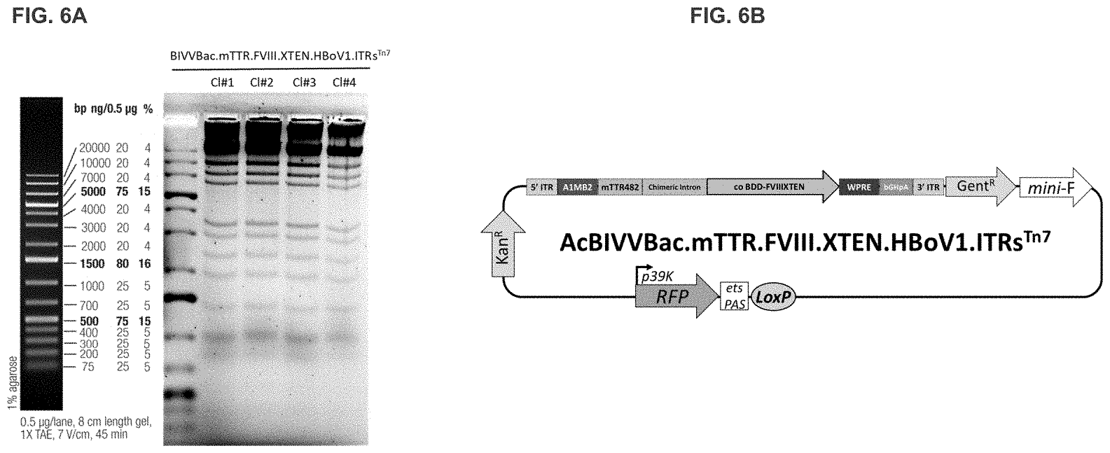

The synthetic Sf-codon-optimized HBoV1 NS1 DNA was cloned into the Cre-LoxP donor vector (as described in U.S. Patent Application No. 63/069,073) under control of the AcMNPV polyhedrin promoter (FIG. 3A) or immediate-early1 promoter preceded with (FIG. 4A) and without (FIG. 5B) the AcMNPV transcriptional enhancer hr5 element to generate pCL.Polh.HBoV1.NS1 (FIG. 3C), pCL.HR5.IE1.HBoV1.NS1 (FIG. 4C), and pCL.IE1.HBoV1.NS1 (FIG. 5D) Cre-LoxP donor vectors, respectively. These constructs were designated with prefix “pCL” for “Plasmid Cre-LoxP”. (see FIGS. 3C, 4C, 5D). The resulting Cre-LoxP donor vectors were then inserted at the LoxP site into the BIVVBac bacmid encoding FVIIIXTEN HBoV1 ITRs at the Tn7 site (FIG. 6B), as described below.

### Example 4: FVIIIXTEN HBoV1 ITRs Baculovirus Expression Vectors (BEVs)

In order to generate recombinant BEV encoding the FVIIIXTEN expression cassette (FIG. 2A) with HBoV1 ITRs, first, the BIVVBac^{DH10B} E. coli (described in, U.S. Patent Application No. 63/069,073) was super-transformed with Tn7 transfer vector, pFastBac.mTTR.FVIIIXTEN.HBoV1.ITRs (FIG. 2B). The transformants were selected on kanamycin, gentamycin, X-Gal, and IPTG. The site-specific transposition of the FVIIIXTEN expression cassette and gentamycin resistance gene at the mini-attTn7 insertion site in BIVVBac disrupted LacZα (fused in-frame with mini-attTn7) and resulted in white E. coli colonies on X-Gal-mediated dual antibiotic selection. Recombinant bacmid DNAs were isolated from white E. coli colonies by alkaline lysis miniprep and digested with restriction enzymes to determine the correct genetic structures. The results of restriction enzyme mapping showed expected fragments for each recombinant bacmid suggesting the site-specific transposition of FVIIIXTEN expression cassette with HBoV1 ITRs in the Polyhedrin locus of BIVVBac (FIG. 6A). Further confirmation was obtained by PCR amplifying regions spanning across the expected insertion site using primers internal and external to the transfer plasmid and sequencing the resulting amplimers (data not shown).

The correct recombinant bacmid encoding FVIIIXTEN expression cassette with HBoV1 ITRs was maxi prep purified and transfected in Sf9 cells using Cellfectin® (Invitrogen) transfection reagent, according to the manufacturer's instructions. At 4-5 days post-transfection, the progeny baculovirus was harvested and plaque purified in Sf9 cells, as previously described. Jarvis et al. (2014), Methods Enzymol., 536: 149-163. Six plaque purified RFP+ clones of recombinant BEV, AcBIVVBac.mTTR.FVIIIXTEN. HBoV1.ITRs^{Tn7} (FIG. 6B) was amplified to P1 (Passage 1) in Sf9 cells seeded at 0.5×10⁶ per mL in T25 flasks in ESF-921 medium supplemented with 10% heat-inactivated Fetal Bovine Serum (FBS). At 4-5 days post-infection, all clones showed progression of infection, determined by the number of RFP+ cells, for each clonal BEV suggesting the virus was able to replicate normally and the insertion of FVIIIXTEN transgene with HBoV1 ITRs in the baculovirus genome had no adverse effect on the progeny virus production. The highest RFP+ clone was selected for further amplification in Sf9 cells to produce working BEV stocks (P2) and then used for co-infection with HBoV1 NS1 BEV in TwoBAC system for ceDNA vector production.

### Example 5: FVIIIXTEN HBoV1 ITRs+HBoV1 NS1 Baculovirus Expression Vectors (BEVs)

In order to test whether BIVVBac could be used to accommodate multiple transgenes, a family of derivative vectors were generated encoding two transgene expression cassettes: 1) FVIIIXTEN HBoV1 ITRs and 2) HBoV1 NS1 under control of different promoters. These BEVs were produced in two steps. First, the FVIIIXTEN expression cassette with HBoV1 ITRs was inserted at the mini-attTn7 site in the Polyhedrin locus in BIVVBac via Tn7 transposition, as described above. Then, the resulting bacmid, BIVVBac.mTTR.FVIIIXTEN.HBoV1.ITRs (FIG. 6B) was used for inserting HBoV1 NS1 expression cassette at the LoxP site in the EGT locus via in-vitro Cre-LoxP recombination using Cre recombinase (New England Biolabs).

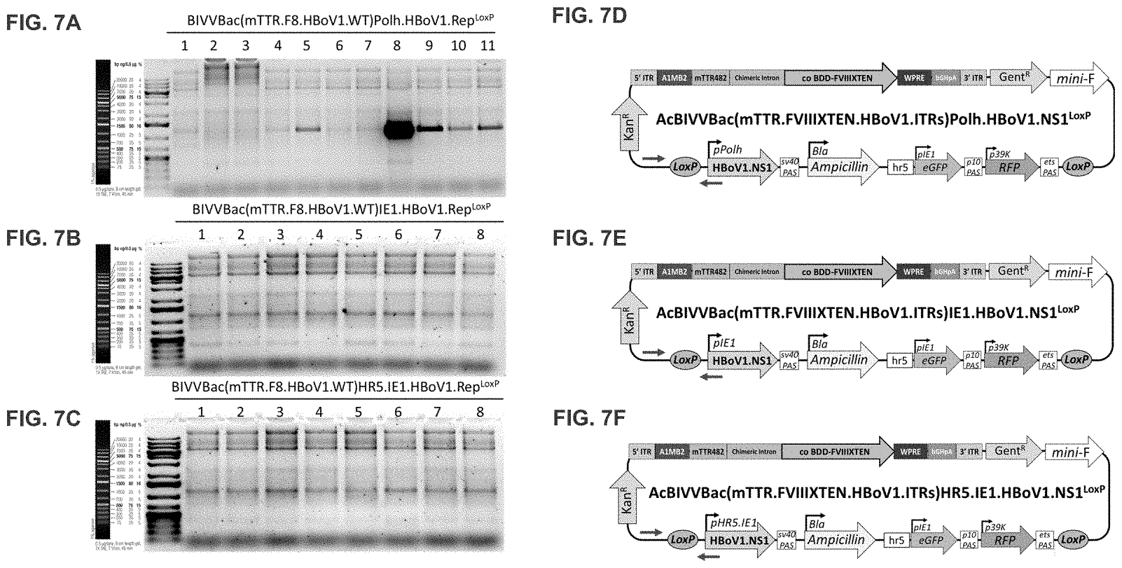

In the process, the Cre-LoxP donor vectors encoding HBoV1 NS1 under the AcMNPV polyhedrin promoter (FIG. 3C) or immediate-early1 (IE1) promoter preceded with (FIG. 4C) and without (FIG. 50) the AcMNPV transcriptional enhancer hr5 element were inserted into the BIVVBac.mTTR.FVIIIXTEN.HBoV1.ITRs (FIG. 6B) bacmid. The recombination reactions were transformed in DH10B E. coli and the transformants were selected on kanamycin, gentamycin, and ampicillin. Triple antibiotic-resistant colonies were screened by the restriction enzyme mapping and/or PCR (FIGS. 7A, 7B, and 7C) by amplifying the regions spanning across the expected insertion site using primers internal and external to the transfer plasmid and sequencing the resulting amplimers.

The correct recombinant bacmid encoding both transgene cassettes was maxi prep purified and transfected in Sf9 cells using Cellfectin® (Invitrogen) transfection reagent. At 4-5 days post-transfection, the progeny baculovirus was harvested and plaque purified in Sf9 cells. Six plaque purified RFP+ and GFP+ clones of each recombinant BEV (AcBIVVBac(mTTR.FVIIIXTEN.HBoV1.ITRs)Polh.HBoV1. NS1^{LoxP}: FIG. 70; AcBIVVBac(mTTR.FVIIIXTEN.HBoV1.ITRs)IE1.HBoV1. NS1^{LoxP}: FIG. 7E; and AcBIVVBac(mTTR.FVIIIXTEN.HBoV1.ITRs)HR5.IE1.HBoV1.NS1^{LoxP}: FIG. 7F) were amplified to P1 (Passage 1) in Sf9 cells seeded at 0.5×10⁶ per mL in T25 flasks in ESF921 medium supplemented with 10% heat-inactivated Fetal Bovine Serum (FBS). At 4-5 days post-infection, all clones showed progression of infection, determined by the number of GFP+ and RFP+ cells, for each recombinant BEV suggesting the virus was able to replicate normally and the insertion of multiple transgenes in the same baculovirus genome had no adverse effect on the progeny virus production. The P1 virus was harvested by low-speed centrifugation and the infected cell pellets were processed either for HBoV1 NS1 detection by immunoblotting. Finally, the highest HBoV1 NS1 expressing clone of each BEV was further amplified to produce working BEV stocks (P2) followed by titering in Sf9 cells. Titrated BEVs were used for infection in Sf9 cells to produce FVIIIXTEN ceDNA vectors, as described below.

### Example 6: FVIIIXTEN HBoV1 ITRs ceDNA Vector Production from OneBAC

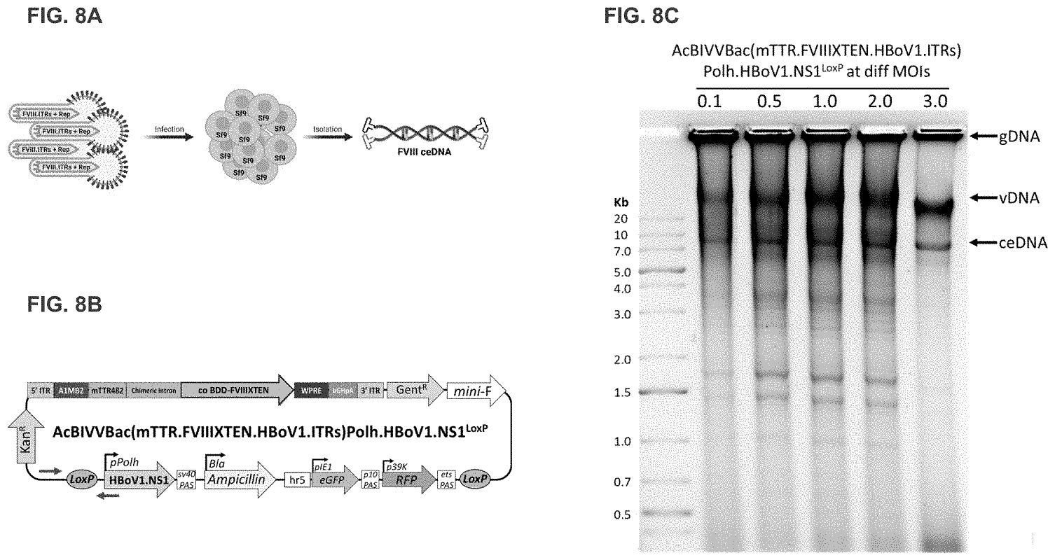

The OneBAC BEVs encoding both FVIIIXTEN HBoV1 ITRs and HBoV1 NS1 genes (FIG. 7D-7F) were tested for FVIIIXTEN ceDNA production in Sf9 cells. About 2.5×10⁶/mL cells were infected with the titrated working stock (P2) of each BEV at multiplicity of infections (MOIs) of 0.1, 0.5, 1.0, 2.0, or 3.0 plaque forming units (pfu)/cell (FIG. 8A). Cells were suspended into 50 mL of serum-free ESF-921 medium and then incubated for 72-96 h or until the cell viability reached at 60-70% in 28° C. shaker incubator. At ˜96 h post-infection, infected cells were harvested, and the pellets were processed for FVIIIXTEN ceDNA vector isolation by PureLink Maxi Prep DNA isolation kit (Invitrogen), according to the manufacturer's instructions. Final elution fractions were analyzed on 0.8 to 1.2% agarose gel electrophoresis to determine the productivity of FVIIIXTEN ceDNA vector.

Agarose gel analysis of the AcBIVVBac (mTTR.FVIIIXTEN.HBoV1.ITRs)Polh.HBoV1.NS1^{LoxP} BEV (FIG. 8B) encoding FVIIIXTEN with HBoV1 ITRs and polyhedrin-driven HBoV1-NS1 is shown in FIG. 8C. The results showed DNA band corresponds to the size of FVIIIXTEN HBoV1 ITRs (˜8.5 kb) ceDNA in all the doses tested with increasing productivity as the MOI increases.

This result was contrary to the ceDNA productivity obtained with AAV2 ITRs OneBAC, where reduction in productivity was previously observed with increases in viral load. Without being bound by theory, the HBoV1-NS1 protein may have a unique mechanism of binding and endonuclease activity at the terminal resolution site of HBoV1 ITRs for DNA replication which could be due to the distinct structures of REH and LEH ITRs (FIG. 2A).

In conclusion, these experiments have shown that the OneBAC approach does prove the concept of ceDNA production from a single recombinant BEV encoding FVIIIXTEN with HBoV1 ITRs and NS1 transgenes. It also shows the feasibility and functionality of multiple transgenes inserted at different loci in a baculovirus shuttle vector (BIVVBac), and its potential use for recombinant AAV vector production in the baculovirus insect cell system.

### Example 7: HBoV1 NS1 (Nonstructural) Baculovirus Expression Vectors (BEVs)

The only structurally characterized parvovirus NS1 N-terminal nuclease domain is from AAV2 Rep, which binds to the consecutive tetranucleotide repeats in the origin of replication (Ori). However, such tetranucleotide repeats are specific to AAV and are not present in HBoV1. Indeed, the LEH (3′ ITR) of the HBoV1 genome forms a loop with a three-way junction, whereas the REH (5′ ITR) is a hairpin with perfect base pairing (FIG. 2A), which are conserved in bocaviruses but distinct from terminal regions of the AAV and parovirus B19 (B19V) genomes. These findings suggest that the mode of NS1 recognition of the Ori in HBoV1 may be distinct from that in AAV. Moreover, AAV is not known to cause human disease and is a dependovirus because virus replication requires a helper virus such as herpesvirus or adenovirus. The HBoV1 NS1 shares as little as 14% sequence identity with AAV Rep. It has been demonstrated that the HBoV1-NS1 contains a positively charged surface that is the putative binding site for the Ori and directly supports the HBoV DNA replication, as in the common rolling-hairpin mechanism proposed for parvoviruses.

HBoV1-NS1 appears to be essential for the ITR-mediated vector production in eukaryotic cells. To investigate the potential ‘rescue’ of an ITR-flanked FVIIIXTEN vector genome from Sf9 cells or FVIIIXTEN BEV, recombinant BEVs encoding HBoV1-NS1 were generated under different baculoviral promoters to optimize the levels of NS1 expression in Sf9 cells.

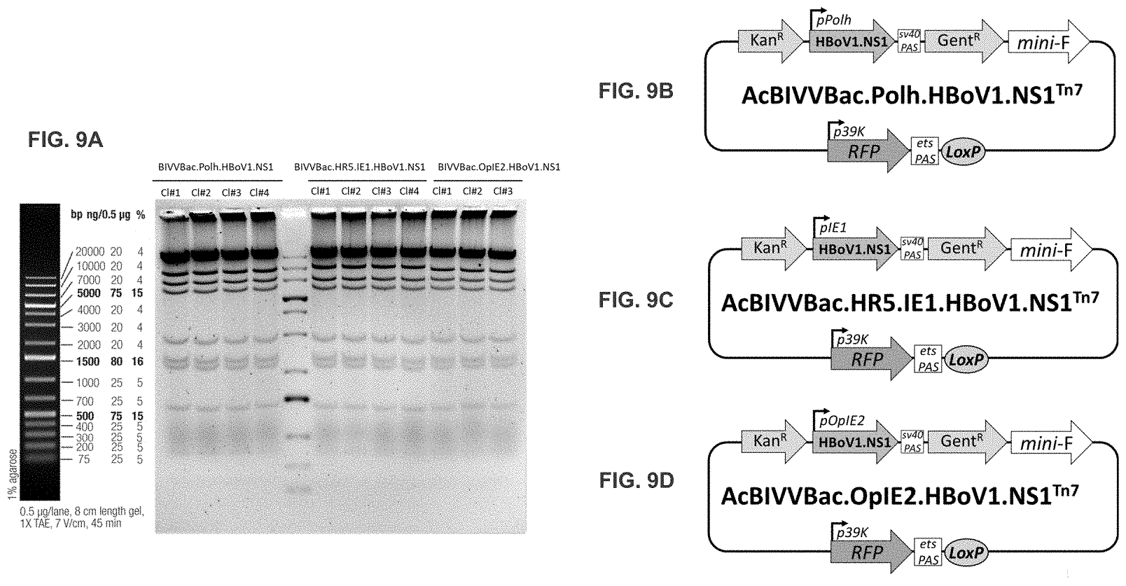

In order to generate these BEVs, BIVVBac^{DH10B} E. coli (see U.S. Patent Application No. 63/069,073) were super-transformed with Tn7 transfer vectors, pFastBac.Polh.HBoV1-NS1 (FIG. 3B), pFastBac.HR5.IE1.HBoV1-NS1 (FIG. 4B), or pFastBac.OpIE2.HBoV1.NS1 (FIG. 5C). Transformants were selected on kanamycin, gentamycin, X-Gal, and IPTG. The site-specific transposition of the HBoV1-NS1 expression cassette and gentamycin resistance gene at the mini-attTn7 insertion site in BIVVBac disrupted LacZα (fused in-frame with mini-attTn7) and resulted in white E. coli colonies on X-Gal-mediated dual antibiotic selection. Thus, recombinant bacmid DNAs were isolated from white E. coli colonies by alkaline lysis miniprep and digested with restriction enzymes to determine the correct genetic structures. The results of restriction enzyme mapping showed expected fragments for each recombinant bacmid suggesting the site-specific transposition of HBoV1-NS1 in the Polyhedrin locus of BIVVBac (FIG. 9A). Further confirmation was obtained by PCR amplifying regions spanning across the expected insertion site using primers internal and external to the transfer plasmid and sequencing the resulting amplimers (data not shown).

The confirmed correct recombinant bacmids encoding Polh.HBoV1-NS1, HR5.IE1.HBoV1-NS1, or OpIE2-HBoV1-NS1 were transfected in Sf9 cells using Cellfectin® (Invitrogen) transfection reagent, according to the manufacturer's instructions. At 4-5 days post-transfection, the progeny baculovirus was harvested and plaque purified in Sf9 cells, as previously described. Jarvis et al. (2014), Methods Enzymol., 536: 149-163. Six plaque purified RFP+ clones of each recombinant BEV, AcBIVVBac.Polh.HBoV1-NS1^{Tn7} (FIG. 9B), AcBIVVBac.HR5.IE1.HBoV1.NS1^{Tn7} (FIG. 9C), and AcBIVVBac.OpIE2.HBoV1.NS1^{Tn7} (FIG. 9D) were amplified to P1 (Passage 1) in Sf9 cells seeded at 0.5×10⁶ per mL in T25 flasks in ESF-921 medium supplemented with 10% heat-inactivated Fetal Bovine Serum (FBS). At 4-5 days post-infection, all clones showed progression of infection, determined by the number of RFP+ cells, for each clonal BEV suggesting the virus was able to replicate normally and the insertion of HBoV1-NS1 in the baculovirus genome had no adverse effect on the progeny virus production.

The highest RFP+ clone was selected for further amplification in Sf9 cells to produce working BEV stocks (P2). The titred virus stock was then used for co-infection with FVIIIXTEN BEV in the TwoBAC system or in the FVIIIXTEN HBoV1 ITRs stable cell line for ceDNA vector production.

### Example 8: FVIIIXTEN HBoV1 ITRs ceDNA Vector Production from TwoBAC

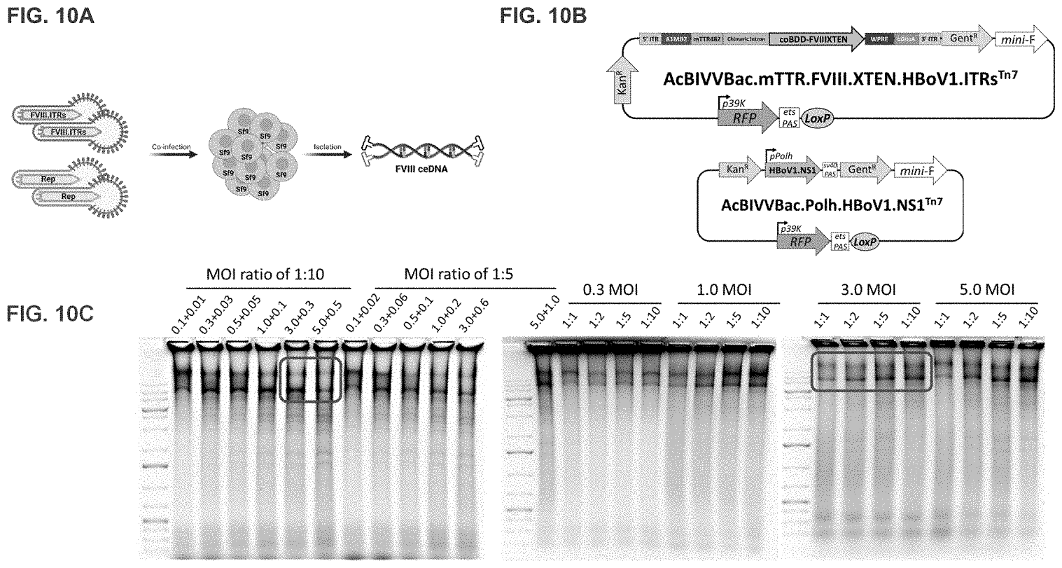

To investigate the TwoBAC approach to transgene expression, clonal recombinant BEV encoding FVIIIXTEN HBoV1 ITRs with polyhedrin-driven HBoV1-NS1 BEV were tested for co-infections at different MOIs of 1:10 and 1:5 ratios or at different ratios of an MOI of 0.3, 1.0, 3.0, and 5.0 pfu/cell for FVIIIXTEN ceDNA vector production in Sf9 cells (FIG. 10A). Specifically, ˜2.0×10⁶/mL cells were seeded in 50 mL of serum-free ESF-921 medium and co-infected with titrated working stocks (P2) of AcBIVVBac.mTTR.FVIIIXTEN.HBoV1.ITRs^{Tn7} BEV at an MOI of 0.1, 0.3, 0.5, 1.0, 3.0, 5.0 pfu/cell with AcBIVVBac.Polh.HBoV1-NS1^{Tn7} BEV at an MOI of 0.01, 0.03, 0.05, 0.1, 0.3, 0.5 pfu/cell for keeping the constant 1:10 ratio or at an MOI of 0.02, 0.06, 0.1, 0.2, 0.6, 1.0 pfu/cell for keeping the constant 1:5 ratio, respectively. Similarly, cells were also co-infected at 1:1, 1:2, 1:5, or 1:10 ratio at a constant MOI of 0.3, 1.0, 3.0, or 5.0 pfu/cell (FIG. 10B). In each case, virus inoculum was not removed, and the cells were incubated until the viability reached at 60-70% in 28° C. shaker incubator. At ˜96 h post-infection, infected cells were harvested, and the pellets were processed for FVIIIXTEN ceDNA vector isolation by PureLink Maxi Prep DNA isolation kit (Invitrogen), according to the manufacturer's instructions. Final elution fractions were analyzed on 0.8 to 1.2% agarose gel electrophoresis to determine the ceDNA productivity.

As expected, agarose gel analyses showed varying degree of FVIIIXTEN ceDNA productivity with different conditions. However, TwoBAC co-infected at an MOI of 3.0 pfu/cell showed increasing levels of FVIIIXTEN ceDNA productivity with increases in ratios of virus load with 1:10 being the highest in comparison to the other conditions tested (FIG. 10C). The higher virus load appears to improve the productivity of FVIIIXTEN HBoV1 ITRs ceDNA, which is consistent with the observation in the OneBAC BEVs (see Example 6). This further suggests the requirement of higher level of HBoV1-NS1 for HBoV1-ITR-dependent FVIIIXTEN ceDNA replication in Sf9 cells.

The results with OneBAC or TwoBAC indicate that the level of HBoV1-NS1 replication has a significant impact on the FVIIIXTEN ceDNA productivity in the baculovirus system.

As an alternative to testing several different conditions of co-infections as discussed above, other ways of improving the productivity of FVIIIXTEN ceDNA were explored by leveraging different promoters of the baculovirus genome. Baculovirus gene promoters are divided into immediate early, early, late, and very late promoters according to their onset of transcription in the infection cycle. Among these, as name indicates, immediate-early (ie) gene promoters are turned-on immediately after the viral infection and remains active throughout the infection cycle. However, the late or very late gene promoters, such as polyhedrin are remained silent until the virus reached to the late stage of infection.

To take advantage of this wide range of promoter selection from the baculovirus genome, the immediate-early1 (IE1) promoter was tested for the HBoV1-NS1. The transcriptional enhancer hr5 element, which has been shown to increase expression levels in Sf9 cells, was included preceding the IE1 promoter. This generated a recombinant BEV encoding HBoV1-NS1 under the control of the AcMNPV immediate-early1 (IE1) promoter preceded by the AcMNPV transcriptional enhancer hr5 element, as depicted in FIG. 9C.

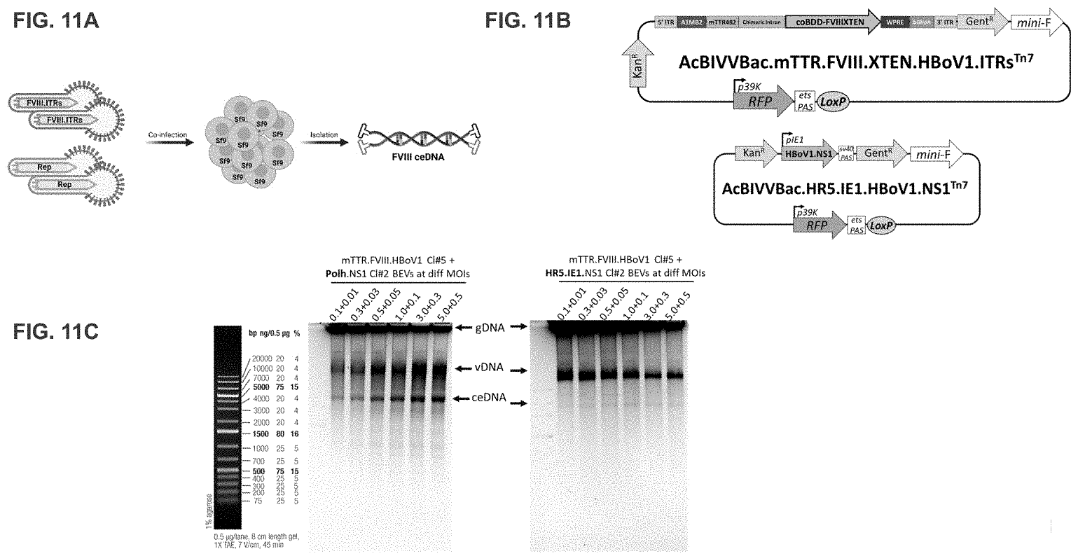

Sf9 cells were co-infected with BEVs encoding FVIIIXTEN HBoV1 ITRs and hr5.IE1-driven HBoV1-NS1 at different MOIs by keeping the constant ratio 1:10, based on the results obtained in FIG. 10C. As positive control, polyhedrin-driven HBoV1-NS1 BEV was included and tested again in the same set of experiments. More specifically, ˜2.0×10⁶/mL Sf9 cells were co-infected with titrated working stocks (P2) of AcBIVVBac.mTTR.FVIIIXTEN.HBoV1.ITRs^{Tn7} BEV (FIG. 10B) at an MOI of 0.1, 0.3, 0.5, 1.0, 3.0, 5.0 pfu/cell with AcBIVVBac.Polh.HBoV1-NS1^{Tn7} OR AcBIVVBac.hr5.IE1.HBoV1-NS1^{Tn7} BEV at an MOI of 0.01, 0.03, 0.05, 0.1, 0.3, 0.5 pfu/cell for keeping the constant 1:10 ratio (FIG. 11B). The remaining procedure followed as described above (see Example 6).

Final elution fractions were analyzed on 0.8 to 1.2% agarose gel electrophoresis to determine the ceDNA productivity. The polyhedrin-driven HBoV1-NS1 co-infection showed increasing levels of FVIIIXTEN ceDNA productivity with increases in MOIs, which further confirms the reproducibility of TwoBAC approach for ceDNA production. However, surprisingly, hr5.IE1-driven HBoV1-NS1 co-infection showed barely detectable levels of FVIIIXTEN ceDNA with no apparent increase in productivity with increases in MOIs, as observed in FIG. 10C.

This data suggests that the early onset of HBoV1-NS1 may not be critical to rescue FVIIIXTEN ceDNA with HBoV1 ITRs. Instead, higher levels of expression later in the infection may be required for efficient rescue and productivity of FVIIIXTEN ceDNA with HBoV1 ITRs. These results further confirm the requirement of higher levels of HBoV1-NS1 for HBoV1-ITR-dependent FVIIIXTEN ceDNA replication in Sf9 cells.

In conclusion, these experiments have shown that the TwoBAC approach does prove the concept of ceDNA production from two recombinant BEVs encoding FVIIIXTEN with HBoV1 ITRs and/or NS1 transgene. These experiments also demonstrate the significance of optimum MOI ratio and/or promoter for achieving higher productivity of FVIIIXTEN ceDNA in Sf9 cells.

### Example 9: FVIIIXTEN HBoV1 ITRs Stable Cell Line

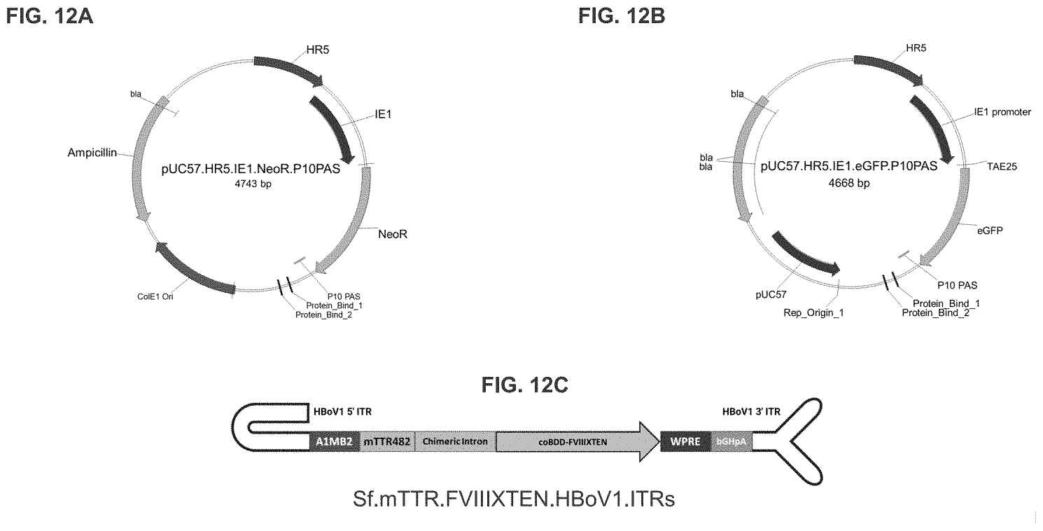

It was hypothesized that the insect cell genome could potentially be modified to produce ceDNA for therapeutic applications following baculovirus infection. To test this hypothesis, plasmids encoding neomycin resistance marker (pUC57.HR5.IE1.NeoR.P10PAS: SEQ ID NO: 7) (FIG. 12A) or enhanced green fluorescent protein (eGFP) (pUC57.HR5.IE1.eGFP.P10PAS: SEQ ID NO: 8) (FIG. 12B) under the control of the AcMNPV immediate early (ie1) promoter preceded by the transcriptional enhancer hr5 element and followed by the AcMNPV p10 polyadenylation signal was synthesized from GenScript® (Piscataway, N.J.)

These plasmids were co-transfected with the plasmid encoding FVIIIXTEN with HBoV1 ITRs (Sf.mTTR.FVIIIXTEN.HBoV1.ITRs) (FIG. 12C) in Sf9 cells using a modified calcium phosphate transfection method. At 24 h post-transfection, cells were visualized under the fluorescence microscope to determine the transfection efficiency and the results showed >80% GFP+ cells suggesting higher transfection efficiency. At 72 h post-transfection, cells were selected with G418 antibiotic (Sigma Aldrich) suspended in complete TNMFH medium (Grace's Insect Medium supplemented with 10% FBS+0.1% Pluronic F68) at 1.0 mg/mL final concentration. After about a week of selection, there were ˜50% of transformed cells recovered which suggests that the neomycin resistant marker was stably integrated into this cell population. The survivor cells were taken off the selection media and fed with a fresh complete TNMFH medium until confluence growth. The confluent cells were progressively expanded as an adherent culture into larger culture vessels as they continue to divide. Later, polyclonal cell population was adapted to the suspension culture by growth in shake flasks for one passage in complete TNMFH and one passage in ESF-921 medium supplemented with 10% FBS. Finally, cells were adapted to serum-free ESF-921 in shake flasks as suspension cultures. These shake flask cultures were routinely maintained in serum-free ESF-921 medium with passages every four days and cell growth was monitored.

### Example 10: FVIIIXTEN ceDNA Purification

In the baculovirus-insect cell system, recombinant BEV delivers the gene of interest under a strong promoter and provides transcriptional complex essential for the virus replication in insect cells. Typically, the baculovirus DNA genome replicates in the nucleus and produce several tens of millions of progeny virus particles, each containing a full-length DNA genome. It has been demonstrated that baculoviral genomic DNAs are co-purified with the ceDNA while isolating DNA from the insect cells using a plasmid DNA-based purification method such as silica gel columns. The commercial plasmid DNA kit columns are generally not designed to separate DNA based on their molecular weights and therefore, typically, all forms of DNA present in the sample can bind to these columns. Moreover, the binding capacity of large molecular weight DNA could be different than the low molecular weight DNA and the anion-exchange based kit columns are not optimized based on the binding efficiency of different sizes of DNA.

It was hypothesized that the high molecular weight DNAs (>20 kb) observed in ceDNA preps were most likely baculoviral and/or Sf9 cell genomic DNAs that were co-purified with the low molecular weight FVIIIXTEN ceDNA (˜8.5 kb) (see, e.g., FIGS. 8C, 10C, and 11C).

Previously, an indirect approach was employed to reduce the baculoviral DNA by knocking out a baculoviral capsid gene such as VP80, which is required for the infectious progeny virus production. This approach showed significant reduction in the baculoviral DNA in the ceDNA prep obtained from the knock-out BEV (see U.S. Patent Application No. 63/069,115). Though this approach was efficient in reducing the baculoviral DNA contamination, it was unable to reduce the cellular genomic DNAs, which were present in a significant quantity (˜60%) of the total DNAs obtained from infected cell pellets.

Therefore, a direct approach of separating FVIIIXTEN ceDNA from the rest of the unwanted DNAs was employed and demonstrated to efficiently obtain the purified FVIIIXTEN (>95% purity) from the total DNA prep from the infected cell pellet. This novel approach leverages preparative electrophoresis, which is widely used for separating different protein molecules according to their size and charge. See, e.g., Michov, B. (2020) Electrophoresis. Berlin, Boston: De Gruyter, pp. 405-424. For example, the Bio-Rad Model 491 prep cell or other such units can be used to separate complex molecules based on their sizes.

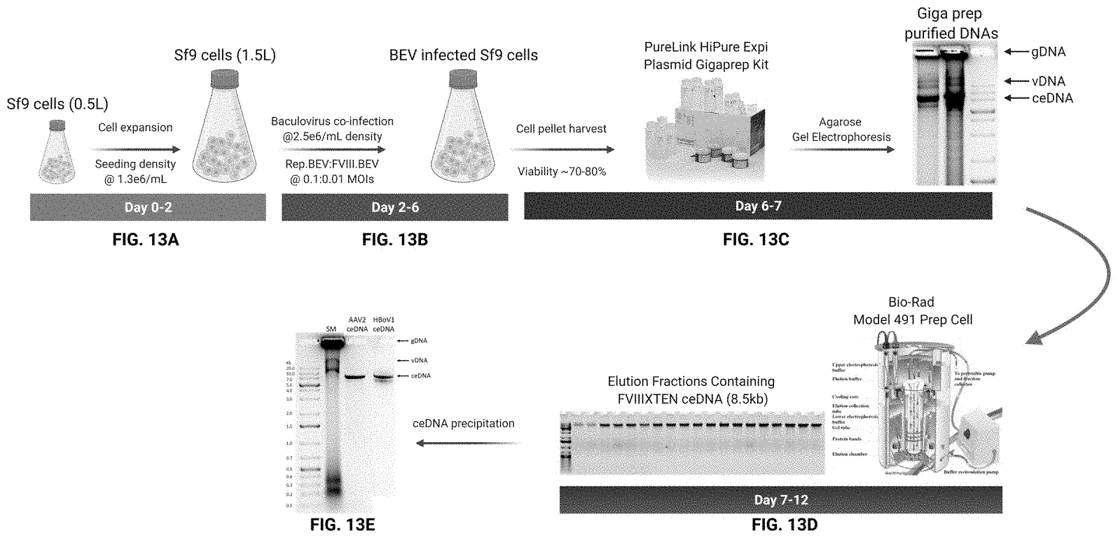

The entire workflow of ceDNA purification is shown in FIG. 13, where the process starts with scaling up the Sf9 cell culture from 0.5 L to 1.5 L or higher volume in serum-free insect cell culture medium (FIG. 13A). Upon reaching the desired cell density of ˜2.5×10⁶/mL, typically after 2 days of incubation with a seeding density of ˜1.3×10⁶/mL, cells are infected with OneBAC or TwoBAC BEVs (depending on the approach used for ceDNA production) at an optimized MOI and let the cells incubated in 28° C. shaker incubator until the viability reached at ˜60-70% which typically takes about 4 days (FIG. 13B). Once the viability reached at ˜70%, cells are harvested and processed for total DNA purification by anion-exchange chromatographic kit columns, such as PureLink HiPure Expi Plasmid Gigaprep purification kit (Invitrogen), according to the manufacturer's instructions. An aliquot of purified DNA material is checked on 0.8 to 1.2% agarose gel electrophoresis to determine the DNA productivity and integrity (FIG. 13C).

The purified material is then loaded onto a Preparative Agarose Gel Electrophoresis Unit, containing a 0.5% preparative agarose gel and a 0.25% stacking agarose gel, assembled according to the manufacturer's instructions. Samples are run at low voltage (˜40 constant volts) at 4° C. for 6-7 days with a buffer recirculation flow rate of ˜50 mL/min and the elution buffer rate of 50 μL/min to collect each fraction at 70-80 min in the fraction collection chamber. After continuous elution electrophoresis, 20 μL of each fraction is checked on 0.8 to 1.2% agarose gel electrophoresis to determine the purity of FVIIIXTEN ceDNA (FIG. 13D). The desired fractions are combined to precipitate with 3M NaOAc, pH 5.5 and 100% EtOH at −20C for 1-2 h. Finally, the precipitated FVIIIXTEN ceDNA is pelleted at high-speed and washed once with 70% EtOH before resuspending into the TE, pH 8.0 buffer. Purified FVIIIXTEN ceDNA is again checked on 0.8 to 1.2% agarose gel electrophoresis to confirm the purity and integrity before injecting into the animals for in vivo efficacy studies (FIG. 13E).

### Example 11: FVIIIXTEN HBoV1 ITRs In Vivo Efficacy

ssFVIIIXTEN HBoV1 ITRs (Single-Stranded DNA)

It was hypothesized that the hairpin formed within the ITR region enables higher levels of long-term persistent transgene expression. To investigate the functionality of HBoV1 ITRs in vivo, single-stranded DNA (ssDNA) comprising codon-optimized human FVIIIXTEN with preformed HBoV1 ITRs was tested in hFVIIIR593C^{+/+}/HemA mice. These mice contain a human FVIII-R593C transgene, designed with the murine albumin (Alb) promoter driving expression of an altered human coagulation factor VIII (FVIII) cDNA harboring a mutation that is frequently observed in patients with mild hemophilia A. These mice also carry a knock-out of the FVIII gene and are deficient for endogenous FVIII protein. These double mutant mice are tolerant of human FVIII injection and have no FVIII activity. They produce very little inhibitory antibodies and lack FVIII responsive T cells or B cells after treatment with human FVIII. The hFVIIIR593C^{+/+}/HemA mouse is further described in Bril, et al. (2006) Thromb. Haemost. 95(2): 341-7.

Single-stranded FVIIIXTEN (ssFVIIIXTEN) with preformed HBoV1 ITRs was generated by denaturing the double-stranded DNA fragment products (FVIII expression cassette and plasmid backbone) of PvuII digestion at 95° C. and then cooling down at 4° C. to allow the palindromic ITR sequences to fold. Then, the ssFVIIIXTEN was systemically injected via hydrodynamic tail-vein injections at 10 μg or 40 μg/mouse, which is equivalent to 400 μg or 1600 μg/kg, respectively. Plasma samples were collected from injected mice at 7 days interval for 5.5 months. Plasma FVIII activity was measured by the Chromogenix Coatest® SP Factor VIII chromogenic assay, according to the manufacturer's instructions.

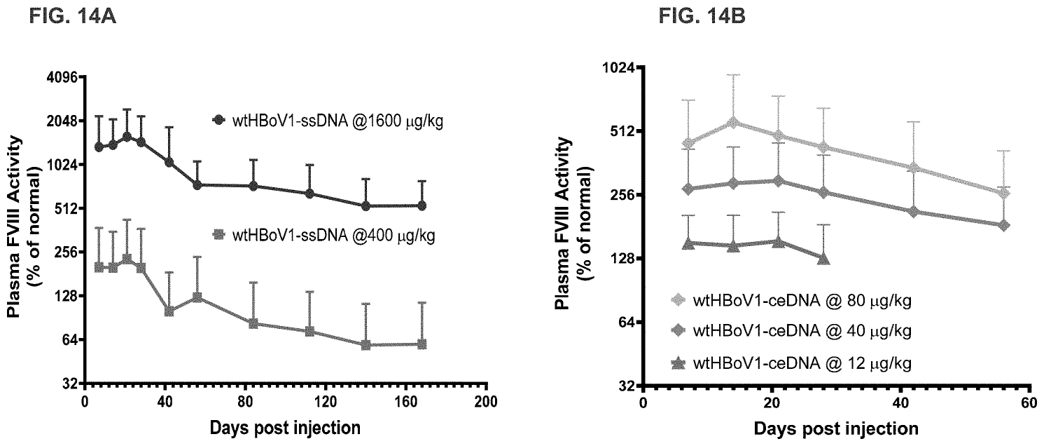

The plasma FVIII activity normalized to percent of normal for ssFVIIIXTEN injected animals is shown in FIG. 14A. The results showed dose-dependent response in HemA mice over the course of 5.5 months with supraphysiological levels (>1000% of normal) of FVIII expression in both the doses tested. However, there was initial drop in FVIII expression observed up to day 56 but then the levels were stabilized up to day 168 suggesting the persistent expression of HBoV1 ITRs flanked ssFVIIIXTEN from the liver of injected animals. Thus, these results validate the functionality of HBoV1 ITRs for long-term persistent expression of FVIIIXTEN in vivo.

ceFVIIIXTEN HBoV1 ITRs (Closed-End DNA)

To validate the functionality of HBoV1 ITRs in ceDNA, ceFVIIIXTEN purified from the infected Sf9 cell pellets, as described above was injected systemically via hydrodynamic tail-vein injections in hFVIIIR593C^{+/+}/HemA mice at 0.3 μg, 1.0 μg, or 2.0 μg/mouse, which is equivalent to 12 μg, 40 μg, and 80 μg/kg, respectively. Plasma samples from injected mice were collected at 7 days interval and FVIII activity was measured by the chromogenic assay, as described above.

The plasma FVIII activity normalized to percent of normal for ceFVIIIXTEN injected animals is shown in FIG. 14B. The results of this study show dose-dependent response in HemA mice with supraphysiological levels (>500% of normal) of FVIII expression observed in the highest dose tested up to day 56 post injection. Interestingly, a similar level of expression was achieved when the mice were injected with ssFVIIIXTEN at 1600 μg/kg, which is at least 20×higher the dose of ceFVIIIXTEN (80 μg/kg) (FIG. 14A-14B). This data suggests that ceDNA provides higher levels of FVIII expression in comparison to the ssDNA form.

In conclusion, these in vivo studies validate the functionality of HBoV1 ITRs either in the ssDNA or ceDNA form and demonstrates that HBoV1 ITRs can be used to produce functional ceDNA encoding a transgene of interest in the baculovirus insect cell system.

### Example 12: Improved ceDNA Vector Purity Using CRISPR Cas Knock Out of VP80 in HBoV1 NS1 BEVs

The HBoV1 NS1 expressed under the AcMNPV polyhedrin promoter indeed was able to rescue the HBoV1 ITR-flanked FVIIIXTEN and proves the concept of ceDNA production with HBoV1 ITRs in the baculovirus system. However, significant levels of baculoviral DNA (vDNA) contamination were observed in the ceDNA preps, presumably due to the higher virus load required in comparison with the AAV2 Rep-BEV to achieve higher ceDNA productivity. The high molecular weight DNA (>20 kb) observed in these ceDNA preps (FIG. 8C, FIG. 10C) were most likely the baculoviral genomic DNA that were co-purified with the low molecular weight ceDNA (˜8 kb).

To reduce the baculoviral DNA contamination in ceDNA preps, an indirect approach of knocking out VP80, an essential gene of the baculovirus genome that is required for producing infectious virus particles in insect cells (Sf9), was implemented. VP80 was knocked out in all three NS1 BEVs (FIGS. 9B, 9C, and 9D) using Alt-R CRISPR-Cas9 system (see U.S. Patent Application No. 63/069,115). This approach potentially reduces the number of progeny virus particles and ultimately the baculoviral DNA contamination in the ceDNA preparations.

CRISPR-Cas9 Knock-Out of AcMNPV VP80 Gene:

The recombinant BEVs encoding HBoV1 NS1 under the AcMNPV polyhedrin (FIG. 9B) or the OpMNPV OpIE2 (FIG. 9C) promoter were selected to knock-out vp80 gene by the CRISPR-Cas9 system, as previously described (see, e.g., International Application No. PCT/US2021/047202).

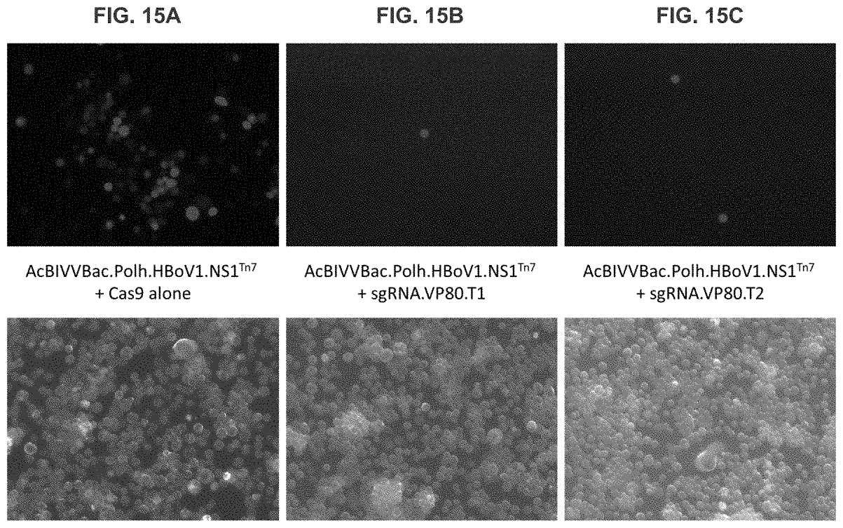

Briefly, two crRNAs targeting the coding sequence were designed and used for generating functional sgRNAs using the Alt-R CRISPR-Cas9 system (Integrated DNA Technology™), according to the manufacturer's instructions. Each sgRNA was then co-transfected with the SpCas9 nuclease and AcBIVVBac.Polh.HBoV1.NS1^{Tn7} or AcBIVVBac.OpIE2.HBoV1.NS1^{Tn7} bacmid DNA in Sf9 cells, seeded at 0.5×106 per mL in T25 flasks in serum-free ESF-921 medium, using Cellfectin® (Invitrogen™) transfection reagent. At 4-5 days post-transfection, cells were visualized under the fluorescence microscope and the results showed ˜10% RFP+ cells for both the sgRNA targets. Exemplary fluorescence microscopic images of infected cells are shown in FIG. 15. The cells infected with AcBIVVBac.Polh.HBoV1.NS1^{Tn7} BEV in Cas9 alone showed progression of infection as expected (FIG. 15A) however, in contrast, the sgRNA.VP80.T1 (FIG. 15B) or sgRNA.VP80.T2 (FIG. 15C) treated cells showed restricted infection to individual cells, which is likely due to the knockout of VP80.

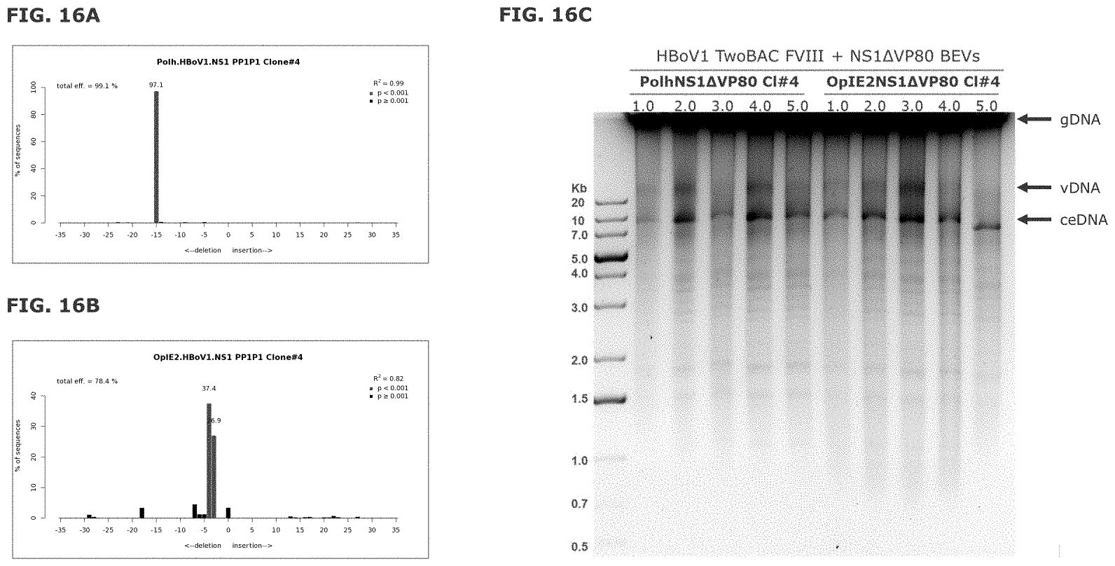

To determine the indels induced by each sgRNA, the progeny baculovirus was harvested and plaque purified in a complement Sf.39K.VP80 cell line, as described previously. Jarvis et al. (2014), Methods Enzymol., 536: 149-163. At 5-6 days post-infection, twelve plaque purified RFP+ clones were amplified to P1 in Sf.39K.VP80 cells seeded at 0.5×10⁶ per mL in T25 flasks in ESF-921 medium supplemented with 10% FBS. The fluorescence microscopic observation of the amplified clones showed −80% RFP+ cells which suggest that the Sf.39K.VP80 cell line was able to complement the VP80 function in trans for progeny virus production. Each clonal BEV was harvested by the low-speed centrifugation and the cell pellet was then used for total DNA isolation by the Qiagen's DNeasy Blood and Tissue genomic DNA isolation kit (catalog no. 69506), according to the manufacturer's instructions. The resulting DNA was used as a template for PCR amplification of each target sequence with primers specific to the AcMNPV vp80 coding sequence. The PCR amplimers were then gel purified and directly sequenced through the Genewiz sequencing facility. The resulting sequences were analyzed by the TIDE (Tracking of Indels by DEcomposition) program (tide.deskgen.com) using default settings to determine the indels induced by each sgRNA. The TIDE analyses showed frameshift mutations in sgRNA.T1 treated AcBIVVBac.Polh.HBoV1.NS1^{Tn7} BEV Clone #4 with the highest (97.1%) −15 bp deletions (FIG. 16A) and AcBIVVBac.OpIE2.HBoV1.NS1^{Tn7} BEV Clone #4 with the highest (37.4%) −4 bp and (26.9%) −3 bp deletions (FIG. 16B) in the vp80 coding sequence with no detectable insertions. Each clone was amplified to P2 to generate working BEV stock followed by titering in Sf.39K.VP80 cells, as described previously. Jarvis et al. (2014), Methods Enzymol., 536: 149-163. Titrated working stock of vp80KO BEVs was then used for co-infection in TwoBAC system for FVIIIXTEN HBoV1 ceDNA vector production.

Human FVIIIXTEN ceDNA Production Using vp80KO BEVs:

About 2.0×10⁶ cells were seeded in 100 mL of serum-free ESF-921 medium and were co-infected with titrated working PP1P2 stocks of AcBIVVBac.FVIIIXTEN.HBoV1.ITRs^{Tn7} and AcBIVVBac.Polh.HBoV1.NS1ΔVP80^{Tn7} or AcMNPV.OpIE2.HBoV1.NS1A VP80^{Tn7} BEVs at an MOI of 1.0, 2.0, 3.0, 4.0, and 5.0 pfu/cell. In each case, virus inoculum was not removed, and the cells were incubated until the viability reached at 60-70% in 28° C. shaker incubator. At −96 h post-infection, infected cells were harvested, and the pellets were processed for FVIIIXTEN HBoV1 ceDNA isolation by PureLink Maxi Prep DNA isolation kit (Invitrogen), according to the manufacturer's instructions.

Final elution fractions were analyzed by 0.8 to 1.2% agarose gel electrophoresis to determine the ceDNA productivity and purity. Agarose gel analyses showed very low to no detectable high molecular weight (>20 kb) baculoviral DNA (vDNA) contamination in vp80KO BEVs expressing HBoV1 NS1 under the AcMNPV polyhedrin or the OpMNPV OpIE2 promoter (FIG. 16C). This suggests that the vp80KO approach was able to reduce the contaminating baculoviral DNA and simultaneously improve the FVIIIXTEN HboV1 ceDNA yield when cells were co-infected at an MOIs of 2.0, 3.0 or 4.0 pfu/cell (FIG. 11C, FIG. 16C).

### Example 13: FVIIIXTEN HBoV1 ceDNA Vector Production from TwoBAC System

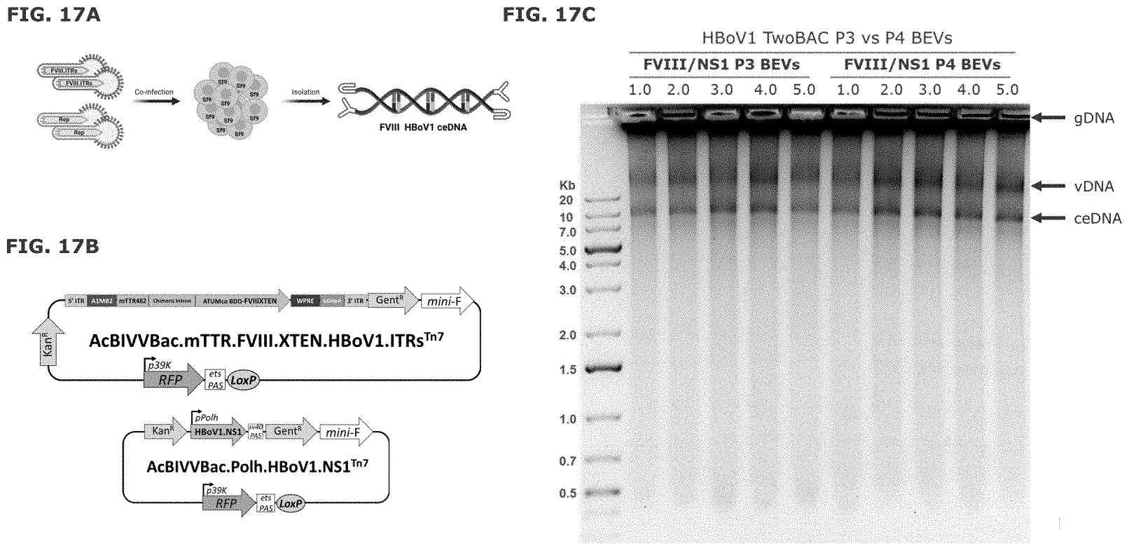

Genetic instability is one of the major concerns in the field of baculovirology and especially over several passages of recombinant baculoviruses in Sf9 cells. In addition, baculovirus genomes contain several homologous regions (hrs) that are prone to recombine over passages in Sf9 cells and can potentially lose the transgene in the recombinant BEVs. Inverted terminal repeats (ITRs) are also palindromic repeat sequences and can potentially recombine at different loci in the baculovirus genome, considering the large size of baculoviral DNA. Therefore, to determine the genetic stability of recombinant BEV encoding a FVIIIXTEN gene under the liver-specific mTTR promoter with HBoV1 WT ITRs, the BEV was sequentially amplified by infecting Sf9 cells at an MOI of 0.1 pfu/cell, as previously described. Jarvis et al. (2014), Methods Enzymol., 536: 149-163. The resulting recombinant BEVs were tested for FVIIIXTEN HBoV1 ceDNA production using the TwoBAC system (see FIG. 17A and construct depicted in FIG. 17B).

About 2.0×10⁶ cells were seeded in 100 mL of serum-free ESF-921 medium and co-infected with titrated working stocks (P3 or P4) of AcBIVVBac.mTTR.FVIIIXTEN. HBoV1.ITRs^{Tn7} and AcBIVVBac.Polh.HBoV1.NS1^{Tn7} BEVs at an MOI of 1.0, 2.0, 3.0, 4.0, and 5.0 pfu/cell. In each case, virus inoculum was not removed, and the cells were incubated until the viability reached at 60-70% in 28 oC shaker incubator. At −96 h post-infection, infected cells were harvested, and the pellets were processed for FVIIIXTEN HBoV1 ceDNA isolation by PureLink Maxi Prep DNA isolation kit (Invitrogen), according to the manufacturer's instructions.

Final elution fractions were analyzed by 0.8 to 1.2% agarose gel electrophoresis to determine the ceDNA productivity. Agarose gel analysis as shown in FIG. 17C showed almost equivalent levels of FVIIIXTEN HBoV1 ceDNA productivity with P3 or P4 (and P5, data not shown) BEVs suggesting recombinant BEVs encoding FVIIIXTEN HBoV1 ITRs are genetically stable up in later passages in Sf9 cells.

### Example 14: FVIIIXTEN HBoV1 ceDNA Vector Production from OneBAC System

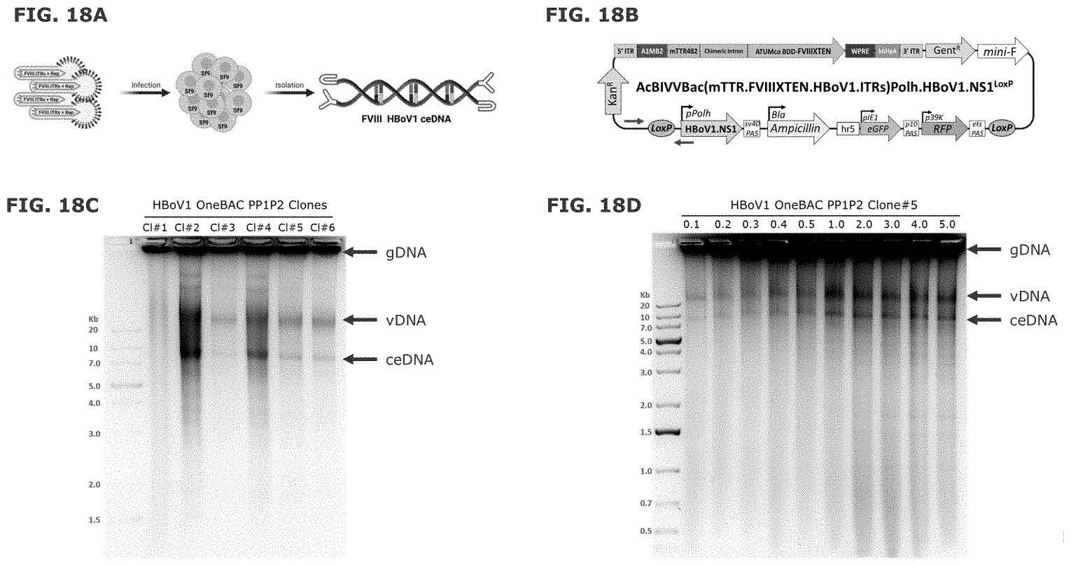

The HBoV1 OneBAC system has been shown to produce FVIIIXTEN HBoV1 ceDNA vector in Sf9 cells (see, e.g., FIG. 8C). However, proof-of-concept was achieved using polyclonal recombinant BEVs. To support larger scale productions, clonal BEVs need to be generated. Accordingly, in this study, HBoV1 OneBAC polyclonal BEVs were plaque-purified and amplified in Sf9 cells (see FIG. 18A). These clonal OneBAC BEVs were then screened for FVIIIXTEN HBoV1 ceDNA vector production in Sf9 cells.

Plaque purification and amplification of recombinant HBoV1 OneBAC BEVs was performed as described previously. Jarvis et al. (2014), Methods Enzymol., 536: 149-163. Six plaque-purified clones were amplified to P2 by infecting ˜1.0×106/mL Sf9 cells in 100 mL of ESF-921 medium supplemented with 10% fetal-bovine serum and incubated for 4-5 days or until the cell viability reached at 60-70% in 28° C. shaker incubator. At 4-5 days post infection, cell-free supernatant was harvested and stored as P2 working stocks and the cell pellets were processed for FVIIIXTEN HBoV1 ceDNA isolation by PureLink Maxi Prep DNA isolation kit (Invitrogen), according to the manufacturer's instructions.

Final elution fractions were analyzed by 0.8 to 1.2% agarose gel electrophoresis to determine the FVIIIXTEN HBoV1 ceDNA vector productivity. FIG. 18C shows agarose gel analysis of HBoV1 OneBAC encoding FVIIIXTEN with HBoV1 ITRs and polyhedrin-driven HBoV1-NS1 (construct depicted in FIG. 18B). The results showed varying degree of HBoV1 ceDNA productivity for different clones with clone #2 and clone #4 being the higher producers of HBoV1 ceDNA in comparison with other clones tested (FIG. 18C). This result shows the variability in different baculoviral clones obtained from the same stock and highlights the importance of using clonal recombinant BEVs for large scale ceDNA manufacturing.

To determine the optimum productivity of clonal HBoV1 OneBAC BEV, about 2.0×10⁶ cells were infected with titrated working stock (P2) of HBoV1 OneBAC BEV clone #5 at an MOI of 0.1, 0.2, 0.3, 0.4, 0.5, 1.0, 2.0, 3.0, 4.0, or 5.0 pfu/cell. In each case, virus inoculum was not removed, and cell were incubated until the cell viability reached 60-70% in a 28° C. shaker incubator. At ˜96 h post-infection, infected cells were harvested, and the pellets were processed for FVIIIXTEN HBoV1 ceDNA isolation by PureLink Maxi Prep DNA isolation kit (Invitrogen), according to the manufacturer's instructions. Final elution fractions were analyzed by 0.8 to 1.2% agarose gel electrophoresis to determine the FVIIIXTEN HBoV1 ceDNA productivity.

Agarose gel analysis showed DNA band corresponds to the size of FVIIIXTEN HBoV1 ceDNA (˜8.5 kb) in all doses tested with increasing productivity as the MOI increases. This result was contrary to the ceDNA productivity obtained with AAV2 ITRs OneBAC, where reduction in productivity with increases in viral load was observed. This HBoV1 OneBAC approach proves the concept of ceDNA production from a single recombinant BEV encoding FVIIIXTEN with HBoV1 ITRs and NS1 transgenes. It also shows the feasibility and functionality of multiple transgenes inserted at different loci in a baculovirus shuttle vector (BIVVBac).

### Example 15: FVIIIXTEN HBoV1 ssDNA vs ceDNA In Vivo Efficacy

ssFVIIIXTEN HBoV1 ITRs (Single-Stranded DNA)

It was hypothesized that the hairpin formed within the HBoV1 ITR region drives the long-term persistent expression of transgene at higher levels. To validate the functionality of HBoV1 ITRs in vivo, single-stranded DNA (ssDNA) comprising codon-optimized human FVIIIXTEN (ssFVIIIXTEN) with preformed HBoV1 ITRs was tested in hFVIIIR593C^{+/+}/HemA mice.

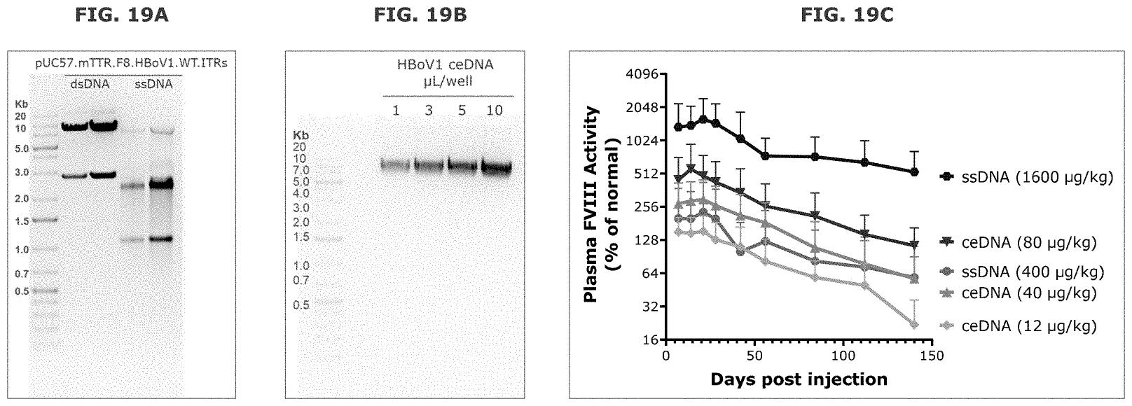

The ssFVIIIXTEN with preformed HBoV1 ITRs was generated by denaturing the double-stranded DNA (dsDNA) fragment products (FVIII expression cassette and plasmid backbone) of PmII digestion at 95° C. and then cooling down at 4° C. to allow the palindromic ITR sequences to fold. The resulting ssFVIIIXTEN was confirmed by 0.8 to 1.2% agarose gel electrophoresis. The gel analysis showed half the size of dsDNA for ssFVIIIXTEN suggesting efficient hairpin formation (FIG. 19A). The ssFVIIIXTEN was systemically injected via hydrodynamic tail-vein injections at either 10 μg or 40 μg/mouse, which is equivalent to 400 μg or 1600 μg/kg, respectively. Plasma samples were collected from injected mice at 7 day intervals for 5.5 months. Plasma FVIII activity was measured by the Chromogenix Coatest® SP Factor VIII chromogenic assay, according to the manufacturer's instructions.

The plasma FVIII activity normalized to percent of normal for ssFVIIIXTEN injected animals is shown in FIG. 19C. The results showed dose-dependent response in HemA mice over the course of 5.5 months with supraphysiological levels (>1000% of normal) of FVIII expression in higher dose cohorts. However, there was an initial drop in FVIII expression observed up to day 56 and the levels were stabilized up to day 140, suggesting persistent expression of HBoV1 ITRs flanked ssFVIIIXTEN from the liver. These results validate the functionality of HBoV1 ITRs for long-term persistent expression of FVIIIXTEN in vivo.

ceFVIIIXTEN HBoV1 ITRs (Closed-End DNA)

There is a major structural difference between closed-end DNA (ceDNA) and single-stranded DNA (ssDNA), where the former is a double-stranded and later is the single-stranded, respectively. This difference may impact levels of expression as well as the stability of the nucleic acid molecule. This study shows the functionality of HBoV1 ITRs in ceDNA form in vivo. To test this, ceFVIIIXTEN was obtained using the TwoBac approach as described in Example 8, was purified from infected Sf9 cell pellets, and the quality was determined by 0.8 to 1.2% agarose gel electrophoresis. Agarose gel analysis showed >90% purity of ceFVIIIXTEN with no detectable contaminating DNAs (FIG. 19B).

The resulting ceFVIIIXTEN was injected systemically via hydrodynamic tail-vein injections in hFVIIIR593C^{+/+}/HemA mice at 0.3 μg, 1.0 μg, or 2.0 μg/mouse, which is equivalent to 12 μg, 40 μg, and 80 μg/kg, respectively. Plasma samples from injected mice were collected at 7 day intervals and FVIII activity was measured by the chromogenic assay, as described above. The plasma FVIII activity normalized to percent of normal for ceFVIIIXTEN injected animals is shown in FIG. 19C.

The results showed dose-dependent response in HemA mice with supraphysiological levels (>500% of normal) of FVIII expression at the highest dose (80 μg/kg) tested for ceFVIIIXTEN. The highest levels of FVIII expression for ceDNA was 2×lower than the highest levels achieved by ssFVIIIXTEN at 1600 μg/kg. However, ssDNA was dosed at much higher amounts to achieve these high levels of FVIII expression. ceDNA appears to provide higher levels of FVIII expression per dosage amount. For example, the FVIII expression levels for ssDNA at 400 μg/kg and ceDNA at 40 μg/kg were comparable (FIG. 19C). These in vivo studies validate the functionality of HBoV1 ITRs either in the ssDNA or ceDNA form and show that HBoV1 ITRs can be used to produce functional ceDNA encoding transgene of interest in the baculovirus insect cell system.

### Example 16: FVIIIXTEN HBoV1 Monomeric Vs Multimeric ceDNA In Vivo Efficacy

Recombinant AAV genomes have been shown to persist episomally and their episomal existence is thought to be correlated with long-term transgene expression. These genomes appeared to originate through a monomeric circularization process, leading to head-to-tail AAV circular genome. However, over time, there is a decline in monomer circular intermediates in favor of high-molecular-weight circular concatamers. Additional details are disclosed in Duan et al. (1998), J Virol. 72(11); 8568-8577. Presently little is known about episomal existence of closed-end DNA (ceDNA) and the benefits of monomeric over concatameric forms of ceDNA in vivo.

This study was performed to determine the impact of monomeric versus multimeric forms of ceDNA in vivo by testing both forms of FVIIIXTEN HBoV1 ceDNA (ceFVIIIXTEN) in hFVIIIR593C^{+/+}/HemA mice via hydrodynamic tail-vein injections.

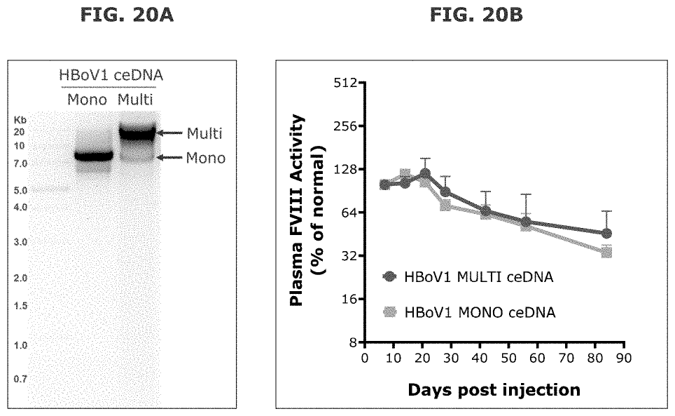

Monomeric and multimeric forms of ceFVIIIXTEN were generated by PAGE purification, as described previously (see International Application No. PCT/US2021/047218). The quality of concatameric forms of ceFVIIIXTEN was determined by 0.8 to 1.2% agarose gel electrophoresis and results showed the majority of species were either the monomeric or multimeric form of ceFVIIIXTEN (FIG. 20A). Purified monomeric or multimeric ceFVIIIXTEN were systemically injected in hFVIIIR593C^{+/+}/HemA mice via hydrodynamic tail-vein injections at 40 μg/kg. Plasma samples were collected from injected mice at 7 day intervals for about 3 months. Plasma FVIII activity was measured by the Chromogenix Coatest® SP Factor VIII chromogenic assay, according to the manufacturer's instructions.

The plasma FVIII activity normalized to percent of normal for ceFVIIIXTEN injected animals is shown in FIG. 20B. The results showed no significant difference in FVIII expression levels between monomeric or multimeric forms of ceFVIIIXTEN over the course of 3 months. This data suggests that both monomeric and multimeric forms of ceFVIIIXTEN have comparable in vivo potency and stability.

### Example 17: FVIIIXTEN HBoV1 mTTR vs A1AT ssDNA In Vivo Efficacy

The FVIIIXTEN expression cassette used in the experiments disclosed above contains a mTTR promoter and enhancer element (V2.0, FIG. 1). This promoter is mouse-liver specific, but its liver-specific expression has not been studied in large animal models or in human subjects. Therefore, in this study, the V3.0 FVIIIXTEN expression cassette (SEQ ID NO: 35) was generated by replacing the mTTR promoter and enhancer element with human liver-specific alpha-1-antitrypsin (A1AT) promoter (SEQ ID NO: 36) in the V2.0 expression cassette (FIG. 1).

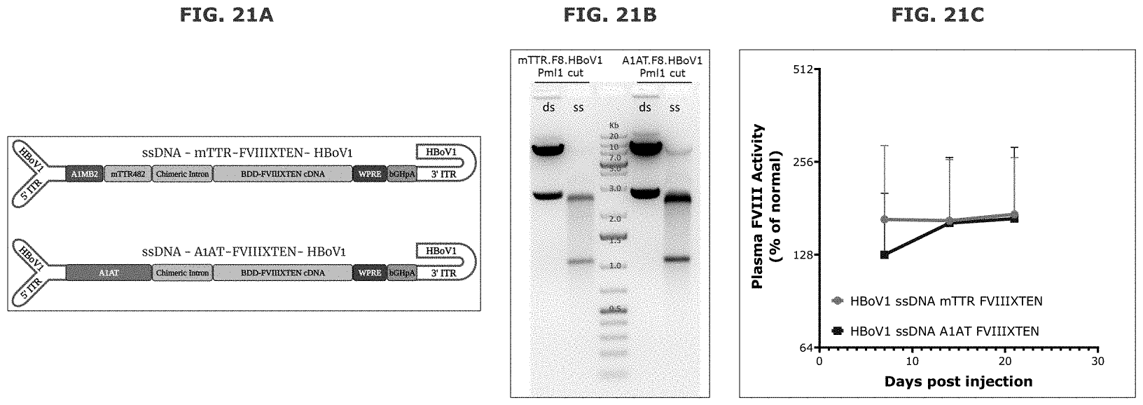

To validate the functionality of the mTTR versus the A1AT promoter in vivo, single-stranded DNA (ssDNA) comprising codon-optimized human FVIIIXTEN (ssFVIIIXTEN) with preformed HBoV1 ITRs was tested in hFVIIIR593C^{+/+}/HemA mice (FIG. 21A). The ssFVIIIXTEN with preformed HBoV1 ITRs was generated by denaturing the double-stranded DNA (dsDNA) fragment products (mTTR or A1AT FVIII expression cassette and plasmid backbone) of PmII digestion at 95° C. and then cooling down at 4° C. to allow the palindromic ITR sequences to fold. The resulting ssFVIIIXTEN was checked by 0.8 to 1.2% agarose gel electrophoresis. The gel analysis showed half the size of dsDNA for ssFVIIIXTEN suggesting efficient hairpin formation (FIG. 21B). The ssFVIIIXTEN was systemically injected into hFVIIIR593C^{+/+}/HemA mice via hydrodynamic tail-vein injections at 10 μg/mouse. Plasma samples were collected from injected mice at 7 day intervals for 5.5 months. Plasma FVIII activity was measured by the Chromogenix Coatest® SP Factor VIII chromogenic assay, according to the manufacturer's instructions.

The plasma FVIII activity normalized to percent of normal for ssFVIIIXTEN injected animals is shown in FIG. 21C. These results showed equivalent levels of FVIII expression up to day 21 post-injection, suggesting there is no significant difference in FVIIIXTEN levels expressed by the mTTR or AAT promoter in hFVIIIR593C^{+/+}/HemA mice animal model.

TABLE 2
SEQUENCES
Additional Nucleotide and Amino Acid Sequences

| SEQ ID NO/ | |
|---|---|
| Description | Nucleotide or amino acid sequence |
| SEQ ID NO. 1: | GTGGTTGTACAGACGCCATCTTGGAATCCAATATGTCTGCCGGCTCAGTCATGCCTGCGCTGCGCGCAGCGCGCTGC |
| HBoV1 5′ ITR | GCGCGCGCATGATCTAATCGCCGGCAGACATATTGGATTCCAAGATGGCGTCTGTACAACCAC |
| SEQ ID NO. 2: | TTGCTTATGCAATCGCGAAACTCTATATCTTTTAATGTGTTGTTGTTGTACATGCGCCATCTTAGTTTTATATCAGC |
| HBoV1 3′ ITR | TGGCGCCTTAGTTATATAACATGCATGTTATATAACTAAGGCGCCAGCTGATATAAAACTAAGATGGCGCATGTACA |
| | ACAACAACACATTAAAAGATATAGAGTTTCGCGATTGCATAAGCAA |
| SEQ ID NO. 3: | GTATACCTGCAGGCTAGCCACGTGTTGTTGTTGTACATGCGCCATCTTAGTTTTATATCAGCTGGCGCCTTAGTTAT |
| HBoV1-5′ITR- | ATAACATGCATGTTATATAACTAAGGCGCCAGCTGATATAAAACTAAGATGGCGCATGTACAACAACAACACATTAA |
| mTTR482- | AAGATATAGAGTTTCGCGATTGCAAGCTTGGCCCCAGGTTAATTTTTAAAAAGCAGTCAAAGGTCAAAGTGGCCCTT |
| Intron- | GGCAGCATTTACTCTCTCTATTGACTTTGGTTAATAATCTCAGGAGCACAAACATTCCTGGAGGCAGGAGAAGAAAT |
| coBDDFVIIIX | CAACATCCTGGACTTATCCTCTGGGCCTCTCCCCACCTTCGATGGCCCCAGGTTAATTTTTAAAAAGCAGTCAAAGG |
| TEN (V2.0)- | TCAAAGTGGCCCTTGGCAGCATTTACTCTCTCTATTGACTTTGGTTAATAATCTCAGGAGCACAAACATTCCTGGAG |
| WPRE- | GCAGGAGAAGAAATCAACATCCTGGACTTATCCTCTGGGCCTCTCCCCACCGATATCTACCTGCTGATCGCCCGGCC |
| bGHPolyA- | CCTGTTCAAACATGTCCTAATACTCTGTCGGGGCAAAGGTCGGCAGTAGTTTTCCATCTTACTCAACATCCTCCCAG |
| HBoV1-3′ITR | TGTACGTAGGATCCTGTCTGTCTGCACATTTCGTAGAGCGAGTGTTCCGATACTCTAATCTCCCGGGGCAAAGGTCG |
| | TATTGACTTAGGTTACTTATTCTCCTTTTGTTGACTAAGTCAATAATCAGAATCAGCAGGTTTGGAGTCAGCTTGGC |
| | AGGGATCAGCAGCCTGGGTTGGAAGGAGGGGGTATAAAAGCCCCTTCACCAGGAGAAGCCGTCACACAGATCCACAA |
| | GCTCCTGCTAGGAATTCTCAGGAGCACAAACATTCCTGGAGGCAGGAGAAGAAATCAACATCCTGGACTTATCCTCT |
| | GGGCCTCTCCCCACCGATATCTACCTGCTGATCGCCCGGCCCCTGTTCAAACATGTCCTAATACTCTGTCGGGGCAA |
| | AGGTCGGCAGTAGTTTTCCATCTTACTCAACATCCTCCCAGTGTACGTAGGATCCTGTCTGTCTGCACATTTCGTAG |
| | AGCGAGTGTTCCGATACTCTAATCTCCCGGGGCAAAGGTCGTATTGACTTAGGTTACTTATTCTCCTTTTGTTGACT |
| | AAGTCAATAATCAGAATCAGCAGGTTTGGAGTCAGCTTGGCAGGGATCAGCAGCCTGGGTTGGAAGGAGGGGGTATA |
| | AAAGCCCCTTCACCAGGAGAAGCCGTCACACAGATCCACAAGCTCCTGCTAGAGTCGCTGCGCGCTGCCTTCGCCCC |
| | GTGCCCCGCTCCGCCGCCGCCTCGCGCCGCCCGCCCCGGCTCTGACTGACCGCGTTACTCCCACAGGTGAGCGGGCG |
| | GGACGGCCCTTCTCCTCCGGGCTGTAATTAGCGCTTGGTTTATTGACGGCTTGTTTCTTTTCTGTGGCTGCGTGAAA |
| | GCCTTGAGGGGCTCCGGGAAGGCCCTTTGTGCGGGGGGAGCGGCTCGGGGGGTGCGTGCGTGTGTGTGTGCGTGGGG |
| | AGCGCCGCGTGCGGCTCCGCGCTGCCCGGCGGCTGTGAGCGCTGCGGGCGCGGCGCGGGGCTTTGTGCGCTCCGCAG |
| | TGTGCGCGAGGGGAGCGCGGCCGGGGGCGGTGCCCCGCGGTGCGGGGGGGGCTGCGAGGGGAACAAAGGCTGCGTGC |
| | GGGGTGTGTGCGTGGGGGGGTGAGCAGGGGGTGTGGGCGCGTCGGTCGGGCTGCAACCCCCCCTGCACCCCCCTCCC |
| | CGAGTTGCTGAGCACGGCCCGGCTTCGGGTGCGGGGCTCCGTACGGGGCGTGGCGCGGGGCTCGCCGTGCCGGGCGG |
| | GGGGTGGCGGCAGGTGGGGGTGCCGGGCGGGGCGGGGCCGCCTCGGGCCGGGGAGGGCTCGGGGGAGGGGCGCGGCG |
| | GCCCCCGGAGCGCCGGCGGCTGTCGAGGCGCGGCGAGCCGCAGCCATTGCCTTTTATGGTAATCGTGCGAGAGGGCG |
| | CAGGGACTTCCTTTGTCCCAAATCTGTGCGGAGCCGAAATCTGGGAGGCGCCGCCGCACCCCCTCTAGCGGGCGCGG |
| | GGCGAAGCGGTGCGGCGCCGGCAGGAAGGAAATGGGCGGGGAGGGCCTTCGTGCGTCGCCGCGCCGCCGTCCCCTTC |
| | TCCCTCTCCAGCCTCGGGGCTGTCCGCGGGGGGACGGCTGCCTTCGGGGGGGACGGGGCAGGGCGGGGTTCGGCTTC |
| | TGGCGTGTGACCGGCGGCTCTAGAGCCTCTGCTAACCTTGTTCTTGCCTTCTTCTTTTTCCTACAGCTCCTGGGCAA |
| | CGTGCTGGTTATTGTGCTGTCTCATCATTTTGGCAAAGAATTACTCGAGGCCACCATGCAGATTGAACTGTCCACTT |
| | GCTTCTTCCTGTGCCTCCTGCGGTTTTGCTTCTCGGCCACCCGCCGGTATTACTTAGGTGCTGTGGAACTGAGCTGG |
| | GACTACATGCAGTCCGACCTGGGAGAACTGCCGGTGGACGCGAGATTCCCACCTAGAGTCCCGAAGTCCTTCCCATT |
| | CAACACCTCCGTGGTCTACAAAAAGACCCTGTTCGTGGAGTTCACTGACCACCTTTTCAATATTGCCAAGCCGCGCC |
| | CCCCCTGGATGGGCCTGCTTGGTCCTACGATCCAAGCAGAGGTCTACGACACCGTGGTCATCACACTGAAGAACATG |
| | GCCTCACACCCCGTGTCGCTGCATGCTGTGGGAGTGTCCTACTGGAAGGCCTCAGAGGGTGCCGAATATGATGACCA |
| | GACCAGCCAGAGGGAAAAGGAGGATGACAAAGTGTTCCCGGGTGGCAGCCACACTTACGTGTGGCAAGTGCTGAAGG |
| | AAAACGGGCCTATGGCGTCGGACCCCCTATGCCTGACCTACTCCTACCTGTCCCATGTGGACCTTGTGAAGGATCTC |
| | AACTCGGGACTGATCGGCGCCCTCTTGGTGTGCAGAGAAGGCAGCCTGGCGAAGGAAAAGACTCAGACCCTGCACAA |
| | GTTCATTCTGTTGTTTGCTGTGTTCGATGAAGGAAAGTCCTGGCACTCAGAAACCAAGAACTCGCTGATGCAGGATA |
| | GAGATGCGGCCTCGGCCAGAGCCTGGCCTAAAATGCACACCGTCAACGGATATGTGAACAGGTCGCTCCCTGGCCTC |
| | ATCGGCTGCCACAGAAAGTCCGTGTATTGGCATGTGATCGGCATGGGTACTACTCCGGAAGTGCATAGTATCTTTCT |
| | GGAGGGCCATACCTTCTTGGTGCGCAACCACAGACAGGCCTCGCTGGAAATCTCGCCTATCACTTTCTTGACTGCGC |
| | AGACCCTCCTTATGGACCTTGGACAGTTCCTGCTGTTCTGTCACATCAGCTCCCATCAGCATGATGGGATGGAGGCC |
| | TATGTCAAAGTGGACTCCTGCCCTGAGGAGCCACAGCTCCGGATGAAGAACAATGAGGAAGCGGAGGATTACGACGA |
| | CGACCTGACTGACAGCGAAATGGACGTCGTGCGATTCGATGACGACAACAGCCCGTCCTTCATCCAAATTAGATCAG |
| | TGGCGAAGAAGCACCCCAAGACCTGGGTGCACTACATTGCCGCCGAGGAAGAGGACTGGGACTACGCGCCGCTGGTG |
| | CTGGCGCCAGACGACAGGAGCTACAAGTCCCAGTACCTCAACAACGGGCCGCAGCGCATTGGCAGGAAGTACAAGAA |
| | AGTCCGCTTCATGGCCTACACTGATGAAACCTTCAAGACGAGGGAAGCCATCCAGCACGAGTCAGGCATCCTGGGAC |
| | CGCTCCTTTACGGCGAAGTCGGGGATACCCTGCTCATCATTTTCAAGAACCAGGCATCGCGGCCCTACAACATCTAC |
| | CCTCACGGGATCACAGACGTGCGCCCGCTCTACTCCCGCCGGCTGCCCAAGGGAGTGAAGCACCTGAAGGATTTTCC |
| | CATCCTGCCGGGAGAAATCTTCAAGTACAAGTGGACCGTGACTGTGGAAGATGGCCCTACCAAGTCGGACCCTCGCT |
| | GTCTGACCCGGTACTATTCCTCGTTTGTGAACATGGAGCGCGACCTGGCCTCGGGGCTGATTGGTCCGCTGCTGATC |
| | TGCTACAAGGAGTCCGTGGACCAGCGCGGGAACCAGATCATGTCCGACAAGCGCAACGTGATCCTGTTCTCTGTCTT |
| | TGATGAAAACAGATCGTGGTACTTGACTGAGAATATCCAGCGGTTCCTGCCCAACCCAGCGGGAGTGCAACTGGAGG |
| | ACCCGGAGTTCCAGGCCTCAAACATTATGCACTCTATCAACGGCTATGTGTTCGACTCGCTCCAACTGAGCGTGTGC |
| | CTGCATGAAGTGGCATACTGGTACATTCTGTCCATCGGAGCCCAGACCGACTTCCTGTCCGTGTTCTTCTCCGGATA |
| | CACCTTCAAGCATAAGATGGTGTACGAGGACACTCTGACCCTCTTCCCATTTTCCGGAGAAACTGTGTTCATGTCAA |
| | TGGAAAACCCGGGCTTGTGGATTCTGGGTTGCCATAACTCGGACTTCCGGAATAGAGGGATGACCGCCCTGCTGAAA |
| | GTGTCCAGCTGTGACAAGAATACCGGCGATTACTACGAGGACAGCTATGAGGACATCTCCGCTTATCTGCTGTCCAA |
| | GAACAACGCCATTGAACCCAGGTCCTTCTCCCAAAACGGTGCACCGACCTCCGAAAGCGCCACCCCAGAGTCAGGAC |
| | CTGGCTCGGAACCGGCTACCTCGGGCTCAGAGACACCGGGGACTTCCGAGTCCGCAACCCCCGAGAGTGGACCCGGA |
| | TCCGAACCAGCAACCTCAGGATCAGAAACCCCGGGAACTTCGGAATCCGCCACTCCCGAGTCGGGACCAGGCACCTC |
| | CACTGAGCCTTCCGAGGGAAGCGCCCCCGGATCCCCTGCTGGATCCCCTACCAGCACTGAAGAAGGCACCTCAGAAT |
| | CCGCGACCCCTGAGTCCGGCCCTGGAAGCGAACCCGCCACCTCCGGTTCCGAAACCCCTGGGACTAGCGAGAGCGCC |
| | ACTCCGGAATCGGGCCCAGGAAGCCCTGCCGGATCCCCGACCAGCACCGAGGAGGGAAGCCCCGCCGGGTCACCGAC |
| | TTCCACTGAGGAGGGAGCCTCATCCCCCCCCGTGCTGAAGCGGCATCAAAGAGAGATCACCAGGACCACTCTCCAGT |
| | CCGATCAGGAAGAAATTGACTACGACGATACTATCAGCGTGGAGATGAAGAAGGAGGACTTCGACATCTACGATGAG |
| | GATGAGAACCAGTCCCCTCGGAGCTTTCAGAAGAAAACCCGCCACTACTTCATCGCTGCCGTGGAGCGGCTGTGGGA |
| | TTACGGGATGTCCAGCTCACCGCATGTGCTGCGGAATAGAGCGCAGTCAGGATCGGTGCCCCAGTTCAAGAAGGTCG |
| | TGTTCCAAGAGTTCACCGACGGGTCCTTCACTCAACCCCTGTACCGGGGCGAACTCAACGAACACCTGGGACTGCTT |
| | GGGCCGTATATCAGGGCAGAAGTGGAAGATAACATCATGGTCACCTTCCGCAACCAGGCCTCCCGGCCGTACAGCTT |
| | CTACTCTTCACTGATCTCCTACGAGGAAGATCAGCGGCAGGGAGCCGAGCCCCGGAAGAACTTCGTCAAGCCTAACG |
| | AAACTAAGACCTACTTTTGGAAGGTCCAGCATCACATGGCCCCGACCAAAGACGAGTTCGACTGTAAAGCCTGGGCC |
| | TACTTCTCCGATGTGGACCTGGAGAAGGACGTGCACTCGGGACTCATTGGCCCGCTCCTTGTGTGCCATACTAATAC |
| | CCTGAACCCTGCTCACGGTCGCCAAGTCACAGTGCAGGAGTTCGCCCTCTTCTTCACCATCTTCGATGAAACAAAGT |
| | CCTGGTACTTTACTGAGAACATGGAACGCAATTGCAGGGCACCCTGCAACATCCAGATGGAAGATCCCACCTTCAAG |
| | GAAAACTACCGGTTTCATGCCATTAACGGCTACATAATGGACACGTTGCCAGGACTGGTCATGGCCCAGGACCAGAG |
| | AATCCGGTGGTATCTGCTCTCCATGGGCTCCAACGAAAACATTCACAGCATTCATTTTTCCGGCCATGTGTTCACCG |
| | TCCGGAAGAAGGAAGAGTACAAGATGGCTCTGTACAACCTCTACCCTGGAGTGTTCGAGACTGTGGAAATGCTGCCT |
| | AGCAAGGCCGGCATTTGGAGAGTGGAATGCCTGATCGGAGAGCATTTGCACGCCGGAATGTCCACCCTGTTTCTTGT |
| | GTACTCCAACAAGTGCCAGACCCCGCTGGGAATGGCCTCAGGTCATATTAGGGATTTCCAGATCACTGCTTCGGGGC |
| | AGTACGGGCAGTGGGCACCTAAGTTGGCCCGGCTGCACTACTCTGGCTCCATCAATGCCTGGTCCACCAAGGAACCC |
| | TTCTCCTGGATTAAGGTGGACCTCCTGGCCCCAATGATTATTCACGGTATTAAGACCCAGGGTGCCCGACAGAAGTT |
| | CTCCTCACTCTACATCTCGCAATTCATCATAATGTACAGCCTGGATGGGAAGAAGTGGCAGACCTACCGGGGAAACT |
| | CCACTGGAACGCTCATGGTGTTTTTCGGCAACGTGGACTCCTCCGGCATTAAGCACAACATCTTCAACCCTCCGATC |
| | ATTGCTCGGTACATCCGGCTGCACCCAACTCACTACAGCATCCGGTCCACCCTGCGGATGGAACTGATGGGTTGTGA |
| | CCTGAACTCCTGCTCCATGCCCCTTGGGATGGAATCCAAGGCCATTAGCGATGCACAGATCACCGCCTCTTCATACT |
| | TCACCAACATGTTCGCGACCTGGTCCCCGTCGAAGGCCCGCCTGCACCTCCAAGGTCGCTCCAATGCGTGGCGGCCT |
| | CAAGTGAACAACCCCAAGGAGTGGCTCCAGGTCGACTTCCAAAAGACCATGAAGGTCACCGGAGTGACCACCCAGGG |
| | CGTGAAGTCCCTGCTGACCTCTATGTACGTTAAGGAGTTCCTCATCTCCTCAAGCCAAGACGGACATCAGTGGACCC |
| | TGTTCTTCCAAAACGGAAAAGTCAAAGTATTCCAGGGCAACCAGGACTCCTTCACCCCTGTGGTCAACAGCCTGGAC |
| | CCCCCATTGCTGACCCGCTACCTCCGCATCCACCCCCAAAGCTGGGTCCACCAGATCGCACTGCGCATGGAGGTCCT |
| | TGGATGCGAAGCCCAAGATCTGTACTAAGCGGCCGCTCATAATCAACCTCTGGATTACAAAATTTGTGAAAGATTGA |
| | CTGGTATTCTTAACTATGTTGCTCCTTTTACGCTATGTGGATACGCTGCTTTAATGCCTTTGTATCATGCTATTGCT |
| | TCCCGTATGGCTTTCATTTTCTCCTCCTTGTATAAATCCTGGTTGCTGTCTCTTTATGAGGAGTTGTGGCCCGTTGT |
| | CAGGCAACGTGGCGTGGTGTGCACTGTGTTTGCTGACGCAACCCCCACTGGTTGGGGCATTGCCACCACCTGTCAGC |
| | TCCTTTCCGGGACTTTCGCTTTCCCCCTCCCTATTGCCACGGCGGAACTCATCGCCGCCTGCCTTGCCCGCTGCTGG |
| | ACAGGGGCTCGGCTGTTGGGCACTGACAATTCCGTGGTGTTGTCGGGGAAATCATCGTCCTTTCCTTGGCTGCTCGC |
| | CTGTGTTGCCACCTGGATTCTGCGCGGGACGTCCTTCTGCTACGTCCCTTCGGCCCTCAATCCAGCGGACCTTCCTT |
| | CCCGCGGCCTGCTGCCGGCTCTGCGGCCTCTTCCGCGTCTTCGCCTTCGCCCTCAGACGAGTCGGATCTCCCTTTGG |
| | GCCGCCTCCCCGCTGCCTAGGCGACTGTGCCTTCTAGTTGCCAGCCATCTGTTGTTTGCCCCTCCCCCGTGCCTTCC |
| | TTGACCCTGGAAGGTGCCACTCCCACTGTCCTTTCCTAATAAAATGAGGAAATTGCATCGCATTGTCTGAGTAGGTG |
| | TCATTCTATTCTGGGGGGTGGGGTGGGGCAGGACAGCAAGGGGGAGGATTGGGAAGACAATAGCAGGCATGCTGGGG |
| | AAGACCATGGGCGCGCCAGGCCTGTCGACGCCCGGGCGGTACCGCGATCGCTCGCGACGCATAAAGTATATGTGACG |
| | TGGTTGTACAGACGCCATCTTGGAATCCAATATGTCTGCCGGCGATTAGATCATGCGCGCGCGCAGCGCGCTGCGCG |
| | CAGCGCAGGCATGACTGAGCCGGCAGACATATTGGATTCCAAGATGGCGTCTGTACAACCACGTGCTTAAGCTGCAG |
| | ACTAGTGAGCTCGTTAAC |
| SEQ ID NO. 4: | GCGGCCGCGGATCCGCCACCATGGCATTCAATCCGCCCGTAATACGCGCATTTTCACAACCCGCCTTTACGTATGTC |
| Sf codon | TTTAAGTTTCCGTACCCTCAATGGAAAGAGAAAGAGTGGCTACTGCACGCGTTGCTTGCCCACGGCACCGAGCAGTC |
| optimized | CATGATTCAATTACGTAACTGTGCCCCACACCCGGACGAGGATATTATCCGGGACGATCTTCTAATAAGTTTGGAAG |
| HBoV1 NS1 | ATAGGCATTTCGGGGCGGTCCTGTGTAAAGCGGTATACATGGCTACTACCACGTTGATGTCTCACAAGCAACGCAAT |
| | ATGTTCCCAAGGTGCGACATAATCGTTCAGTCAGAGTTAGGTGAAAAAAATTTACATTGTCATATTATCGTTGGAGG |
| | CGAAGGCCTATCAAAGAGAAACGCTAAGAGCTCTTGCGCTCAGTTTTACGGACTTATATTAGCAGAAATTATCCAGC |
| | GCTGTAAGAGTTTACTAGCCACCCGTCCGTTTGAGCCGGAAGAAGCGGATATATTTCATACGTTGAAGAAAGCGGAG |
| | CGCGAGGCCTGGGGTGGAGTTACTGGCGGTAACATGCAAATCTTACAATACAGGGACCGTCGGGGTGACCTGCATGC |
| | ACAGACTGTTGATCCCCTCAGATTCTTCAAAAATTATTTGTTACCGAAGAACCGATGCATAAGTAGTTACAGCAAAC |
| | CTGATGTCTGTACTAGCCCTGATAACTGGTTCATTCTGGCCGAAAAAACGTACTCGCATACACTTATCAATGGATTG |
| | CCGCTTCCCGAGCACTATCGAAAAAACTATCATGCCACCCTGGATAATGAAGTTATACCTGGACCACAGACTATGGC |
| | GTATGGAGGGAGAGGCCCTTGGGAACATTTACCCGAGGTGGGTGACCAGAGGCTTGCCGCAAGTTCCGTGAGCACTA |
| | CGTATAAGCCAAACAAGAAGGAGAAGCTAATGCTCAACCTCCTCGACAAGTGTAAGGAGTTGAATCTTCTAGTTTAT |
| | GAGGATCTTGTAGCGAACTGCCCAGAGCTGCTGCTCATGCTAGAAGGCCAACCTGGAGGTGCTCGACTCATCGAGCA |
| | AGTACTAGGAATGCATCACATCAATGTATGCTCGAATTTCACCGCGCTAACGTACCTCTTCCATCTGCATCCGGTGA |
| | CATCGCTGGATAGTGACAACAAAGCGTTACAGCTTTTACTAATTCAAGGGTACAACCCCCTGGCAGTGGGGCATGCT |
| | CTCTGTTGTGTGTTAAACAAACAATTTGGTAAACAGAACACAGTCTGTTTTTACGGGCCAGCATCTACTGGGAAAAC |
| | AAATATGGCAAAAGCGATTGTGCAGGGAATCCGGCTATATGGCTGCGTCAACCATCTTAACAAGGGTTTTGTTTTCA |
| | ATGATTGTCGACAACGCCTCGTAGTCTGGTGGGAGGAATGCCTAATGCACCAGGACTGGGTGGAGCCAGCAAAGTGT |
| | ATTCTTGGCGGGACCGAATGTCGTATCGACGTCAAGCACAGAGATTCTGTCCTATTGACACAAACGCCTGTAATAAT |
| | TTCGACTAATCACGACATTTACGCCGTCGTGGGAGGGAATTCGGTGTCTCACGTTCACGCTGCGCCTCTCAAAGAAC |
| | GGGTTATTCAGCTGAATTTTATGAAACAACTCCCCCAAACTTTTGGTGAGATAACCGCCACAGAAATCGCTGCTCTG |
| | CTACAGTGGTGCTTTAATGAATATGACTGCACCCTGACAGGTTTCAAACAGAAGTGGAATTTGGACAAGATACCTAA |
| | CTCATTCCCGTTGGGGGTATTGTGCCCAACACATTCCCAAGATTTCACACTTCACGAAAATGGGTATTGCACGGACT |
| | GCGGGGGCTACCTTCCCCACTCCGCTGATAATTCAATGTATACCGATCGGGCTAGCGAAACATCCACCGGCGACATA |
| | ACGCCCTCCAAATGATTCGAATCTAGAGCCTGCAGTCTCGAGGCATGCGGTACC |
| SEQ ID NO. 5: | GTGGACGTGAAAGAAACC |
| Outside Primer | |
| SEQ ID NO. 6: | GGTCATAGCTGTTTCCTGTG |
| Inside Primer | |
| SEQ ID NO. 7: | ATTAAGCTTCCGCGTAAAACACAATCAAGTATGAGTCATAAGCTGATGTCATGTTTTGCACACGGCTCATAACCGAA |
| hr5.ie1.neo.p | CTGGCTTTACGAGTAGAATTCTACTTGTAACGCACGATCAGTGGATGATGTCATTTGTTTTTCAAATCGAGATGATG |
| 10PAS | TCATGTTTTGCACACGGCTCATAAACTCGCTTTACGGGTAGAATTCTACGTGTAACGCACGATCGATTGATGAGTCA |
| | TTTGTTTTGCAATATGATATCATACAATATGACTCATTTGTTTTTCAAAACCGAACTTGATTTACGGGTAGAATTCT |
| | ACTTGTAAAGCACAATCAAAAAGATGATGTCATTTGTTTTTCAAAACTGAACTCGCTTTACGAGTAGAATTCTACGT |
| | GTAAAACACAATCAAGAAATGATGTCATTTGTTATAAAAATAAAAGCTGATGTCATGTTTTGCACATGGCTCATAAC |
| | TAAACTCGCTTTACGGGTAGAATTCTACGCGCGTCGATGTCTTTGTGATGCGCGCGACATTTTTGTAGGTTATTGAT |
| | AAAATGAACGGATACGTTGCCCGACATTATCATTAAATCCTTGGCGTAGAATTTGTCGGGTCCATTGTCCGTGTGCG |
| | CTAGCATGCCCGTAACGGACCTCGTACTTTTGGCTTCAAAGGTTTTGCGCACAGACAAAATGTGCCACACTTGCAGC |
| | TCTGCATGTGTGCGCGTTACCACAAATCCCAACGGCGCAGTGTACTTGTTGTATGCAAATAAATCTCGATAAAGGCG |
| | CGGCGCGCGAATGCAGCTGATCACGTACGCTCCTCGTGTTCCGTTCAAGGACGGTGTTATCGACCTCAGATTAATGT |
| | TTATCGGCCGACTGTTTTCGTATCCGCTCACCAAACGCGTTTTTGCATTAACATTGTATGTCGGCGGATGTTCTATA |
| | TCTAATTTGAATAAATAAACGATAACCGCGTTGGTTTTAGAGGGCATAATAAAAGAAATATTGTTATCGTGTTCGCC |
| | ATTAGGGCAGTATAAATTGACGTTCATGTTGGATATTGTTTCAGTTGCAAGTTGACACTGGCGGCGACAAGATCGTG |
| | AACAACCAAGTGACGCGGCCGCATTTGTAAAAAAAAAATAAATAAAAATGATCGAGCAGGACGGCCTGCACGCTGGT |
| | TCTCCAGCTGCTTGGGTCGAGCGTCTGTTCGGTTACGACTGGGCTCAGCAGACCATCGGTTGCTCCGACGCTGCTGT |
| | GTTCCGTCTGTCCGCTCAGGGTCGTCCCGTGCTGTTCGTCAAGACCGACCTGTCCGGTGCTCTGAACGAGCTGCAGG |
| | ACGAGGCTGCTCGTCTGTCCTGGCTGGCTACCACTGGTGTCCCTTGCGCTGCTGTCCTGGACGTGGTCACTGAGGCT |
| | GGTCGTGACTGGCTGCTGCTGGGAGAAGTGCCTGGACAGGACCTGCTGTCCAGCCACCTGGCTCCAGCTGAGAAGGT |
| | GTCCATCATGGCTGACGCTATGCGTCGTCTGCACACCCTGGACCCTGCTACCTGCCCCTTCGACCACCAAGCTAAGC |
| | ACCGTATCGAGCGTGCTCGTACCCGTATGGAAGCTGGCCTGGTGGACCAGGACGACCTGGACGAAGAACACCAGGGA |
| | CTGGCCCCTGCTGAGCTGTTCGCTCGTCTGAAGGCTCGTATGCCCGACGGCGAGGACCTGGTGGTTACTCACGGCGA |
| | CGCTTGCCTGCCCAACATCATGGTCGAGAACGGTCGTTTCTCCGGTTTCATCGACTGCGGTCGTCTGGGTGTCGCTG |
| | ACCGTTACCAGGATATCGCTCTGGCTACCCGTGATATCGCTGAGGAACTGGGTGGCGAGTGGGCTGACAGATTCCTG |
| | GTGCTGTACGGTATCGCTGCTCCCGACTCCCAGCGTATCGCTTTCTACCGTCTGCTGGACGAGTTCTTCTAAGCCCC |
| | TTGTAAACGCCACAATTGTGTTTGTTGCAAATAAACCCATGATTATTTGATTAAAATTGTTGTTTTCTTTGTTCATA |
| | GACAATAGTGTGTTTTGCCTAAACGGGTACC |
| SEQ ID NO. 8: | ATTAAGCTTCCGCGTAAAACACAATCAAGTATGAGTCATAAGCTGATGTCATGTTTTGCACACGGCTCATAACCGAA |
| hr5.ie1.eGFP. | CTGGCTTTACGAGTAGAATTCTACTTGTAACGCACGATCAGTGGATGATGTCATTTGTTTTTCAAATCGAGATGATG |
| p10PAS | TCATGTTTTGCACACGGCTCATAAACTCGCTTTACGGGTAGAATTCTACGTGTAACGCACGATCGATTGATGAGTCA |
| | TTTGTTTTGCAATATGATATCATACAATATGACTCATTTGTTTTTCAAAACCGAACTTGATTTACGGGTAGAATTCT |
| | ACTTGTAAAGCACAATCAAAAAGATGATGTCATTTGTTTTTCAAAACTGAACTCGCTTTACGAGTAGAATTCTACGT |
| | GTAAAACACAATCAAGAAATGATGTCATTTGTTATAAAAATAAAAGCTGATGTCATGTTTTGCACATGGCTCATAAC |
| | TAAACTCGCTTTACGGGTAGAATTCTACGCGCGTCGATGTCTTTGTGATGCGCGCGACATTTTTGTAGGTTATTGAT |
| | AAAATGAACGGATACGTTGCCCGACATTATCATTAAATCCTTGGCGTAGAATTTGTCGGGTCCATTGTCCGTGTGCG |
| | CTAGCATGCCCGTAACGGACCTCGTACTTTTGGCTTCAAAGGTTTTGCGCACAGACAAAATGTGCCACACTTGCAGC |
| | TCTGCATGTGTGCGCGTTACCACAAATCCCAACGGCGCAGTGTACTTGTTGTATGCAAATAAATCTCGATAAAGGCG |
| | CGGCGCGCGAATGCAGCTGATCACGTACGCTCCTCGTGTTCCGTTCAAGGACGGTGTTATCGACCTCAGATTAATGT |
| | TTATCGGCCGACTGTTTTCGTATCCGCTCACCAAACGCGTTTTTGCATTAACATTGTATGTCGGCGGATGTTCTATA |
| | TCTAATTTGAATAAATAAACGATAACCGCGTTGGTTTTAGAGGGCATAATAAAAGAAATATTGTTATCGTGTTCGCC |
| | ATTAGGGCAGTATAAATTGACGTTCATGTTGGATATTGTTTCAGTTGCAAGTTGACACTGGCGGCGACAAGATCGTG |
| | AACAACCAAGTGACGCGGCCGCATTTGTAAAAAAAAAATAAATAAAAATGGTGTCCAAGGGCGAGGAACTGTTCACC |
| | GGTGTCGTGCCCATCCTGGTCGAACTGGACGGCGACGTGAACGGTCACAAGTTCTCCGTGTCTGGCGAAGGCGAGGG |
| | CGACGCTACCTACGGAAAGCTGACCCTGAAGTTCATCTGCACCACCGGCAAGCTGCCCGTGCCTTGGCCTACCCTGG |
| | TCACCACTCTGACCTACGGTGTCCAGTGCTTCTCCCGTTACCCCGACCACATGAAGCAGCACGATTTCTTCAAGTCC |
| | GCTATGCCCGAGGGTTACGTGCAAGAGCGTACCATCTTCTTCAAGGACGACGGCAACTACAAGACCCGTGCTGAAGT |
| | GAAGTTCGAAGGCGACACCCTCGTGAACCGTATCGAGCTGAAGGGTATCGACTTCAAGGAAGATGGAAACATCCTGG |
| | GCCACAAGCTCGAGTACAACTACAACTCCCACAACGTGTACATCATGGCCGACAAGCAAAAGAACGGCATCAAAGTG |
| | AACTTCAAGATCCGCCACAACATCGAGGACGGTTCCGTGCAGCTGGCTGACCACTACCAGCAGAACACCCCCATCGG |
| | CGACGGTCCTGTGCTGCTGCCTGACAACCACTACCTGTCCACCCAGTCCGCTCTGTCCAAGGACCCCAACGAGAAGC |
| | GTGACCACATGGTGCTGCTCGAGTTCGTGACCGCTGCTGGTATCACCCTGGGCATGGACGAGCTGTACAAGTAAGCC |
| | CCTTGTAAACGCCACAATTGTGTTTGTTGCAAATAAACCCATGATTATTTGATTAAAATTGTTGTTTTCTTTGTTCA |
| | TAGACAATAGTGTGTTTTGCCTAAACGGGTACC |
| SEQ ID NO: 9 | ATGCAGATTGAACTGTCCACTTGCTTCTTCCTGTGCCTCCTGCGGTTTTGCTTCTCGGCCACCCGCCGGTATTACTT |
| Nucleotide | AGGTGCTGTGGAACTGAGCTGGGACTACATGCAGTCCGACCTGGGAGAACTGCCGGTGGACGCGAGATTCCCACCTA |
| sequence | GAGTCCCGAAGTCCTTCCCATTCAACACCTCCGTGGTCTACAAAAAGACCCTGTTCGTGGAGTTCACTGACCACCTT |
| encoding | TTCAATATTGCCAAGCCGCGCCCCCCCTGGATGGGCCTGCTTGGTCCTACGATCCAAGCAGAGGTCTACGACACCGT |
| coBDDFVIIIX | GGTCATCACACTGAAGAACATGGCCTCACACCCCGTGTCGCTGCATGCTGTGGGAGTGTCCTACTGGAAGGCCTCAG |
| TEN (V2.0) | AGGGTGCCGAATATGATGACCAGACCAGCCAGAGGGAAAAGGAGGATGACAAAGTGTTCCCGGGTGGCAGCCACACT |
| | TACGTGTGGCAAGTGCTGAAGGAAAACGGGCCTATGGCGTCGGACCCCCTATGCCTGACCTACTCCTACCTGTCCCA |
| | TGTGGACCTTGTGAAGGATCTCAACTCGGGACTGATCGGCGCCCTCTTGGTGTGCAGAGAAGGCAGCCTGGCGAAGG |
| | AAAAGACTCAGACCCTGCACAAGTTCATTCTGTTGTTTGCTGTGTTCGATGAAGGAAAGTCCTGGCACTCAGAAACC |
| | AAGAACTCGCTGATGCAGGATAGAGATGCGGCCTCGGCCAGAGCCTGGCCTAAAATGCACACCGTCAACGGATATGT |
| | GAACAGGTCGCTCCCTGGCCTCATCGGCTGCCACAGAAAGTCCGTGTATTGGCATGTGATCGGCATGGGTACTACTC |
| | CGGAAGTGCATAGTATCTTTCTGGAGGGCCATACCTTCTTGGTGCGCAACCACAGACAGGCCTCGCTGGAAATCTCG |
| | CCTATCACTTTCTTGACTGCGCAGACCCTCCTTATGGACCTTGGACAGTTCCTGCTGTTCTGTCACATCAGCTCCCA |
| | TCAGCATGATGGGATGGAGGCCTATGTCAAAGTGGACTCCTGCCCTGAGGAGCCACAGCTCCGGATGAAGAACAATG |
| | AGGAAGCGGAGGATTACGACGACGACCTGACTGACAGCGAAATGGACGTCGTGCGATTCGATGACGACAACAGCCCG |
| | TCCTTCATCCAAATTAGATCAGTGGCGAAGAAGCACCCCAAGACCTGGGTGCACTACATTGCCGCCGAGGAAGAGGA |
| | CTGGGACTACGCGCCGCTGGTGCTGGCGCCAGACGACAGGAGCTACAAGTCCCAGTACCTCAACAACGGGCCGCAGC |
| | GCATTGGCAGGAAGTACAAGAAAGTCCGCTTCATGGCCTACACTGATGAAACCTTCAAGACGAGGGAAGCCATCCAG |
| | CACGAGTCAGGCATCCTGGGACCGCTCCTTTACGGCGAAGTCGGGGATACCCTGCTCATCATTTTCAAGAACCAGGC |
| | ATCGCGGCCCTACAACATCTACCCTCACGGGATCACAGACGTGCGCCCGCTCTACTCCCGCCGGCTGCCCAAGGGAG |
| | TGAAGCACCTGAAGGATTTTCCCATCCTGCCGGGAGAAATCTTCAAGTACAAGTGGACCGTGACTGTGGAAGATGGC |
| | CCTACCAAGTCGGACCCTCGCTGTCTGACCCGGTACTATTCCTCGTTTGTGAACATGGAGCGCGACCTGGCCTCGGG |
| | GCTGATTGGTCCGCTGCTGATCTGCTACAAGGAGTCCGTGGACCAGCGCGGGAACCAGATCATGTCCGACAAGCGCA |
| | ACGTGATCCTGTTCTCTGTCTTTGATGAAAACAGATCGTGGTACTTGACTGAGAATATCCAGCGGTTCCTGCCCAAC |
| | CCAGCGGGAGTGCAACTGGAGGACCCGGAGTTCCAGGCCTCAAACATTATGCACTCTATCAACGGCTATGTGTTCGA |
| | CTCGCTCCAACTGAGCGTGTGCCTGCATGAAGTGGCATACTGGTACATTCTGTCCATCGGAGCCCAGACCGACTTCC |
| | TGTCCGTGTTCTTCTCCGGATACACCTTCAAGCATAAGATGGTGTACGAGGACACTCTGACCCTCTTCCCATTTTCC |
| | GGAGAAACTGTGTTCATGTCAATGGAAAACCCGGGCTTGTGGATTCTGGGTTGCCATAACTCGGACTTCCGGAATAG |
| | AGGGATGACCGCCCTGCTGAAAGTGTCCAGCTGTGACAAGAATACCGGCGATTACTACGAGGACAGCTATGAGGACA |
| | TCTCCGCTTATCTGCTGTCCAAGAACAACGCCATTGAACCCAGGTCCTTCTCCCAAAACGGTGCACCGACCTCCGAA |
| | AGCGCCACCCCAGAGTCAGGACCTGGCTCGGAACCGGCTACCTCGGGCTCAGAGACACCGGGGACTTCCGAGTCCGC |
| | AACCCCCGAGAGTGGACCCGGATCCGAACCAGCAACCTCAGGATCAGAAACCCCGGGAACTTCGGAATCCGCCACTC |
| | CCGAGTCGGGACCAGGCACCTCCACTGAGCCTTCCGAGGGAAGCGCCCCCGGATCCCCTGCTGGATCCCCTACCAGC |
| | ACTGAAGAAGGCACCTCAGAATCCGCGACCCCTGAGTCCGGCCCTGGAAGCGAACCCGCCACCTCCGGTTCCGAAAC |
| | CCCTGGGACTAGCGAGAGCGCCACTCCGGAATCGGGCCCAGGAAGCCCTGCCGGATCCCCGACCAGCACCGAGGAGG |
| | GAAGCCCCGCCGGGTCACCGACTTCCACTGAGGAGGGAGCCTCATCCCCCCCCGTGCTGAAGCGGCATCAAAGAGAG |
| | ATCACCAGGACCACTCTCCAGTCCGATCAGGAAGAAATTGACTACGACGATACTATCAGCGTGGAGATGAAGAAGGA |
| | GGACTTCGACATCTACGATGAGGATGAGAACCAGTCCCCTCGGAGCTTTCAGAAGAAAACCCGCCACTACTTCATCG |
| | CTGCCGTGGAGCGGCTGTGGGATTACGGGATGTCCAGCTCACCGCATGTGCTGCGGAATAGAGCGCAGTCAGGATCG |
| | GTGCCCCAGTTCAAGAAGGTCGTGTTCCAAGAGTTCACCGACGGGTCCTTCACTCAACCCCTGTACCGGGGCGAACT |
| | CAACGAACACCTGGGACTGCTTGGGCCGTATATCAGGGCAGAAGTGGAAGATAACATCATGGTCACCTTCCGCAACC |
| | AGGCCTCCCGGCCGTACAGCTTCTACTCTTCACTGATCTCCTACGAGGAAGATCAGCGGCAGGGAGCCGAGCCCCGG |
| | AAGAACTTCGTCAAGCCTAACGAAACTAAGACCTACTTTTGGAAGGTCCAGCATCACATGGCCCCGACCAAAGACGA |
| | GTTCGACTGTAAAGCCTGGGCCTACTTCTCCGATGTGGACCTGGAGAAGGACGTGCACTCGGGACTCATTGGCCCGC |
| | TCCTTGTGTGCCATACTAATACCCTGAACCCTGCTCACGGTCGCCAAGTCACAGTGCAGGAGTTCGCCCTCTTCTTC |
| | ACCATCTTCGATGAAACAAAGTCCTGGTACTTTACTGAGAACATGGAACGCAATTGCAGGGCACCCTGCAACATCCA |
| | GATGGAAGATCCCACCTTCAAGGAAAACTACCGGTTTCATGCCATTAACGGCTACATAATGGACACGTTGCCAGGAC |
| | TGGTCATGGCCCAGGACCAGAGAATCCGGTGGTATCTGCTCTCCATGGGCTCCAACGAAAACATTCACAGCATTCAT |
| | TTTTCCGGCCATGTGTTCACCGTCCGGAAGAAGGAAGAGTACAAGATGGCTCTGTACAACCTCTACCCTGGAGTGTT |
| | CGAGACTGTGGAAATGCTGCCTAGCAAGGCCGGCATTTGGAGAGTGGAATGCCTGATCGGAGAGCATTTGCACGCCG |
| | GAATGTCCACCCTGTTTCTTGTGTACTCCAACAAGTGCCAGACCCCGCTGGGAATGGCCTCAGGTCATATTAGGGAT |
| | TTCCAGATCACTGCTTCGGGGCAGTACGGGCAGTGGGCACCTAAGTTGGCCCGGCTGCACTACTCTGGCTCCATCAA |
| | TGCCTGGTCCACCAAGGAACCCTTCTCCTGGATTAAGGTGGACCTCCTGGCCCCAATGATTATTCACGGTATTAAGA |
| | CCCAGGGTGCCCGACAGAAGTTCTCCTCACTCTACATCTCGCAATTCATCATAATGTACAGCCTGGATGGGAAGAAG |
| | TGGCAGACCTACCGGGGAAACTCCACTGGAACGCTCATGGTGTTTTTCGGCAACGTGGACTCCTCCGGCATTAAGCA |
| | CAACATCTTCAACCCTCCGATCATTGCTCGGTACATCCGGCTGCACCCAACTCACTACAGCATCCGGTCCACCCTGC |
| | GGATGGAACTGATGGGTTGTGACCTGAACTCCTGCTCCATGCCCCTTGGGATGGAATCCAAGGCCATTAGCGATGCA |
| | CAGATCACCGCCTCTTCATACTTCACCAACATGTTCGCGACCTGGTCCCCGTCGAAGGCCCGCCTGCACCTCCAAGG |
| | TCGCTCCAATGCGTGGCGGCCTCAAGTGAACAACCCCAAGGAGTGGCTCCAGGTCGACTTCCAAAAGACCATGAAGG |
| | TCACCGGAGTGACCACCCAGGGCGTGAAGTCCCTGCTGACCTCTATGTACGTTAAGGAGTTCCTCATCTCCTCAAGC |
| | CAAGACGGACATCAGTGGACCCTGTTCTTCCAAAACGGAAAAGTCAAAGTATTCCAGGGCAACCAGGACTCCTTCAC |
| | CCCTGTGGTCAACAGCCTGGACCCCCCATTGCTGACCCGCTACCTCCGCATCCACCCCCAAAGCTGGGTCCACCAGA |
| | TCGCACTGCGCATGGAGGTCCTTGGATGCGAAGCCCAAGATCTGTACTAA |
| SEQ ID NO: 10 | ATRRYYLGAVELSWDYMQSDLGELPVDARFPPRVPKSFPFNTSVVYKKTLFVEFTDHLFNIAKPRPPWMGLLGPTIQ |
| Amino acid | AEVYDTVVITLKNMASHPVSLHAVGVSYWKASEGAEYDDQTSQREKEDDKVFPGGSHTYVWQVLKENGPMASDPLCL |
| sequence of | TYSYLSHVDLVKDLNSGLIGALLVCREGSLAKEKTQTLHKFILLFAVFDEGKSWHSETKNSLMQDRDAASARAWPKM |
| coBDDFVIIIX | HTVNGYVNRSLPGLIGCHRKSVYWHVIGMGTTPEVHSIFLEGHTFLVRNHRQASLEISPITFLTAQTLLMDLGQFLL |
| TEN (V2.0) | FCHISSHQHDGMEAYVKVDSCPEEPQLRMKNNEEAEDYDDDLTDSEMDVVRFDDDNSPSFIQIRSVAKKHPKTWVHY |
| | IAAEEEDWDYAPLVLAPDDRSYKSQYLNNGPQRIGRKYKKVRFMAYTDETFKTREAIQHESGILGPLLYGEVGDTLL |
| | IIFKNQASRPYNIYPHGITDVRPLYSRRLPKGVKHLKDFPILPGEIFKYKWTVTVEDGPTKSDPRCLTRYYSSFVNM |
| | ERDLASGLIGPLLICYKESVDQRGNQIMSDKRNVILFSVFDENRSWYLTENIQRFLPNPAGVQLEDPEFQASNIMHS |
| | INGYVFDSLQLSVCLHEVAYWYILSIGAQTDFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRSFSQNGAPTSESATPESGPGSEPATSGSET |
| | PGTSESATPESGPGSEPATSGSETPGTSESATPESGPGTSTEPSEGSAPGSPAGSPTSTEEGTSESATPESGPGSEP |
| | ATSGSETPGTSESATPESGPGSPAGSPTSTEEGSPAGSPTSTEEGASSPPVLKRHQREITRTTLQSDQEEIDYDDTI |
| | SVEMKKEDFDIYDEDENQSPRSFQKKTRHYFIAAVERLWDYGMSSSPHVLRNRAQSGSVPQFKKVVFQEFTDGSFTQ |
| | PLYRGELNEHLGLLGPYIRAEVEDNIMVTFRNQASRPYSFYSSLISYEEDQRQGAEPRKNFVKPNETKTYFWKVQHH |
| | MAPTKDEFDCKAWAYFSDVDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFSGHVFTVRKKEEYKMALY |
| | NLYPGVFETVEMLPSKAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRGNSTGTLMVFFGNV |
| | DSSGIKHNIFNPPIIARYIRLHPTHYSIRSTLRMELMGCDLNSCSMPLGMESKAISDAQITASSYFTNMFATWSPSK |
| | ARLHLQGRSNAWRPQVNNPKEWLQVDFQKTMKVTGVTTQGVKSLLTSMYVKEFLISSSQDGHQWTLFFQNGKVKVFQ |
| | GNQDSFTPVVNSLDPPLLTRYLRIHPQSWVHQIALRMEVLGCEAQDLY |
| SEQ ID NO: 11 | MQIELSTCFFLCLLRFCFS |
| Signal | |
| peptide of | |
| coBDDFVIIIX | |
| TEN (V2.0) | |
| SEQ ID NO: 12 | ATRRYYLGAVELSWDYMQSDLGELPVDARFPPRVPKSFPFNTSVVYKKTLFVEFTDHLFNIAKPRPPWMGLLGPTIQ |
| Amino acid | AEVYDTVVITLKNMASHPVSLHAVGVSYWKASEGAEYDDQTSQREKEDDKVFPGGSHTYVWQVLKENGPMASDPLCL |
| sequence of | TYSYLSHVDLVKDLNSGLIGALLVCREGSLAKEKTQTLHKFILLFAVFDEGKSWHSETKNSLMQDRDAASARAWPKM |
| BDD mature | HTVNGYVNRSLPGLIGCHRKSVYWHVIGMGTTPEVHSIFLEGHTFLVRNHRQASLEISPITFLTAQTLLMDLGQFLL |
| human FVIII | FCHISSHQHDGMEAYVKVDSCPEEPQLRMKNNEEAEDYDDDLTDSEMDVVRFDDDNSPSFIQIRSVAKKHPKTWVHY |
| | IAAEEEDWDYAPLVLAPDDRSYKSQYLNNGPQRIGRKYKKVRFMAYTDETFKTREAIQHESGILGPLLYGEVGDTLL |
| | IIFKNQASRPYNIYPHGITDVRPLYSRRLPKGVKHLKDFPILPGEIFKYKWTVTVEDGPTKSDPRCLTRYYSSFVNM |
| | ERDLASGLIGPLLICYKESVDQRGNQIMSDKRNVILFSVFDENRSWYLTENIQRFLPNPAGVQLEDPEFQASNIMHS |
| | INGYVFDSLQLSVCLHEVAYWYILSIGAQTDFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRSFSQNPPVLKRHQREITRTTLQSDQEEIDY |
| | DDTISVEMKKEDFDIYDEDENQSPRSFQKKTRHYFIAAVERLWDYGMSSSPHVLRNRAQSGSVPQFKKVVFQEFTDG |
| | SFTQPLYRGELNEHLGLLGPYIRAEVEDNIMVTFRNQASRPYSFYSSLISYEEDQRQGAEPRKNFVKPNETKTYFWK |
| | VQHHMAPTKDEFDCKAWAYFSDVDLEKDVHSGLIGPLLVCHTNTLNPAHGRQVTVQEFALFFTIFDETKSWYFTENM |
| | ERNCRAPCNIQMEDPTFKENYRFHAINGYIMDTLPGLVMAQDQRIRWYLLSMGSNENIHSIHFSGHVFTVRKKEEYK |
| | MALYNLYPGVFETVEMLPSKAGIWRVECLIGEHLHAGMSTLFLVYSNKCQTPLGMASGHIRDFQITASGQYGQWAPK |
| | LARLHYSGSINAWSTKEPFSWIKVDLLAPMIIHGIKTQGARQKFSSLYISQFIIMYSLDGKKWQTYRGNSTGTLMVF |
| | FGNVDSSGIKHNIFNPPIIARYIRLHPTHYSIRSTLRMELMGCDLNSCSMPLGMESKAISDAQITASSYFTNMFATW |
| | SPSKARLHLQGRSNAWRPQVNNPKEWLQVDFQKTMKVTGVTTQGVKSLLTSMYVKEFLISSSQDGHQWTLFFQNGKV |
| | KVFQGNQDSFTPVVNSLDPPLLTRYLRIHPQSWVHQIALRMEVLGCEAQDLY |
| SEQ ID NO: 13 | ATGCAAATAGAGCTCTCCACCTGCTTCTTTCTGTGCCTTTTGCGATTCTGCTTTAGTGCCACCAGAAGATACTACCT |
| Nucleotide | GGGTGCAGTGGAACTGTCATGGGACTATATGCAAAGTGATCTCGGTGAGCTGCCTGTGGACGCAAGATTTCCTCCTA |
| sequence | GAGTGCCAAAATCTTTTCCATTCAACACCTCAGTCGTGTACAAAAAGACTCTGTTTGTAGAATTCACGGATCACCTT |
| encoding | TTCAACATCGCTAAGCCAAGGCCACCCTGGATGGGTCTGCTAGGTCCTACCATCCAGGCTGAGGTTTATGATACAGT |
| BDD mature | GGTCATTACACTTAAGAACATGGCTTCCCATCCTGTCAGTCTTCATGCTGTTGGTGTATCCTACTGGAAAGCTTCTG |
| human FVIII | AGGGAGCTGAATATGATGATCAGACCAGTCAAAGGGAGAAAGAAGATGATAAAGTCTTCCCTGGTGGAAGCCATACA |
| | TATGTCTGGCAGGTCCTGAAAGAGAATGGTCCAATGGCCTCTGACCCACTGTGCCTTACCTACTCATATCTTTCTCA |
| | TGTGGACCTGGTAAAAGACTTGAATTCAGGCCTCATTGGAGCCCTACTAGTATGTAGAGAAGGGAGTCTGGCCAAGG |
| | AAAAGACACAGACCTTGCACAAATTTATACTACTTTTTGCTGTATTTGATGAAGGGAAAAGTTGGCACTCAGAAACA |
| | AAGAACTCCTTGATGCAGGATAGGGATGCTGCATCTGCTCGGGCCTGGCCTAAAATGCACACAGTCAATGGTTATGT |
| | AAACAGGTCTCTGCCAGGTCTGATTGGATGCCACAGGAAATCAGTCTATTGGCATGTGATTGGAATGGGCACCACTC |
| | CTGAAGTGCACTCAATATTCCTCGAAGGTCACACATTTCTTGTGAGGAACCATCGCCAGGCGTCCTTGGAAATCTCG |
| | CCAATAACTTTCCTTACTGCTCAAACACTCTTGATGGACCTTGGACAGTTTCTACTGTTTTGTCATATCTCTTCCCA |
| | CCAACATGATGGCATGGAAGCTTATGTCAAAGTAGACAGCTGTCCAGAGGAACCCCAACTACGAATGAAAAATAATG |
| | AAGAAGCGGAAGACTATGATGATGATCTTACTGATTCTGAAATGGATGTGGTCAGGTTTGATGATGACAACTCTCCT |
| | TCCTTTATCCAAATTCGCTCAGTTGCCAAGAAGCATCCTAAAACTTGGGTACATTACATTGCTGCTGAAGAGGAGGA |
| | CTGGGACTATGCTCCCTTAGTCCTCGCCCCCGATGACAGAAGTTATAAAAGTCAATATTTGAACAATGGCCCTCAGC |
| | GGATTGGTAGGAAGTACAAAAAAGTCCGATTTATGGCATACACAGATGAAACCTTTAAGACTCGTGAAGCTATTCAG |
| | CATGAATCAGGAATCTTGGGACCTTTACTTTATGGGGAAGTTGGAGACACACTGTTGATTATATTTAAGAATCAAGC |
| | AAGCAGACCATATAACATCTACCCTCACGGAATCACTGATGTCCGTCCTTTGTATTCAAGGAGATTACCAAAAGGTG |
| | TAAAACATTTGAAGGATTTTCCAATTCTGCCAGGAGAAATATTCAAATATAAATGGACAGTGACTGTAGAAGATGGG |
| | CCAACTAAATCAGATCCTCGGTGCCTGACCCGCTATTACTCTAGTTTCGTTAATATGGAGAGAGATCTAGCTTCAGG |
| | ACTCATTGGCCCTCTCCTCATCTGCTACAAAGAATCTGTAGATCAAAGAGGAAACCAGATAATGTCAGACAAGAGGA |
| | ATGTCATCCTGTTTTCTGTATTTGATGAGAACCGAAGCTGGTACCTCACAGAGAATATACAACGCTTTCTCCCCAAT |
| | CCAGCTGGAGTGCAGCTTGAGGATCCAGAGTTCCAAGCCTCCAACATCATGCACAGCATCAATGGCTATGTTTTTGA |
| | TAGTTTGCAGTTGTCAGTTTGTTTGCATGAGGTGGCATACTGGTACATTCTAAGCATTGGAGCACAGACTGACTTCC |
| | TTTCTGTCTTCTTCTCTGGATATACCTTCAAACACAAAATGGTCTATGAAGACACACTCACCCTATTCCCATTCTCA |
| | GGAGAAACTGTCTTCATGTCGATGGAAAACCCAGGTCTATGGATTCTGGGGTGCCACAACTCAGACTTTCGGAACAG |
| | AGGCATGACCGCCTTACTGAAGGTTTCTAGTTGTGACAAGAACACTGGTGATTATTACGAGGACAGTTATGAAGATA |
| | TTTCAGCATACTTGCTGAGTAAAAACAATGCCATTGAACCAAGAAGCTTCTCTCAAAACCCACCAGTCTTGAAACGC |
| | CATCAACGGGAAATAACTCGTACTACTCTTCAGTCAGATCAAGAGGAAATTGACTATGATGATACCATATCAGTTGA |
| | AATGAAGAAGGAAGATTTTGACATTTATGATGAGGATGAAAATCAGAGCCCCCGCAGCTTTCAAAAGAAAACACGAC |
| | ACTATTTTATTGCTGCAGTGGAGAGGCTCTGGGATTATGGGATGAGTAGCTCCCCACATGTTCTAAGAAACAGGGCT |
| | CAGAGTGGCAGTGTCCCTCAGTTCAAGAAAGTTGTTTTCCAGGAATTTACTGATGGCTCCTTTACTCAGCCCTTATA |
| | CCGTGGAGAACTAAATGAACATTTGGGACTCCTGGGGCCATATATAAGAGCAGAAGTTGAAGATAATATCATGGTAA |
| | CTTTCAGAAATCAGGCCTCTCGTCCCTATTCCTTCTATTCTAGCCTTATTTCTTATGAGGAAGATCAGAGGCAAGGA |
| | GCAGAACCTAGAAAAAACTTTGTCAAGCCTAATGAAACCAAAACTTACTTTTGGAAAGTGCAACATCATATGGCACC |
| | CACTAAAGATGAGTTTGACTGCAAAGCCTGGGCTTATTTCTCTGATGTTGACCTGGAAAAAGATGTGCACTCAGGCC |
| | TGATTGGACCCCTTCTGGTCTGCCACACTAACACACTGAACCCTGCTCATGGGAGACAAGTGACAGTACAGGAATTT |
| | GCTCTGTTTTTCACCATCTTTGATGAGACCAAAAGCTGGTACTTCACTGAAAATATGGAAAGAAACTGCAGGGCTCC |
| | CTGCAATATCCAGATGGAAGATCCCACTTTTAAAGAGAATTATCGCTTCCATGCAATCAATGGCTACATAATGGATA |
| | CACTACCTGGCTTAGTAATGGCTCAGGATCAAAGGATTCGATGGTATCTGCTCAGCATGGGCAGCAATGAAAACATC |
| | CATTCTATTCATTTCAGTGGACATGTGTTCACTGTACGAAAAAAAGAGGAGTATAAAATGGCACTGTACAATCTCTA |
| | TCCAGGTGTTTTTGAGACAGTGGAAATGTTACCATCCAAAGCTGGAATTTGGCGGGTGGAATGCCTTATTGGCGAGC |
| | ATCTACATGCTGGGATGAGCACACTTTTTCTGGTGTACAGCAATAAGTGTCAGACTCCCCTGGGAATGGCTTCTGGA |
| | CACATTAGAGATTTTCAGATTACAGCTTCAGGACAATATGGACAGTGGGCCCCAAAGCTGGCCAGACTTCATTATTC |
| | CGGATCAATCAATGCCTGGAGCACCAAGGAGCCCTTTTCTTGGATCAAGGTGGATCTGTTGGCACCAATGATTATTC |
| | ACGGCATCAAGACCCAGGGTGCCCGTCAGAAGTTCTCCAGCCTCTACATCTCTCAGTTTATCATCATGTATAGTCTT |
| | GATGGGAAGAAGTGGCAGACTTATCGAGGAAATTCCACTGGAACCTTAATGGTCTTCTTTGGCAATGTGGATTCATC |
| | TGGGATAAAACACAATATTTTTAACCCTCCAATTATTGCTCGATACATCCGTTTGCACCCAACTCATTATAGCATTC |
| | GCAGCACTCTTCGCATGGAGTTGATGGGCTGTGATTTAAATAGTTGCAGCATGCCATTGGGAATGGAGAGTAAAGCA |
| | ATATCAGATGCACAGATTACTGCTTCATCCTACTTTACCAATATGTTTGCCACCTGGTCTCCTTCAAAAGCTCGACT |
| | TCACCTCCAAGGGAGGAGTAATGCCTGGAGACCTCAGGTGAATAATCCAAAAGAGTGGCTGCAAGTGGACTTCCAGA |
| | AGACAATGAAAGTCACAGGAGTAACTACTCAGGGAGTAAAATCTCTGCTTACCAGCATGTATGTGAAGGAGTTCCTC |
| | ATCTCCAGCAGTCAAGATGGCCATCAGTGGACTCTCTTTTTTCAGAATGGCAAAGTAAAGGTTTTTCAGGGAAATCA |
| | AGACTCCTTCACACCTGTGGTGAACTCTCTAGACCCACCGTTACTGACTCGCTACCTTCGAATTCACCCCCAGAGTT |
| | GGGTGCACCAGATTGCCCTGAGGATGGAGGTTCTGGGCTGCGAGGCACAGGACCTCTAC |
| SEQ ID NO: 14 | GGCCCCAGGTTAATTTTTAAAAAGCAGTCAAAGGTCAAAGTGGCCCTTGGCAGCATTTACTCTCTCTATTGACTTTG |
| V2.0 | GTTAATAATCTCAGGAGCACAAACATTCCTGGAGGCAGGAGAAGAAATCAACATCCTGGACTTATCCTCTGGGCCTC |
| Expression | TCCCCACCTTCGATGGCCCCAGGTTAATTTTTAAAAAGCAGTCAAAGGTCAAAGTGGCCCTTGGCAGCATTTACTCT |
| cassette | CTCTATTGACTTTGGTTAATAATCTCAGGAGCACAAACATTCCTGGAGGCAGGAGAAGAAATCAACATCCTGGACTT |
| mTTR482- | ATCCTCTGGGCCTCTCCCCACCGATATCTACCTGCTGATCGCCCGGCCCCTGTTCAAACATGTCCTAATACTCTGTC |
| Intron- | GGGGCAAAGGTCGGCAGTAGTTTTCCATCTTACTCAACATCCTCCCAGTGTACGTAGGATCCTGTCTGTCTGCACAT |
| coBDDFVIIIX | TTCGTAGAGCGAGTGTTCCGATACTCTAATCTCCCGGGGCAAAGGTCGTATTGACTTAGGTTACTTATTCTCCTTTT |
| TEN (V2.0)- | GTTGACTAAGTCAATAATCAGAATCAGCAGGTTTGGAGTCAGCTTGGCAGGGATCAGCAGCCTGGGTTGGAAGGAGG |
| WERE- | GGGTATAAAAGCCCCTTCACCAGGAGAAGCCGTCACACAGATCCACAAGCTCCTGCTAGGAATTCTCAGGAGCACAA |
| bGHPolyA | ACATTCCTGGAGGCAGGAGAAGAAATCAACATCCTGGACTTATCCTCTGGGCCTCTCCCCACCGATATCTACCTGCT |
| | GATCGCCCGGCCCCTGTTCAAACATGTCCTAATACTCTGTCGGGGCAAAGGTCGGCAGTAGTTTTCCATCTTACTCA |
| | ACATCCTCCCAGTGTACGTAGGATCCTGTCTGTCTGCACATTTCGTAGAGCGAGTGTTCCGATACTCTAATCTCCCG |
| | GGGCAAAGGTCGTATTGACTTAGGTTACTTATTCTCCTTTTGTTGACTAAGTCAATAATCAGAATCAGCAGGTTTGG |
| | AGTCAGCTTGGCAGGGATCAGCAGCCTGGGTTGGAAGGAGGGGGTATAAAAGCCCCTTCACCAGGAGAAGCCGTCAC |
| | ACAGATCCACAAGCTCCTGCTAGAGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGGCCCTTCTCCTCCGGGCTGTAATT |
| | AGCGCTTGGTTTATTGACGGCTTGTTTCTTTTCTGTGGCTGCGTGAAAGCCTTGAGGGGCTCCGGGAAGGCCCTTTG |
| | TGCGGGGGGAGCGGCTCGGGGGGTGCGTGCGTGTGTGTGTGCGTGGGGAGCGCCGCGTGCGGCTCCGCGCTGCCCGG |
| | CGGCTGTGAGCGCTGCGGGCGCGGCGCGGGGCTTTGTGCGCTCCGCAGTGTGCGCGAGGGGAGCGCGGCCGGGGGCG |
| | GTGCCCCGCGGTGCGGGGGGGGCTGCGAGGGGAACAAAGGCTGCGTGCGGGGTGTGTGCGTGGGGGGGTGAGCAGGG |
| | GGTGTGGGCGCGTCGGTCGGGCTGCAACCCCCCCTGCACCCCCCTCCCCGAGTTGCTGAGCACGGCCCGGCTTCGGG |
| | TGCGGGGCTCCGTACGGGGCGTGGCGCGGGGCTCGCCGTGCCGGGCGGGGGGTGGCGGCAGGTGGGGGTGCCGGGCG |
| | GGGCGGGGCCGCCTCGGGCCGGGGAGGGCTCGGGGGAGGGGCGCGGCGGCCCCCGGAGCGCCGGCGGCTGTCGAGGC |
| | GCGGCGAGCCGCAGCCATTGCCTTTTATGGTAATCGTGCGAGAGGGCGCAGGGACTTCCTTTGTCCCAAATCTGTGC |
| | GGAGCCGAAATCTGGGAGGCGCCGCCGCACCCCCTCTAGCGGGCGCGGGGCGAAGCGGTGCGGCGCCGGCAGGAAGG |
| | AAATGGGCGGGGAGGGCCTTCGTGCGTCGCCGCGCCGCCGTCCCCTTCTCCCTCTCCAGCCTCGGGGCTGTCCGCGG |
| | GGGGACGGCTGCCTTCGGGGGGGACGGGGCAGGGCGGGGTTCGGCTTCTGGCGTGTGACCGGCGGCTCTAGAGCCTC |
| | TGCTAACCTTGTTCTTGCCTTCTTCTTTTTCCTACAGCTCCTGGGCAACGTGCTGGTTATTGTGCTGTCTCATCATT |
| | TTGGCAAAGAATTACTCGAGGCCACCATGCAGATTGAACTGTCCACTTGCTTCTTCCTGTGCCTCCTGCGGTTTTGC |
| | TTCTCGGCCACCCGCCGGTATTACTTAGGTGCTGTGGAACTGAGCTGGGACTACATGCAGTCCGACCTGGGAGAACT |
| | GCCGGTGGACGCGAGATTCCCACCTAGAGTCCCGAAGTCCTTCCCATTCAACACCTCCGTGGTCTACAAAAAGACCC |
| | TGTTCGTGGAGTTCACTGACCACCTTTTCAATATTGCCAAGCCGCGCCCCCCCTGGATGGGCCTGCTTGGTCCTACG |
| | ATCCAAGCAGAGGTCTACGACACCGTGGTCATCACACTGAAGAACATGGCCTCACACCCCGTGTCGCTGCATGCTGT |
| | GGGAGTGTCCTACTGGAAGGCCTCAGAGGGTGCCGAATATGATGACCAGACCAGCCAGAGGGAAAAGGAGGATGACA |
| | AAGTGTTCCCGGGTGGCAGCCACACTTACGTGTGGCAAGTGCTGAAGGAAAACGGGCCTATGGCGTCGGACCCCCTA |
| | TGCCTGACCTACTCCTACCTGTCCCATGTGGACCTTGTGAAGGATCTCAACTCGGGACTGATCGGCGCCCTCTTGGT |
| | GTGCAGAGAAGGCAGCCTGGCGAAGGAAAAGACTCAGACCCTGCACAAGTTCATTCTGTTGTTTGCTGTGTTCGATG |
| | AAGGAAAGTCCTGGCACTCAGAAACCAAGAACTCGCTGATGCAGGATAGAGATGCGGCCTCGGCCAGAGCCTGGCCT |
| | AAAATGCACACCGTCAACGGATATGTGAACAGGTCGCTCCCTGGCCTCATCGGCTGCCACAGAAAGTCCGTGTATTG |
| | GCATGTGATCGGCATGGGTACTACTCCGGAAGTGCATAGTATCTTTCTGGAGGGCCATACCTTCTTGGTGCGCAACC |
| | ACAGACAGGCCTCGCTGGAAATCTCGCCTATCACTTTCTTGACTGCGCAGACCCTCCTTATGGACCTTGGACAGTTC |
| | CTGCTGTTCTGTCACATCAGCTCCCATCAGCATGATGGGATGGAGGCCTATGTCAAAGTGGACTCCTGCCCTGAGGA |
| | GCCACAGCTCCGGATGAAGAACAATGAGGAAGCGGAGGATTACGACGACGACCTGACTGACAGCGAAATGGACGTCG |
| | TGCGATTCGATGACGACAACAGCCCGTCCTTCATCCAAATTAGATCAGTGGCGAAGAAGCACCCCAAGACCTGGGTG |
| | CACTACATTGCCGCCGAGGAAGAGGACTGGGACTACGCGCCGCTGGTGCTGGCGCCAGACGACAGGAGCTACAAGTC |
| | CCAGTACCTCAACAACGGGCCGCAGCGCATTGGCAGGAAGTACAAGAAAGTCCGCTTCATGGCCTACACTGATGAAA |
| | CCTTCAAGACGAGGGAAGCCATCCAGCACGAGTCAGGCATCCTGGGACCGCTCCTTTACGGCGAAGTCGGGGATACC |
| | CTGCTCATCATTTTCAAGAACCAGGCATCGCGGCCCTACAACATCTACCCTCACGGGATCACAGACGTGCGCCCGCT |
| | CTACTCCCGCCGGCTGCCCAAGGGAGTGAAGCACCTGAAGGATTTTCCCATCCTGCCGGGAGAAATCTTCAAGTACA |
| | AGTGGACCGTGACTGTGGAAGATGGCCCTACCAAGTCGGACCCTCGCTGTCTGACCCGGTACTATTCCTCGTTTGTG |
| | AACATGGAGCGCGACCTGGCCTCGGGGCTGATTGGTCCGCTGCTGATCTGCTACAAGGAGTCCGTGGACCAGCGCGG |
| | GAACCAGATCATGTCCGACAAGCGCAACGTGATCCTGTTCTCTGTCTTTGATGAAAACAGATCGTGGTACTTGACTG |
| | AGAATATCCAGCGGTTCCTGCCCAACCCAGCGGGAGTGCAACTGGAGGACCCGGAGTTCCAGGCCTCAAACATTATG |
| | CACTCTATCAACGGCTATGTGTTCGACTCGCTCCAACTGAGCGTGTGCCTGCATGAAGTGGCATACTGGTACATTCT |
| | GTCCATCGGAGCCCAGACCGACTTCCTGTCCGTGTTCTTCTCCGGATACACCTTCAAGCATAAGATGGTGTACGAGG |
| | ACACTCTGACCCTCTTCCCATTTTCCGGAGAAACTGTGTTCATGTCAATGGAAAACCCGGGCTTGTGGATTCTGGGT |
| | TGCCATAACTCGGACTTCCGGAATAGAGGGATGACCGCCCTGCTGAAAGTGTCCAGCTGTGACAAGAATACCGGCGA |
| | TTACTACGAGGACAGCTATGAGGACATCTCCGCTTATCTGCTGTCCAAGAACAACGCCATTGAACCCAGGTCCTTCT |
| | CCCAAAACGGTGCACCGACCTCCGAAAGCGCCACCCCAGAGTCAGGACCTGGCTCGGAACCGGCTACCTCGGGCTCA |
| | GAGACACCGGGGACTTCCGAGTCCGCAACCCCCGAGAGTGGACCCGGATCCGAACCAGCAACCTCAGGATCAGAAAC |
| | CCCGGGAACTTCGGAATCCGCCACTCCCGAGTCGGGACCAGGCACCTCCACTGAGCCTTCCGAGGGAAGCGCCCCCG |
| | GATCCCCTGCTGGATCCCCTACCAGCACTGAAGAAGGCACCTCAGAATCCGCGACCCCTGAGTCCGGCCCTGGAAGC |
| | GAACCCGCCACCTCCGGTTCCGAAACCCCTGGGACTAGCGAGAGCGCCACTCCGGAATCGGGCCCAGGAAGCCCTGC |
| | CGGATCCCCGACCAGCACCGAGGAGGGAAGCCCCGCCGGGTCACCGACTTCCACTGAGGAGGGAGCCTCATCCCCCC |
| | CCGTGCTGAAGCGGCATCAAAGAGAGATCACCAGGACCACTCTCCAGTCCGATCAGGAAGAAATTGACTACGACGAT |
| | ACTATCAGCGTGGAGATGAAGAAGGAGGACTTCGACATCTACGATGAGGATGAGAACCAGTCCCCTCGGAGCTTTCA |
| | GAAGAAAACCCGCCACTACTTCATCGCTGCCGTGGAGCGGCTGTGGGATTACGGGATGTCCAGCTCACCGCATGTGC |
| | TGCGGAATAGAGCGCAGTCAGGATCGGTGCCCCAGTTCAAGAAGGTCGTGTTCCAAGAGTTCACCGACGGGTCCTTC |
| | ACTCAACCCCTGTACCGGGGCGAACTCAACGAACACCTGGGACTGCTTGGGCCGTATATCAGGGCAGAAGTGGAAGA |
| | TAACATCATGGTCACCTTCCGCAACCAGGCCTCCCGGCCGTACAGCTTCTACTCTTCACTGATCTCCTACGAGGAAG |
| | ATCAGCGGCAGGGAGCCGAGCCCCGGAAGAACTTCGTCAAGCCTAACGAAACTAAGACCTACTTTTGGAAGGTCCAG |
| | CATCACATGGCCCCGACCAAAGACGAGTTCGACTGTAAAGCCTGGGCCTACTTCTCCGATGTGGACCTGGAGAAGGA |
| | CGTGCACTCGGGACTCATTGGCCCGCTCCTTGTGTGCCATACTAATACCCTGAACCCTGCTCACGGTCGCCAAGTCA |
| | CAGTGCAGGAGTTCGCCCTCTTCTTCACCATCTTCGATGAAACAAAGTCCTGGTACTTTACTGAGAACATGGAACGC |
| | AATTGCAGGGCACCCTGCAACATCCAGATGGAAGATCCCACCTTCAAGGAAAACTACCGGTTTCATGCCATTAACGG |
| | CTACATAATGGACACGTTGCCAGGACTGGTCATGGCCCAGGACCAGAGAATCCGGTGGTATCTGCTCTCCATGGGCT |
| | CCAACGAAAACATTCACAGCATTCATTTTTCCGGCCATGTGTTCACCGTCCGGAAGAAGGAAGAGTACAAGATGGCT |
| | CTGTACAACCTCTACCCTGGAGTGTTCGAGACTGTGGAAATGCTGCCTAGCAAGGCCGGCATTTGGAGAGTGGAATG |
| | CCTGATCGGAGAGCATTTGCACGCCGGAATGTCCACCCTGTTTCTTGTGTACTCCAACAAGTGCCAGACCCCGCTGG |
| | GAATGGCCTCAGGTCATATTAGGGATTTCCAGATCACTGCTTCGGGGCAGTACGGGCAGTGGGCACCTAAGTTGGCC |
| | CGGCTGCACTACTCTGGCTCCATCAATGCCTGGTCCACCAAGGAACCCTTCTCCTGGATTAAGGTGGACCTCCTGGC |
| | CCCAATGATTATTCACGGTATTAAGACCCAGGGTGCCCGACAGAAGTTCTCCTCACTCTACATCTCGCAATTCATCA |
| | TAATGTACAGCCTGGATGGGAAGAAGTGGCAGACCTACCGGGGAAACTCCACTGGAACGCTCATGGTGTTTTTCGGC |
| | AACGTGGACTCCTCCGGCATTAAGCACAACATCTTCAACCCTCCGATCATTGCTCGGTACATCCGGCTGCACCCAAC |
| | TCACTACAGCATCCGGTCCACCCTGCGGATGGAACTGATGGGTTGTGACCTGAACTCCTGCTCCATGCCCCTTGGGA |
| | TGGAATCCAAGGCCATTAGCGATGCACAGATCACCGCCTCTTCATACTTCACCAACATGTTCGCGACCTGGTCCCCG |
| | TCGAAGGCCCGCCTGCACCTCCAAGGTCGCTCCAATGCGTGGCGGCCTCAAGTGAACAACCCCAAGGAGTGGCTCCA |
| | GGTCGACTTCCAAAAGACCATGAAGGTCACCGGAGTGACCACCCAGGGCGTGAAGTCCCTGCTGACCTCTATGTACG |
| | TTAAGGAGTTCCTCATCTCCTCAAGCCAAGACGGACATCAGTGGACCCTGTTCTTCCAAAACGGAAAAGTCAAAGTA |
| | TTCCAGGGCAACCAGGACTCCTTCACCCCTGTGGTCAACAGCCTGGACCCCCCATTGCTGACCCGCTACCTCCGCAT |
| | CCACCCCCAAAGCTGGGTCCACCAGATCGCACTGCGCATGGAGGTCCTTGGATGCGAAGCCCAAGATCTGTACTAAG |
| | CGGCCGCTCATAATCAACCTCTGGATTACAAAATTTGTGAAAGATTGACTGGTATTCTTAACTATGTTGCTCCTTTT |
| | ACGCTATGTGGATACGCTGCTTTAATGCCTTTGTATCATGCTATTGCTTCCCGTATGGCTTTCATTTTCTCCTCCTT |
| | GTATAAATCCTGGTTGCTGTCTCTTTATGAGGAGTTGTGGCCCGTTGTCAGGCAACGTGGCGTGGTGTGCACTGTGT |
| | TTGCTGACGCAACCCCCACTGGTTGGGGCATTGCCACCACCTGTCAGCTCCTTTCCGGGACTTTCGCTTTCCCCCTC |
| | CCTATTGCCACGGCGGAACTCATCGCCGCCTGCCTTGCCCGCTGCTGGACAGGGGCTCGGCTGTTGGGCACTGACAA |
| | TTCCGTGGTGTTGTCGGGGAAATCATCGTCCTTTCCTTGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCT |
| | CTTCCGCGTCTTCGCCTTCGCCCTCAGACGAGTCGGATCTCCCTTTGGGCCGCCTCCCCGCTGCCTAGGCGACTGTG |
| | CCTTCTAGTTGCCAGCCATCTGTTGTTTGCCCCTCCCCCGTGCCTTCCTTGACCCTGGAAGGTGCCACTCCCACTGT |
| | CCTTTCCTAATAAAATGAGGAAATTGCATCGCATTGTCTGAGTAGGTGTCATTCTATTCTGGGGGGTGGGGTGGGGC |
| | AGGACAGCAAGGGGGAGGATTGGGAAGACAATAGCAGGCATGCTGGGGAAGACCATGGGCGCGCCAGGCCTGTCGAC |
| | GCCCGGGCGGTACCGCGATCGCTCGCGACGCATAAAG |
| SEQ ID NO: 15 | GGCCCCAGGTTAATTTTTAAAAAGCAGTCAAAGGTCAAAGTGGCCCTTGGCAGCATTTACTCTCTCTATTGACTTTG |
| A1MB2 | GTTAATAATCTCAGGAGCACAAACATTCCTGGAGGCAGGAGAAGAAATCAACATCCTGGACTTATCCTCTGGGCCTC |
| enhancer | TCCCCACCTTCGATGGCCCCAGGTTAATTTTTAAAAAGCAGTCAAAGGTCAAAGTGGCCCTTGGCAGCATTTACTCT |
| | CTCTATTGACTTTGGTTAATAATCTCAGGAGCACAAACATTCCTGGAGGCAGGAGAAGAAATCAACATCCTGGACTT |
| | ATCCTCTGGGCCTCTCCCCACC |
| SEQ ID NO: 16 | GATATCTACCTGCTGATCGCCCGGCCCCTGTTCAAACATGTCCTAATACTCTGTCGGGGCAAAGGTCGGCAGTAGTT |
| mTTR | TTCCATCTTACTCAACATCCTCCCAGTGTACGTAGGATCCTGTCTGTCTGCACATTTCGTAGAGCGAGTGTTCCGAT |
| promoter | ACTCTAATCTCCCGGGGCAAAGGTCGTATTGACTTAGGTTACTTATTCTCCTTTTGTTGACTAAGTCAATAATCAGA |
| | ATCAGCAGGTTTGGAGTCAGCTTGGCAGGGATCAGCAGCCTGGGTTGGAAGGAGGGGGTATAAAAGCCCCTTCACCA |
| | GGAGAAGCCGTCACACAGATCCACAAGCTCCTGCTAG |
| SEQ ID NO: 17 | TCAGGAGCACAAACATTCCTGGAGGCAGGAGAAGAAATCAACATCCTGGACTTATCCTCTGGGCCTCTCCCCACCGA |
| Chimeric | TATCTACCTGCTGATCGCCCGGCCCCTGTTCAAACATGTCCTAATACTCTGTCGGGGCAAAGGTCGGCAGTAGTTTT |
| Intron | CCATCTTACTCAACATCCTCCCAGTGTACGTAGGATCCTGTCTGTCTGCACATTTCGTAGAGCGAGTGTTCCGATAC |
| | TCTAATCTCCCGGGGCAAAGGTCGTATTGACTTAGGTTACTTATTCTCCTTTTGTTGACTAAGTCAATAATCAGAAT |
| | CAGCAGGTTTGGAGTCAGCTTGGCAGGGATCAGCAGCCTGGGTTGGAAGGAGGGGGTATAAAAGCCCCTTCACCAGG |
| | AGAAGCCGTCACACAGATCCACAAGCTCCTGCTAGAGTCGCTGCGCGCTGCCTTCGCCCCGTGCCCCGCTCCGCCGC |
| | CGCCTCGCGCCGCCCGCCCCGGCTCTGACTGACCGCGTTACTCCCACAGGTGAGCGGGCGGGACGGCCCTTCTCCTC |
| | CGGGCTGTAATTAGCGCTTGGTTTATTGACGGCTTGTTTCTTTTCTGTGGCTGCGTGAAAGCCTTGAGGGGCTCCGG |
| | GAAGGCCCTTTGTGCGGGGGGAGCGGCTCGGGGGGTGCGTGCGTGTGTGTGTGCGTGGGGAGCGCCGCGTGCGGCTC |
| | CGCGCTGCCCGGCGGCTGTGAGCGCTGCGGGCGCGGCGCGGGGCTTTGTGCGCTCCGCAGTGTGCGCGAGGGGAGCG |
| | CGGCCGGGGGCGGTGCCCCGCGGTGCGGGGGGGGCTGCGAGGGGAACAAAGGCTGCGTGCGGGGTGTGTGCGTGGGG |
| | GGGTGAGCAGGGGGTGTGGGCGCGTCGGTCGGGCTGCAACCCCCCCTGCACCCCCCTCCCCGAGTTGCTGAGCACGG |
| | CCCGGCTTCGGGTGCGGGGCTCCGTACGGGGCGTGGCGCGGGGCTCGCCGTGCCGGGCGGGGGGTGGCGGCAGGTGG |
| | GGGTGCCGGGCGGGGCGGGGCCGCCTCGGGCCGGGGAGGGCTCGGGGGAGGGGCGCGGCGGCCCCCGGAGCGCCGGC |
| | GGCTGTCGAGGCGCGGCGAGCCGCAGCCATTGCCTTTTATGGTAATCGTGCGAGAGGGCGCAGGGACTTCCTTTGTC |
| | CCAAATCTGTGCGGAGCCGAAATCTGGGAGGCGCCGCCGCACCCCCTCTAGCGGGCGCGGGGCGAAGCGGTGCGGCG |
| | CCGGCAGGAAGGAAATGGGCGGGGAGGGCCTTCGTGCGTCGCCGCGCCGCCGTCCCCTTCTCCCTCTCCAGCCTCGG |
| | GGCTGTCCGCGGGGGGACGGCTGCCTTCGGGGGGGACGGGGCAGGGCGGGGTTCGGCTTCTGGCGTGTGACCGGCGG |
| | CTCTAGAGCCTCTGCTAACCTTGTTCTTGCCTTCTTCTTTTTCCTACAGCTCCTGGGCAACGTGCTGGTTATTGTGC |
| | TGTCTCATCATTTTGGCAAAGAATTA |
| SEQ ID NO: 18 | TCATAATCAACCTCTGGATTACAAAATTTGTGAAAGATTGACTGGTATTCTTAACTATGTTGCTCCTTTTACGCTAT |
| WPRE | GTGGATACGCTGCTTTAATGCCTTTGTATCATGCTATTGCTTCCCGTATGGCTTTCATTTTCTCCTCCTTGTATAAA |
| | TCCTGGTTGCTGTCTCTTTATGAGGAGTTGTGGCCCGTTGTCAGGCAACGTGGCGTGGTGTGCACTGTGTTTGCTGA |
| | CGCAACCCCCACTGGTTGGGGCATTGCCACCACCTGTCAGCTCCTTTCCGGGACTTTCGCTTTCCCCCTCCCTATTG |
| | CCACGGCGGAACTCATCGCCGCCTGCCTTGCCCGCTGCTGGACAGGGGCTCGGCTGTTGGGCACTGACAATTCCGTG |
| | GTGTTGTCGGGGAAATCATCGTCCTTTCCTTGGCTGCTCGCCTGTGTTGCCACCTGGATTCTGCGCGGGACGTCCTT |
| | CTGCTACGTCCCTTCGGCCCTCAATCCAGCGGACCTTCCTTCCCGCGGCCTGCTGCCGGCTCTGCGGCCTCTTCCGC |
| | GTCTTCGCCTTCGCCCTCAGACGAGTCGGATCTCCCTTTGGGCCGCCTCCCCGCTG |
| SEQ ID NO: 19 | CGACTGTGCCTTCTAGTTGCCAGCCATCTGTTGTTTGCCCCTCCCCCGTGCCTTCCTTGACCCTGGAAGGTGCCACT |
| bGHpA | CCCACTGTCCTTTCCTAATAAAATGAGGAAATTGCATCGCATTGTCTGAGTAGGTGTCATTCTATTCTGGGGGGTGG |
| | GGTGGGGCAGGACAGCAAGGGGGAGGATTGGGAAGACAATAGCAGGCATGCTGGGGA |
| SEQ ID NO: 20 | ATRRYYLGAVELSWDYMQSDLGELPVDARFPPRVPKSFPFNTSVVYKKTLFVEFTDHLFNIAKPRPPWMGLLGPTIQ |
| Amino acid | AEVYDTVVITLKNMASHPVSLHAVGVSYWKASEGAEYDDQTSQREKEDDKVFPGGSHTYVWQVLKENGPMASDPLCL |
| sequence of | TYSYLSHVDLVKDLNSGLIGALLVCREGSLAKEKTQTLHKFILLFAVFDEGKSWHSETKNSLMQDRDAASARAWPKM |
| wild type | HTVNGYVNRSLPGLIGCHRKSVYWHVIGMGTTPEVHSIFLEGHTFLVRNHRQASLEISPITFLTAQTLLMDLGQFLL |
| human | FCHISSHQHDGMEAYVKVDSCPEEPQLRMKNNEEAEDYDDDLTDSEMDVVRFDDDNSPSFIQIRSVAKKHPKTWVHY |
| mature FVIII | IAAEEEDWDYAPLVLAPDDRSYKSQYLNNGPQRIGRKYKKVRFMAYTDETFKTREAIQHESGILGPLLYGEVGDTLL |
| protein | IIFKNQASRPYNIYPHGITDVRPLYSRRLPKGVKHLKDFPILPGEIFKYKWTVTVEDGPTKSDPRCLTRYYSSFVNM |
| | ERDLASGLIGPLLICYKESVDQRGNQIMSDKRNVILFSVFDENRSWYLTENIQRFLPNPAGVQLEDPEFQASNIMHS |
| | INGYVFDSLQLSVCLHEVAYWYILSIGAQTDFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRSFSQNSRHPSTRQKQFNATTIPENDIEKTD |
| | PWFAHRTPMPKIQNVSSSDLLMLLRQSPTPHGLSLSDLQEAKYETFSDDPSPGAIDSNNSLSEMTHFRPQLHHSGDM |
| | VFTPESGLQLRLNEKLGTTAATELKKLDFKVSSTSNNLISTIPSDNLAAGTDNTSSLGPPSMPVHYDSQLDTTLFGK |
| | KSSPLTESGGPLSLSEENNDSKLLESGLMNSQESSWGKNVSSTESGRLFKGKRAHGPALLTKDNALFKVSISLLKTN |
| | KTSNNSATNRKTHIDGPSLLIENSPSVWQNILESDTEFKKVTPLIHDRMLMDKNATALRLNHMSNKTTSSKNMEMVQ |
| | QKKEGPIPPDAQNPDMSFFKMLFLPESARWIQRTHGKNSLNSGQGPSPKQLVSLGPEKSVEGQNFLSEKNKVVVGKG |
| | EFTKDVGLKEMVFPSSRNLFLTNLDNLHENNTHNQEKKIQEEIEKKETLIQENVVLPQIHTVTGTKNFMKNLFLLST |
| | RQNVEGSYDGAYAPVLQDFRSLNDSTNRTKKHTAHFSKKGEEENLEGLGNQTKQIVEKYACTTRISPNTSQQNFVTQ |
| | RSKRALKQFRLPLEETELEKRIIVDDTSTQWSKNMKHLTPSTLTQIDYNEKEKGAITQSPLSDCLTRSHSIPQANRS |
| | PLPIAKVSSFPSIRPIYLTRVLFQDNSSHLPAASYRKKDSGVQESSHFLQGAKKNNLSLAILTLEMTGDQREVGSLG |
| | TSATNSVTYKKVENTVLPKPDLPKTSGKVELLPKVHIYQKDLFPTETSNGSPGHLDLVEGSLLQGTEGAIKWNEANR |
| | PGKVPFLRVATESSAKTPSKLLDPLAWDNHYGTQIPKEEWKSQEKSPEKTAFKKKDTILSLNACESNHAIAAINEGQ |
| | NKPEIEVTWAKQGRTERLCSQNPPVLKRHQREITRTTLQSDQEEIDYDDTISVEMKKEDFDIYDEDENQSPRSFQKK |
| | TRHYFIAAVERLWDYGMSSSPHVLRNRAQSGSVPQFKKVVFQEFTDGSFTQPLYRGELNEHLGLLGPYIRAEVEDNI |
| | MVTFRNQASRPYSFYSSLISYEEDQRQGAEPRKNFVKPNETKTYFWKVQHHMAPTKDEFDCKAWAYFSDVDLEKDVH |
| | SGLIGPLLVCHTNTLNPAHGRQVTVQEFALFFTIFDETKSWYFTENMERNCRAPCNIQMEDPTFKENYRFHAINGYI |
| | MDTLPGLVMAQDQRIRWYLLSMGSNENIHSIHFSGHVFTVRKKEEYKMALYNLYPGVFETVEMLPSKAGIWRVECLI |
| | GEHLHAGMSTLFLVYSNKCQTPLGMASGHIRDFQITASGQYGQWAPKLARLHYSGSINAWSTKEPFSWIKVDLLAPM |
| | IIHGIKTQGARQKFSSLYISQFIIMYSLDGKKWQTYRGNSTGTLMVFFGNVDSSGIKHNIFNPPIIARYIRLHPTHY |
| | SIRSTLRMELMGCDLNSCSMPLGMESKAISDAQITASSYFTNMFATWSPSKARLHLQGRSNAWRPQVNNPKEWLQVD |
| | FQKTMKVTGVTTQGVKSLLTSMYVKEFLISSSQDGHQWTLFFQNGKVKVFQGNQDSFTPVVNSLDPPLLTRYLRIHP |
| | QSWVHQIALRMEVLGCEAQDLY |
| SEQ ID NO: 21 | CCAAATCAGATGCCGCCGGTCGCCGCCGGTAGGCGGGACTTCCGGTACAAGATGGCGGACAATTACGTCATTTCCTG |
| B19 WT 5′ | TGACGTCATTTCCTGTGACGTCACTTCCGGTGGGCGGGACTTCCGGAATTAGGGTTGGCTCTGGGCCAGCTTGCTTG |
| | GGGTTGCCTTGACACTAAGACAAGCGGCGCGCCGCTTGATCTTAGTGGCACGTCAACCCCAAGCGCTGGCCCAGAGC |
| | CAACCCTAATTCCGGAAGTCCCGCCCACCGGAAGTGACGTCACAGGAAATGACGTCACAGGAAATGACGTAATTGTC |
| | CGCCATCTTGTACCGGAAGTCCCGCCTACCGGCGGCGACCGGCGGCATCTGATTTGGTGTCTTCTTTTAAATTTT |
| SEQ ID NO: 22 | AAAATTTAAAAGAAGACACCAAATCAGATGCCGCCGGTCGCCGCCGGTAGGCGGGACTTCCGGTACAAGATGGCGGA |
| B19 WT 3′ | CAATTACGTCATTTCCTGTGACGTCATTTCCTGTGACGTCACTTCCGGTGGGCGGGACTTCCGGAATTAGGGTTGGC |
| | TCTGGGCCAGCGCTTGGGGTTGACGTGCCACTAAGATCAAGCGGCGCGCCGCTTGTCTTAGTGTCAAGGCAACCCCA |
| | AGCAAGCTGGCCCAGAGCCAACCCTAATTCCGGAAGTCCCGCCCACCGGAAGTGACGTCACAGGAAATGACGTCACA |
| | GGAAATGACGTAATTGTCCGCCATCTTGTACCGGAAGTCCCGCCTACCGGCGGCGACCGGCGGCATCTGATTTGG |
| SEQ ID NO: 23 | CCAAATCAGATGCCGCCGGTCGCCGCCGGTAGGCGGGACTTCCGGTACAGCGCGCCGCTGTACCGGAAGTCCCGCCT |
| 5′_B19_minimal | ACCGGCGGCGACCGGCGGCATCTGATTTGGTGTCTTCTTTTAAATTTT |
| SEQ ID NO: 24 | AAAATTTAAAAGAAGACACCAAATCAGATGCCGCCGGTCGCCGCCGGTAGGCGGGACTTCCGGTACAGCGGCGCGCT |
| 3′_B19_minimal | GTACCGGAAGTCCCGCCTACCGGCGGCGACCGGCGGCATCTGATTTGG |
| SEQ ID NO: 25 | CTCATTGGAGGGTTCGTTCGTTCGAACGTTCGTTCGCATGCGAACGAACGTTCGAACGAACGAACCCTCCAATGAGA |
| 5′_GPV_minimal | CTCAAGGACAAGAGGATATTTTGCGCGCCAGGAAGTG |
| SEQ ID NO: 26 | CACTTCCTGGCGCGCAAAATATCCTCTTGTCCTTGAGTCTCATTGGAGGGTTCGTTCGTTCGAACGTTCGTTCGCAT |
| 3′_GPV_minimal | GCGAACGAACGTTCGAACGAACGAACCCTCCAATGAG |
| SEQ ID NO: 27 | CTCATTGGAGGGTTCGTTCGTTCGAACCAGCCAATCAGGGGAGGGGGAAGTGACGCAAGTTCCGGTCACATGCTTCC |
| 5′_GPV_A186 | GGTGACGCACATCCGGTGACGTAGTTCGCATGCCTGTCTATCGCCTACCCATCCCTGTCTGAGATCAAGGGCGTGAT |
| | CGTGCACAGACTGGAGAGCGTGTCCTATAATATCGGCTCTCAGGAGTGGAGCACCACAGTGCCCAGATACGTGGCCA |
| | CCCAGGGCTATCTGATCTCCAACTTCGACGCATGCGAACTACGTCACCGGATGTGCGTCACCGGAAGCATGTGACCG |
| | GAACTTGCGTCACTTCCCCCTCCCCTGATTGGCTGGTTCGAACGAACGAACCCTCCAATGAGACTCAAGGACAAGAG |
| | GATATTTTGCGCGCCAGGAAGTG |
| SEQ ID NO: 28 | CACTTCCTGGCGCGCAAAATATCCTCTTGTCCTTGAGTCTCATTGGAGGGTTCGTTCGTTCGAACCAGCCAATCAGG |
| 3′_GPV_A186 | GGAGGGGGAAGTGACGCAAGTTCCGGTCACATGCTTCCGGTGACGCACATCCGGTGACGTAGTTCGCATGCCTGTCT |
| | ATCGCCTACCCATCCCTGTCTGAGATCAAGGGCGTGATCGTGCACAGACTGGAGAGCGTGTCCTATAATATCGGCTC |
| | TCAGGAGTGGAGCACCACAGTGCCCAGATACGTGGCCACCCAGGGCTATCTGATCTCCAACTTCGACGCATGCGAAC |
| | TACGTCACCGGATGTGCGTCACCGGAAGCATGTGACCGGAACTTGCGTCACTTCCCCCTCCCCTGATTGGCTGGTTC |
| | GAACGAACGAACCCTCCAATGAG |
| SEQ ID NO: 29 | CTCATTGGAGGGTTCGTTCGTTCGAACCAGCCAATCAGGGGAGGGGGAAGTGACGCAAGTTCCGGTCACATGCTTCC |
| 5′_GPV_A120 | GGTGACGCACATCCGGTGACGTAGTTCCGGTCACGTGCTTCCTGTCACGTGTTTCCGGTCGCATGCCTGTCTATCGC |
| | CTACCCATCCCTGTCTGAGATCAAGGGCGTGATCGTGCACAGACTGGAGAGCGTGTCCTATAATATCGGCTCTCAGG |
| | AGTGGAGCACCACAGTGCCCAGATACGTGGCCACCCAGGGCTATCTGATCTCCAACTTCGACGCATGCTCACGTGAC |
| | CGGAAACACGTGACAGGAAGCACGTGACCGGAACTACGTCACCGGATGTGCGTCACCGGAAGCATGTGACCGGAACT |
| | TGCGTCACTTCCCCCTCCCCTGATTGGCTGGTTCGAACGAACGAACCCTCCAATGAGACTCAAGGACAAGAGGATAT |
| | TTTGCGCGCCAGGAAGTG |
| SEQ ID NO: 30 | CACTTCCTGGCGCGCAAAATATCCTCTTGTCCTTGAGTCTCATTGGAGGGTTCGTTCGTTCGAACCAGCCAATCAGG |
| 3′_GPV_A120 | GGAGGGGGAAGTGACGCAAGTTCCGGTCACATGCTTCCGGTGACGCACATCCGGTGACGTAGTTCCGGTCACGTGCT |
| | TCCTGTCACGTGTTTCCGGTCACGTGAGCATGCCTGTCTATCGCCTACCCATCCCTGTCTGAGATCAAGGGCGTGAT |
| | CGTGCACAGACTGGAGAGCGTGTCCTATAATATCGGCTCTCAGGAGTGGAGCACCACAGTGCCCAGATACGTGGCCA |
| | CCCAGGGCTATCTGATCTCCAACTTCGACGGCATGCGACCGGAAACACGTGACAGGAAGCACGTGACCGGAACTACG |
| | TCACCGGATGTGCGTCACCGGAAGCATGTGACCGGAACTTGCGTCACTTCCCCCTCCCCTGATTGGCTGGTTCGAAC |
| | GAACGAACCCTCCAATGAG |
| SEQ ID NO: 31 | ATGCAAATAGAGCTCTCCACCTGCTTCTTTCTGTGCCTTTTGCGATTCTGCTTTAGTGCCACCAGAAGATACTACCT |
| Nucleic acid | GGGTGCAGTGGAACTGTCATGGGACTATATGCAAAGTGATCTCGGTGAGCTGCCTGTGGACGCAAGATTTCCTCCTA |
| sequence of | GAGTGCCAAAATCTTTTCCATTCAACACCTCAGTCGTGTACAAAAAGACTCTGTTTGTAGAATTCACGGATCACCTT |
| wild type | TTCAACATCGCTAAGCCAAGGCCACCCTGGATGGGTCTGCTAGGTCCTACCATCCAGGCTGAGGTTTATGATACAGT |
| human FVIII | GGTCATTACACTTAAGAACATGGCTTCCCATCCTGTCAGTCTTCATGCTGTTGGTGTATCCTACTGGAAAGCTTCTG |
| | AGGGAGCTGAATATGATGATCAGACCAGTCAAAGGGAGAAAGAAGATGATAAAGTCTTCCCTGGTGGAAGCCATACA |
| | TATGTCTGGCAGGTCCTGAAAGAGAATGGTCCAATGGCCTCTGACCCACTGTGCCTTACCTACTCATATCTTTCTCA |
| | TGTGGACCTGGTAAAAGACTTGAATTCAGGCCTCATTGGAGCCCTACTAGTATGTAGAGAAGGGAGTCTGGCCAAGG |
| | AAAAGACACAGACCTTGCACAAATTTATACTACTTTTTGCTGTATTTGATGAAGGGAAAAGTTGGCACTCAGAAACA |
| | AAGAACTCCTTGATGCAGGATAGGGATGCTGCATCTGCTCGGGCCTGGCCTAAAATGCACACAGTCAATGGTTATGT |
| | AAACAGGTCTCTGCCAGGTCTGATTGGATGCCACAGGAAATCAGTCTATTGGCATGTGATTGGAATGGGCACCACTC |
| | CTGAAGTGCACTCAATATTCCTCGAAGGTCACACATTTCTTGTGAGGAACCATCGCCAGGCGTCCTTGGAAATCTCG |
| | CCAATAACTTTCCTTACTGCTCAAACACTCTTGATGGACCTTGGACAGTTTCTACTGTTTTGTCATATCTCTTCCCA |
| | CCAACATGATGGCATGGAAGCTTATGTCAAAGTAGACAGCTGTCCAGAGGAACCCCAACTACGAATGAAAAATAATG |
| | AAGAAGCGGAAGACTATGATGATGATCTTACTGATTCTGAAATGGATGTGGTCAGGTTTGATGATGACAACTCTCCT |
| | TCCTTTATCCAAATTCGCTCAGTTGCCAAGAAGCATCCTAAAACTTGGGTACATTACATTGCTGCTGAAGAGGAGGA |
| | CTGGGACTATGCTCCCTTAGTCCTCGCCCCCGATGACAGAAGTTATAAAAGTCAATATTTGAACAATGGCCCTCAGC |
| | GGATTGGTAGGAAGTACAAAAAAGTCCGATTTATGGCATACACAGATGAAACCTTTAAGACTCGTGAAGCTATTCAG |
| | CATGAATCAGGAATCTTGGGACCTTTACTTTATGGGGAAGTTGGAGACACACTGTTGATTATATTTAAGAATCAAGC |
| | AAGCAGACCATATAACATCTACCCTCACGGAATCACTGATGTCCGTCCTTTGTATTCAAGGAGATTACCAAAAGGTG |
| | TAAAACATTTGAAGGATTTTCCAATTCTGCCAGGAGAAATATTCAAATATAAATGGACAGTGACTGTAGAAGATGGG |
| | CCAACTAAATCAGATCCTCGGTGCCTGACCCGCTATTACTCTAGTTTCGTTAATATGGAGAGAGATCTAGCTTCAGG |
| | ACTCATTGGCCCTCTCCTCATCTGCTACAAAGAATCTGTAGATCAAAGAGGAAACCAGATAATGTCAGACAAGAGGA |
| | ATGTCATCCTGTTTTCTGTATTTGATGAGAACCGAAGCTGGTACCTCACAGAGAATATACAACGCTTTCTCCCCAAT |
| | CCAGCTGGAGTGCAGCTTGAGGATCCAGAGTTCCAAGCCTCCAACATCATGCACAGCATCAATGGCTATGTTTTTGA |
| | TAGTTTGCAGTTGTCAGTTTGTTTGCATGAGGTGGCATACTGGTACATTCTAAGCATTGGAGCACAGACTGACTTCC |
| | TTTCTGTCTTCTTCTCTGGATATACCTTCAAACACAAAATGGTCTATGAAGACACACTCACCCTATTCCCATTCTCA |
| | GGAGAAACTGTCTTCATGTCGATGGAAAACCCAGGTCTATGGATTCTGGGGTGCCACAACTCAGACTTTCGGAACAG |
| | AGGCATGACCGCCTTACTGAAGGTTTCTAGTTGTGACAAGAACACTGGTGATTATTACGAGGACAGTTATGAAGATA |
| | TTTCAGCATACTTGCTGAGTAAAAACAATGCCATTGAACCAAGAAGCTTCTCCCAGAATTCAAGACACCCTAGCACT |
| | AGGCAAAAGCAATTTAATGCCACCACAATTCCAGAAAATGACATAGAGAAGACTGACCCTTGGTTTGCACACAGAAC |
| | ACCTATGCCTAAAATACAAAATGTCTCCTCTAGTGATTTGTTGATGCTCTTGCGACAGAGTCCTACTCCACATGGGC |
| | TATCCTTATCTGATCTCCAAGAAGCCAAATATGAGACTTTTTCTGATGATCCATCACCTGGAGCAATAGACAGTAAT |
| | AACAGCCTGTCTGAAATGACACACTTCAGGCCACAGCTCCATCACAGTGGGGACATGGTATTTACCCCTGAGTCAGG |
| | CCTCCAATTAAGATTAAATGAGAAACTGGGGACAACTGCAGCAACAGAGTTGAAGAAACTTGATTTCAAAGTTTCTA |
| | GTACATCAAATAATCTGATTTCAACAATTCCATCAGACAATTTGGCAGCAGGTACTGATAATACAAGTTCCTTAGGA |
| | CCCCCAAGTATGCCAGTTCATTATGATAGTCAATTAGATACCACTCTATTTGGCAAAAAGTCATCTCCCCTTACTGA |
| | GTCTGGTGGACCTCTGAGCTTGAGTGAAGAAAATAATGATTCAAAGTTGTTAGAATCAGGTTTAATGAATAGCCAAG |
| | AAAGTTCATGGGGAAAAAATGTATCGTCAACAGAGAGTGGTAGGTTATTTAAAGGGAAAAGAGCTCATGGACCTGCT |
| | TTGTTGACTAAAGATAATGCCTTATTCAAAGTTAGCATCTCTTTGTTAAAGACAAACAAAACTTCCAATAATTCAGC |
| | AACTAATAGAAAGACTCACATTGATGGCCCATCATTATTAATTGAGAATAGTCCATCAGTCTGGCAAAATATATTAG |
| | AAAGTGACACTGAGTTTAAAAAAGTGACACCTTTGATTCATGACAGAATGCTTATGGACAAAAATGCTACAGCTTTG |
| | AGGCTAAATCATATGTCAAATAAAACTACTTCATCAAAAAACATGGAAATGGTCCAACAGAAAAAAGAGGGCCCCAT |
| | TCCACCAGATGCACAAAATCCAGATATGTCGTTCTTTAAGATGCTATTCTTGCCAGAATCAGCAAGGTGGATACAAA |
| | GGACTCATGGAAAGAACTCTCTGAACTCTGGGCAAGGCCCCAGTCCAAAGCAATTAGTATCCTTAGGACCAGAAAAA |
| | TCTGTGGAAGGTCAGAATTTCTTGTCTGAGAAAAACAAAGTGGTAGTAGGAAAGGGTGAATTTACAAAGGACGTAGG |
| | ACTCAAAGAGATGGTTTTTCCAAGCAGCAGAAACCTATTTCTTACTAACTTGGATAATTTACATGAAAATAATACAC |
| | ACAATCAAGAAAAAAAAATTCAGGAAGAAATAGAAAAGAAGGAAACATTAATCCAAGAGAATGTAGTTTTGCCTCAG |
| | ATACATACAGTGACTGGCACTAAGAATTTCATGAAGAACCTTTTCTTACTGAGCACTAGGCAAAATGTAGAAGGTTC |
| | ATATGACGGGGCATATGCTCCAGTACTTCAAGATTTTAGGTCATTAAATGATTCAACAAATAGAACAAAGAAACACA |
| | CAGCTCATTTCTCAAAAAAAGGGGAGGAAGAAAACTTGGAAGGCTTGGGAAATCAAACCAAGCAAATTGTAGAGAAA |
| | TATGCATGCACCACAAGGATATCTCCTAATACAAGCCAGCAGAATTTTGTCACGCAACGTAGTAAGAGAGCTTTGAA |
| | ACAATTCAGACTCCCACTAGAAGAAACAGAACTTGAAAAAAGGATAATTGTGGATGACACCTCAACCCAGTGGTCCA |
| | AAAACATGAAACATTTGACCCCGAGCACCCTCACACAGATAGACTACAATGAGAAGGAGAAAGGGGCCATTACTCAG |
| | TCTCCCTTATCAGATTGCCTTACGAGGAGTCATAGCATCCCTCAAGCAAATAGATCTCCATTACCCATTGCAAAGGT |
| | ATCATCATTTCCATCTATTAGACCTATATATCTGACCAGGGTCCTATTCCAAGACAACTCTTCTCATCTTCCAGCAG |
| | CATCTTATAGAAAGAAAGATTCTGGGGTCCAAGAAAGCAGTCATTTCTTACAAGGAGCCAAAAAAAATAACCTTTCT |
| | TTAGCCATTCTAACCTTGGAGATGACTGGTGATCAAAGAGAGGTTGGCTCCCTGGGGACAAGTGCCACAAATTCAGT |
| | CACATACAAGAAAGTTGAGAACACTGTTCTCCCGAAACCAGACTTGCCCAAAACATCTGGCAAAGTTGAATTGCTTC |
| | CAAAAGTTCACATTTATCAGAAGGACCTATTCCCTACGGAAACTAGCAATGGGTCTCCTGGCCATCTGGATCTCGTG |
| | GAAGGGAGCCTTCTTCAGGGAACAGAGGGAGCGATTAAGTGGAATGAAGCAAACAGACCTGGAAAAGTTCCCTTTCT |
| | GAGAGTAGCAACAGAAAGCTCTGCAAAGACTCCCTCCAAGCTATTGGATCCTCTTGCTTGGGATAACCACTATGGTA |
| | CTCAGATACCAAAAGAAGAGTGGAAATCCCAAGAGAAGTCACCAGAAAAAACAGCTTTTAAGAAAAAGGATACCATT |
| | TTGTCCCTGAACGCTTGTGAAAGCAATCATGCAATAGCAGCAATAAATGAGGGACAAAATAAGCCCGAAATAGAAGT |
| | CACCTGGGCAAAGCAAGGTAGGACTGAAAGGCTGTGCTCTCAAAACCCACCAGTCTTGAAACGCCATCAACGGGAAA |
| | TAACTCGTACTACTCTTCAGTCAGATCAAGAGGAAATTGACTATGATGATACCATATCAGTTGAAATGAAGAAGGAA |
| | GATTTTGACATTTATGATGAGGATGAAAATCAGAGCCCCCGCAGCTTTCAAAAGAAAACACGACACTATTTTATTGC |
| | TGCAGTGGAGAGGCTCTGGGATTATGGGATGAGTAGCTCCCCACATGTTCTAAGAAACAGGGCTCAGAGTGGCAGTG |
| | TCCCTCAGTTCAAGAAAGTTGTTTTCCAGGAATTTACTGATGGCTCCTTTACTCAGCCCTTATACCGTGGAGAACTA |
| | AATGAACATTTGGGACTCCTGGGGCCATATATAAGAGCAGAAGTTGAAGATAATATCATGGTAACTTTCAGAAATCA |
| | GGCCTCTCGTCCCTATTCCTTCTATTCTAGCCTTATTTCTTATGAGGAAGATCAGAGGCAAGGAGCAGAACCTAGAA |
| | AAAACTTTGTCAAGCCTAATGAAACCAAAACTTACTTTTGGAAAGTGCAACATCATATGGCACCCACTAAAGATGAG |
| | TTTGACTGCAAAGCCTGGGCTTATTTCTCTGATGTTGACCTGGAAAAAGATGTGCACTCAGGCCTGATTGGACCCCT |
| | TCTGGTCTGCCACACTAACACACTGAACCCTGCTCATGGGAGACAAGTGACAGTACAGGAATTTGCTCTGTTTTTCA |
| | CCATCTTTGATGAGACCAAAAGCTGGTACTTCACTGAAAATATGGAAAGAAACTGCAGGGCTCCCTGCAATATCCAG |
| | ATGGAAGATCCCACTTTTAAAGAGAATTATCGCTTCCATGCAATCAATGGCTACATAATGGATACACTACCTGGCTT |
| | AGTAATGGCTCAGGATCAAAGGATTCGATGGTATCTGCTCAGCATGGGCAGCAATGAAAACATCCATTCTATTCATT |
| | TCAGTGGACATGTGTTCACTGTACGAAAAAAAGAGGAGTATAAAATGGCACTGTACAATCTCTATCCAGGTGTTTTT |
| | GAGACAGTGGAAATGTTACCATCCAAAGCTGGAATTTGGCGGGTGGAATGCCTTATTGGCGAGCATCTACATGCTGG |
| | GATGAGCACACTTTTTCTGGTGTACAGCAATAAGTGTCAGACTCCCCTGGGAATGGCTTCTGGACACATTAGAGATT |
| | TTCAGATTACAGCTTCAGGACAATATGGACAGTGGGCCCCAAAGCTGGCCAGACTTCATTATTCCGGATCAATCAAT |
| | GCCTGGAGCACCAAGGAGCCCTTTTCTTGGATCAAGGTGGATCTGTTGGCACCAATGATTATTCACGGCATCAAGAC |
| | CCAGGGTGCCCGTCAGAAGTTCTCCAGCCTCTACATCTCTCAGTTTATCATCATGTATAGTCTTGATGGGAAGAAGT |
| | GGCAGACTTATCGAGGAAATTCCACTGGAACCTTAATGGTCTTCTTTGGCAATGTGGATTCATCTGGGATAAAACAC |
| | AATATTTTTAACCCTCCAATTATTGCTCGATACATCCGTTTGCACCCAACTCATTATAGCATTCGCAGCACTCTTCG |
| | CATGGAGTTGATGGGCTGTGATTTAAATAGTTGCAGCATGCCATTGGGAATGGAGAGTAAAGCAATATCAGATGCAC |
| | AGATTACTGCTTCATCCTACTTTACCAATATGTTTGCCACCTGGTCTCCTTCAAAAGCTCGACTTCACCTCCAAGGG |
| | AGGAGTAATGCCTGGAGACCTCAGGTGAATAATCCAAAAGAGTGGCTGCAAGTGGACTTCCAGAAGACAATGAAAGT |
| | CACAGGAGTAACTACTCAGGGAGTAAAATCTCTGCTTACCAGCATGTATGTGAAGGAGTTCCTCATCTCCAGCAGTC |
| | AAGATGGCCATCAGTGGACTCTCTTTTTTCAGAATGGCAAAGTAAAGGTTTTTCAGGGAAATCAAGACTCCTTCACA |
| | CCTGTGGTGAACTCTCTAGACCCACCGTTACTGACTCGCTACCTTCGAATTCACCCCCAGAGTTGGGTGCACCAGAT |
| | TGCCCTGAGGATGGAGGTTCTGGGCTGCGAGGCACAGGACCTCTAC |
| SEQ ID NO: 32 | GCCACTCGCCGGTACTACCTTGGAGCCGTGGAGCTTTCATGGGACTACATGCAGAGCGACCTGGGCGAACTCCCCGT |
| Nucleotide | GGATGCCAGATTCCCCCCCCGCGTGCCAAAGTCCTTCCCCTTTAACACCTCCGTGGTGTACAAGAAAACCCTCTTTG |
| sequence | TCGAGTTCACTGACCACCTGTTCAACATCGCCAAGCCGCGCCCACCTTGGATGGGCCTCCTGGGACCGACCATTCAA |
| encoding | GCTGAAGTGTACGACACCGTGGTGATCACCCTGAAGAACATGGCGTCCCACCCCGTGTCCCTGCATGCGGTCGGAGT |
| BDD-CO6FVIII | GTCCTACTGGAAGGCCTCCGAAGGAGCTGAGTACGACGACCAGACTAGCCAGCGGGAAAAGGAGGACGATAAAGTGT |
| (V1.0) | TCCCGGGCGGCTCGCATACTTACGTGTGGCAAGTCCTGAAGGAAAACGGACCTATGGCATCCGATCCTCTGTGCCTG |
| (no XTEN) | ACTTACTCCTACCTTTCCCATGTGGACCTCGTGAAGGACCTGAACAGCGGGCTGATTGGTGCACTTCTCGTGTGCCG |
| | CGAAGGTTCGCTCGCTAAGGAAAAGACCCAGACCCTCCATAAGTTCATCCTTTTGTTCGCTGTGTTCGATGAAGGAA |
| | AGTCATGGCATTCCGAAACTAAGAACTCGCTGATGCAGGACCGGGATGCCGCCTCAGCCCGCGCCTGGCCTAAAATG |
| | CATACAGTCAACGGATACGTGAATCGGTCACTGCCCGGGCTCATCGGTTGTCACAGAAAGTCCGTGTACTGGCACGT |
| | CATCGGCATGGGCACTACGCCTGAAGTGCACTCCATCTTCCTGGAAGGGCACACCTTCCTCGTGCGCAACCACCGCC |
| | AGGCCTCTCTGGAAATCTCCCCGATTACCTTTCTGACCGCCCAGACTCTGCTCATGGACCTGGGGCAGTTCCTTCTC |
| | TTCTGCCACATCTCCAGCCATCAGCACGACGGAATGGAGGCCTACGTGAAGGTGGACTCATGCCCGGAAGAACCTCA |
| | GTTGCGGATGAAGAACAACGAGGAGGCCGAGGACTATGACGACGATTTGACTGACTCCGAGATGGACGTCGTGCGGT |
| | TCGATGACGACAACAGCCCCAGCTTCATCCAGATTCGCAGCGTGGCCAAGAAGCACCCCAAAACCTGGGTGCACTAC |
| | ATCGCGGCCGAGGAAGAAGATTGGGACTACGCCCCGTTGGTGCTGGCACCCGATGACCGGTCGTACAAGTCCCAGTA |
| | TCTGAACAATGGTCCGCAGCGGATTGGCAGAAAGTACAAGAAAGTGCGGTTCATGGCGTACACTGACGAAACGTTTA |
| | AGACCCGGGAGGCCATTCAACATGAGAGCGGCATTCTGGGACCACTGCTGTACGGAGAGGTCGGCGATACCCTGCTC |
| | ATCATCTTCAAAAACCAGGCCTCCCGGCCTTACAACATCTACCCTCACGGAATCACCGACGTGCGGCCACTCTACTC |
| | GCGGCGCCTGCCGAAGGGCGTCAAGCACCTGAAAGACTTCCCTATCCTGCCGGGCGAAATCTTCAAGTATAAGTGGA |
| | CCGTCACCGTGGAGGACGGGCCCACCAAGAGCGATCCTAGGTGTCTGACTCGGTACTACTCCAGCTTCGTGAACATG |
| | GAACGGGACCTGGCATCGGGACTCATTGGACCGCTGCTGATCTGCTACAAAGAGTCGGTGGATCAACGCGGCAACCA |
| | GATCATGTCCGACAAGCGCAACGTGATCCTGTTCTCCGTGTTTGATGAAAACAGATCCTGGTACCTCACTGAAAACA |
| | TCCAGAGGTTCCTCCCAAACCCCGCAGGAGTGCAACTGGAGGACCCTGAGTTTCAGGCCTCGAATATCATGCACTCG |
| | ATTAACGGTTACGTGTTCGACTCGCTGCAGCTGAGCGTGTGCCTCCATGAAGTCGCTTACTGGTACATTCTGTCCAT |
| | CGGCGCCCAGACTGACTTCCTGAGCGTGTTCTTTTCCGGTTACACCTTTAAGCACAAGATGGTGTACGAAGATACCC |
| | TGACCCTGTTCCCTTTCTCCGGCGAAACGGTGTTCATGTCGATGGAGAACCCGGGTCTGTGGATTCTGGGATGCCAC |
| | AACAGCGACTTTCGGAACCGCGGAATGACTGCCCTGCTGAAGGTGTCCTCATGCGACAAGAACACCGGAGACTACTA |
| | CGAGGACTCCTACGAGGATATCTCAGCCTACCTCCTGTCCAAGAACAACGCGATCGAGCCGCGCAGCTTCAGCCAGA |
| | ACCCGCCTGTGCTGAAGAGGCACCAGCGAGAAATTACCCGGACCACCCTCCAATCGGATCAGGAGGAAATCGACTAC |
| | GACGACACCATCTCGGTGGAAATGAAGAAGGAAGATTTCGATATCTACGACGAGGACGAAAATCAGTCCCCTCGCTC |
| | ATTCCAAAAGAAAACTAGACACTACTTTATCGCCGCGGTGGAAAGACTGTGGGACTATGGAATGTCATCCAGCCCTC |
| | ACGTCCTTCGGAACCGGGCCCAGAGCGGATCGGTGCCTCAGTTCAAGAAAGTGGTGTTCCAGGAGTTCACCGACGGC |
| | AGCTTCACCCAGCCGCTGTACCGGGGAGAACTGAACGAACACCTGGGCCTGCTCGGTCCCTACATCCGCGCGGAAGT |
| | GGAGGATAACATCATGGTGACCTTCCGTAACCAAGCATCCAGACCTTACTCCTTCTATTCCTCCCTGATCTCATACG |
| | AGGAGGACCAGCGCCAAGGCGCCGAGCCCCGCAAGAACTTCGTCAAGCCCAACGAGACTAAGACCTACTTCTGGAAG |
| | GTCCAACACCATATGGCCCCGACCAAGGATGAGTTTGACTGCAAGGCCTGGGCCTACTTCTCCGACGTGGACCTTGA |
| | GAAGGATGTCCATTCCGGCCTGATCGGGCCGCTGCTCGTGTGTCACACCAACACCCTGAACCCAGCGCATGGACGCC |
| | AGGTCACCGTCCAGGAGTTTGCTCTGTTCTTCACCATTTTTGACGAAACTAAGTCCTGGTACTTCACCGAGAATATG |
| | GAGCGAAACTGTAGAGCGCCCTGCAATATCCAGATGGAAGATCCGACTTTCAAGGAGAACTATAGATTCCACGCCAT |
| | CAACGGGTACATCATGGATACTCTGCCGGGGCTGGTCATGGCCCAGGATCAGAGGATTCGGTGGTACTTGCTGTCAA |
| | TGGGATCGAACGAAAACATTCACTCCATTCACTTCTCCGGTCACGTGTTCACTGTGCGCAAGAAGGAGGAGTACAAG |
| | ATGGCGCTGTACAATCTGTACCCCGGGGTGTTCGAAACTGTGGAGATGCTGCCGTCCAAGGCCGGCATCTGGAGAGT |
| | GGAGTGCCTGATCGGAGAGCACCTCCACGCGGGGATGTCCACCCTCTTCCTGGTGTACTCGAATAAGTGCCAGACCC |
| | CGCTGGGCATGGCCTCGGGCCACATCAGAGACTTCCAGATCACAGCAAGCGGACAATACGGCCAATGGGCGCCGAAG |
| | CTGGCCCGCTTGCACTACTCCGGATCGATCAACGCATGGTCCACCAAGGAACCGTTCTCGTGGATTAAGGTGGACCT |
| | CCTGGCCCCTATGATTATCCACGGAATTAAGACCCAGGGCGCCAGGCAGAAGTTCTCCTCCCTGTACATCTCGCAAT |
| | TCATCATCATGTACAGCCTGGACGGGAAGAAGTGGCAGACTTACAGGGGAAACTCCACCGGCACCCTGATGGTCTTT |
| | TTCGGCAACGTGGATTCCTCCGGCATTAAGCACAACATCTTCAACCCACCGATCATAGCCAGATATATTAGGCTCCA |
| | CCCCACTCACTACTCAATCCGCTCAACTCTTCGGATGGAACTCATGGGGTGCGACCTGAACTCCTGCTCCATGCCGT |
| | TGGGGATGGAATCAAAGGCTATTAGCGACGCCCAGATCACCGCGAGCTCCTACTTCACTAACATGTTCGCCACCTGG |
| | AGCCCCTCCAAGGCCAGGCTGCACTTGCAGGGACGGTCAAATGCCTGGCGGCCGCAAGTGAACAATCCGAAGGAATG |
| | GCTTCAAGTGGATTTCCAAAAGACCATGAAAGTGACCGGAGTCACCACCCAGGGAGTGAAGTCCCTTCTGACCTCGA |
| | TGTATGTGAAGGAGTTCCTGATTAGCAGCAGCCAGGACGGGCACCAGTGGACCCTGTTCTTCCAAAACGGAAAGGTC |
| | AAGGTGTTCCAGGGGAACCAGGACTCGTTCACACCCGTGGTGAACTCCCTGGACCCCCCACTGCTGACGCGGTACTT |
| | GAGGATTCATCCTCAGTCCTGGGTCCATCAGATTGCATTGCGAATGGAAGTCCTGGGCTGCGAGGCCCAGGACCTGT |
| | ACTGA |
| SEQ ID NO: 33 | GCCACCCGCCGGTATTACTTAGGTGCTGTGGAACTGAGCTGGGACTACATGCAGTCCGACCTGGGAGAACTGCCGGT |
| Nucleotide | GGACGCGAGATTCCCACCTAGAGTCCCGAAGTCCTTCCCATTCAACACCTCCGTGGTCTACAAAAAGACCCTGTTCG |
| sequence | TGGAGTTCACTGACCACCTTTTCAATATTGCCAAGCCGCGCCCCCCCTGGATGGGCCTGCTTGGTCCTACGATCCAA |
| encoding | GCAGAGGTCTACGACACCGTGGTCATCACACTGAAGAACATGGCCTCACACCCCGTGTCGCTGCATGCTGTGGGAGT |
| coBDDFVIII | GTCCTACTGGAAGGCCTCAGAGGGTGCCGAATATGATGACCAGACCAGCCAGAGGGAAAAGGAGGATGACAAAGTGT |
| (V2.0) | TCCCGGGTGGCAGCCACACTTACGTGTGGCAAGTGCTGAAGGAAAACGGGCCTATGGCGTCGGACCCCCTATGCCTG |
| (no XTEN) | ACCTACTCCTACCTGTCCCATGTGGACCTTGTGAAGGATCTCAACTCGGGACTGATCGGCGCCCTCTTGGTGTGCAG |
| | AGAAGGCAGCCTGGCGAAGGAAAAGACTCAGACCCTGCACAAGTTCATTCTGTTGTTTGCTGTGTTCGATGAAGGAA |
| | AGTCCTGGCACTCAGAAACCAAGAACTCGCTGATGCAGGATAGAGATGCGGCCTCGGCCAGAGCCTGGCCTAAAATG |
| | CACACCGTCAACGGATATGTGAACAGGTCGCTCCCTGGCCTCATCGGCTGCCACAGAAAGTCCGTGTATTGGCATGT |
| | GATCGGCATGGGTACTACTCCGGAAGTGCATAGTATCTTTCTGGAGGGCCATACCTTCTTGGTGCGCAACCACAGAC |
| | AGGCCTCGCTGGAAATCTCGCCTATCACTTTCTTGACTGCGCAGACCCTCCTTATGGACCTTGGACAGTTCCTGCTG |
| | TTCTGTCACATCAGCTCCCATCAGCATGATGGGATGGAGGCCTATGTCAAAGTGGACTCCTGCCCTGAGGAGCCACA |
| | GCTCCGGATGAAGAACAATGAGGAAGCGGAGGATTACGACGACGACCTGACTGACAGCGAAATGGACGTCGTGCGAT |
| | TCGATGACGACAACAGCCCGTCCTTCATCCAAATTAGATCAGTGGCGAAGAAGCACCCCAAGACCTGGGTGCACTAC |
| | ATTGCCGCCGAGGAAGAGGACTGGGACTACGCGCCGCTGGTGCTGGCGCCAGACGACAGGAGCTACAAGTCCCAGTA |
| | CCTCAACAACGGGCCGCAGCGCATTGGCAGGAAGTACAAGAAAGTCCGCTTCATGGCCTACACTGATGAAACCTTCA |
| | AGACGAGGGAAGCCATCCAGCACGAGTCAGGCATCCTGGGACCGCTCCTTTACGGCGAAGTCGGGGATACCCTGCTC |
| | ATCATTTTCAAGAACCAGGCATCGCGGCCCTACAACATCTACCCTCACGGGATCACAGACGTGCGCCCGCTCTACTC |
| | CCGCCGGCTGCCCAAGGGAGTGAAGCACCTGAAGGATTTTCCCATCCTGCCGGGAGAAATCTTCAAGTACAAGTGGA |
| | CCGTGACTGTGGAAGATGGCCCTACCAAGTCGGACCCTCGCTGTCTGACCCGGTACTATTCCTCGTTTGTGAACATG |
| | GAGCGCGACCTGGCCTCGGGGCTGATTGGTCCGCTGCTGATCTGCTACAAGGAGTCCGTGGACCAGCGCGGGAACCA |
| | GATCATGTCCGACAAGCGCAACGTGATCCTGTTCTCTGTCTTTGATGAAAACAGATCGTGGTACTTGACTGAGAATA |
| | TCCAGCGGTTCCTGCCCAACCCAGCGGGAGTGCAACTGGAGGACCCGGAGTTCCAGGCCTCAAACATTATGCACTCT |
| | ATCAACGGCTATGTGTTCGACTCGCTCCAACTGAGCGTGTGCCTGCATGAAGTGGCATACTGGTACATTCTGTCCAT |
| | CGGAGCCCAGACCGACTTCCTGTCCGTGTTCTTCTCCGGATACACCTTCAAGCATAAGATGGTGTACGAGGACACTC |
| | TGACCCTCTTCCCATTTTCCGGAGAAACTGTGTTCATGTCAATGGAAAACCCGGGCTTGTGGATTCTGGGTTGCCAT |
| | AACTCGGACTTCCGGAATAGAGGGATGACCGCCCTGCTGAAAGTGTCCAGCTGTGACAAGAATACCGGCGATTACTA |
| | CGAGGACAGCTATGAGGACATCTCCGCTTATCTGCTGTCCAAGAACAACGCCATTGAACCCAGGTCCTTCTCCCAAA |
| | ACGGTGCACCGGCCTCATCCCCCCCCGTGCTGAAGCGGCATCAAAGAGAGATCACCAGGACCACTCTCCAGTCCGAT |
| | CAGGAAGAAATTGACTACGACGATACTATCAGCGTGGAGATGAAGAAGGAGGACTTCGACATCTACGATGAGGATGA |
| | GAACCAGTCCCCTCGGAGCTTTCAGAAGAAAACCCGCCACTACTTCATCGCTGCCGTGGAGCGGCTGTGGGATTACG |
| | GGATGTCCAGCTCACCGCATGTGCTGCGGAATAGAGCGCAGTCAGGATCGGTGCCCCAGTTCAAGAAGGTCGTGTTC |
| | CAAGAGTTCACCGACGGGTCCTTCACTCAACCCCTGTACCGGGGCGAACTCAACGAACACCTGGGACTGCTTGGGCC |
| | GTATATCAGGGCAGAAGTGGAAGATAACATCATGGTCACCTTCCGCAACCAGGCCTCCCGGCCGTACAGCTTCTACT |
| | CTTCACTGATCTCCTACGAGGAAGATCAGCGGCAGGGAGCCGAGCCCCGGAAGAACTTCGTCAAGCCTAACGAAACT |
| | AAGACCTACTTTTGGAAGGTCCAGCATCACATGGCCCCGACCAAAGACGAGTTCGACTGTAAAGCCTGGGCCTACTT |
| | CTCCGATGTGGACCTGGAGAAGGACGTGCACTCGGGACTCATTGGCCCGCTCCTTGTGTGCCATACTAATACCCTGA |
| | ACCCTGCTCACGGTCGCCAAGTCACAGTGCAGGAGTTCGCCCTCTTCTTCACCATCTTCGATGAAACAAAGTCCTGG |
| | TACTTTACTGAGAACATGGAACGCAATTGCAGGGCACCCTGCAACATCCAGATGGAAGATCCCACCTTCAAGGAAAA |
| | CTACCGGTTTCATGCCATTAACGGCTACATAATGGACACGTTGCCAGGACTGGTCATGGCCCAGGACCAGAGAATCC |
| | GGTGGTATCTGCTCTCCATGGGCTCCAACGAAAACATTCACAGCATTCATTTTTCCGGCCATGTGTTCACCGTCCGG |
| | AAGAAGGAAGAGTACAAGATGGCTCTGTACAACCTCTACCCTGGAGTGTTCGAGACTGTGGAAATGCTGCCTAGCAA |
| | GGCCGGCATTTGGAGAGTGGAATGCCTGATCGGAGAGCATTTGCACGCCGGAATGTCCACCCTGTTTCTTGTGTACT |
| | CCAACAAGTGCCAGACCCCGCTGGGAATGGCCTCAGGTCATATTAGGGATTTCCAGATCACTGCTTCGGGGCAGTAC |
| | GGGCAGTGGGCACCTAAGTTGGCCCGGCTGCACTACTCTGGCTCCATCAATGCCTGGTCCACCAAGGAACCCTTCTC |
| | CTGGATTAAGGTGGACCTCCTGGCCCCAATGATTATTCACGGTATTAAGACCCAGGGTGCCCGACAGAAGTTCTCCT |
| | CACTCTACATCTCGCAATTCATCATAATGTACAGCCTGGATGGGAAGAAGTGGCAGACCTACCGGGGAAACTCCACT |
| | GGAACGCTCATGGTGTTTTTCGGCAACGTGGACTCCTCCGGCATTAAGCACAACATCTTCAACCCTCCGATCATTGC |
| | TCGGTACATCCGGCTGCACCCAACTCACTACAGCATCCGGTCCACCCTGCGGATGGAACTGATGGGTTGTGACCTGA |
| | ACTCCTGCTCCATGCCCCTTGGGATGGAATCCAAGGCCATTAGCGATGCACAGATCACCGCCTCTTCATACTTCACC |
| | AACATGTTCGCGACCTGGTCCCCGTCGAAGGCCCGCCTGCACCTCCAAGGTCGCTCCAATGCGTGGCGGCCTCAAGT |
| | GAACAACCCCAAGGAGTGGCTCCAGGTCGACTTCCAAAAGACCATGAAGGTCACCGGAGTGACCACCCAGGGCGTGA |
| | AGTCCCTGCTGACCTCTATGTACGTTAAGGAGTTCCTCATCTCCTCAAGCCAAGACGGACATCAGTGGACCCTGTTC |
| | TTCCAAAACGGAAAAGTCAAAGTATTCCAGGGCAACCAGGACTCCTTCACCCCTGTGGTCAACAGCCTGGACCCCCC |
| | ATTGCTGACCCGCTACCTCCGCATCCACCCCCAAAGCTGGGTCCACCAGATCGCACTGCGCATGGAGGTCCTTGGAT |
| | GCGAAGCCCAAGATCTGTACTAA |
| SEQ ID NO: 34 | ATGCAGATTGAGCTGTCCACTTGTTTCTTCCTGTGCCTCCTGCGCTTCTGTTTCTCCGCCACTCGCCGGTACTACCT |
| V1.0 | TGGAGCCGTGGAGCTTTCATGGGACTACATGCAGAGCGACCTGGGCGAACTCCCCGTGGATGCCAGATTCCCCCCCC |
| Expression | GCGTGCCAAAGTCCTTCCCCTTTAACACCTCCGTGGTGTACAAGAAAACCCTCTTTGTCGAGTTCACTGACCACCTG |
| cassette | TTCAACATCGCCAAGCCGCGCCCACCTTGGATGGGCCTCCTGGGACCGACCATTCAAGCTGAAGTGTACGACACCGT |
| TTP-Intron- | GGTGATCACCCTGAAGAACATGGCGTCCCACCCCGTGTCCCTGCATGCGGTCGGAGTGTCCTACTGGAAGGCCTCCG |
| BDDFVIIIco6 | AAGGAGCTGAGTACGACGACCAGACTAGCCAGCGGGAAAAGGAGGACGATAAAGTGTTCCCGGGCGGCTCGCATACT |
| XTEN (V1.0)- | TACGTGTGGCAAGTCCTGAAGGAAAACGGACCTATGGCATCCGATCCTCTGTGCCTGACTTACTCCTACCTTTCCCA |
| WPRE- | TGTGGACCTCGTGAAGGACCTGAACAGCGGGCTGATTGGTGCACTTCTCGTGTGCCGCGAAGGTTCGCTCGCTAAGG |
| bGHPolyA | AAAAGACCCAGACCCTCCATAAGTTCATCCTTTTGTTCGCTGTGTTCGATGAAGGAAAGTCATGGCATTCCGAAACT |
| | AAGAACTCGCTGATGCAGGACCGGGATGCCGCCTCAGCCCGCGCCTGGCCTAAAATGCATACAGTCAACGGATACGT |
| | GAATCGGTCACTGCCCGGGCTCATCGGTTGTCACAGAAAGTCCGTGTACTGGCACGTCATCGGCATGGGCACTACGC |
| | CTGAAGTGCACTCCATCTTCCTGGAAGGGCACACCTTCCTCGTGCGCAACCACCGCCAGGCCTCTCTGGAAATCTCC |
| | CCGATTACCTTTCTGACCGCCCAGACTCTGCTCATGGACCTGGGGCAGTTCCTTCTCTTCTGCCACATCTCCAGCCA |
| | TCAGCACGACGGAATGGAGGCCTACGTGAAGGTGGACTCATGCCCGGAAGAACCTCAGTTGCGGATGAAGAACAACG |
| | AGGAGGCCGAGGACTATGACGACGATTTGACTGACTCCGAGATGGACGTCGTGCGGTTCGATGACGACAACAGCCCC |
| | AGCTTCATCCAGATTCGCAGCGTGGCCAAGAAGCACCCCAAAACCTGGGTGCACTACATCGCGGCCGAGGAAGAAGA |
| | TTGGGACTACGCCCCGTTGGTGCTGGCACCCGATGACCGGTCGTACAAGTCCCAGTATCTGAACAATGGTCCGCAGC |
| | GGATTGGCAGAAAGTACAAGAAAGTGCGGTTCATGGCGTACACTGACGAAACGTTTAAGACCCGGGAGGCCATTCAA |
| | CATGAGAGCGGCATTCTGGGACCACTGCTGTACGGAGAGGTCGGCGATACCCTGCTCATCATCTTCAAAAACCAGGC |
| | CTCCCGGCCTTACAACATCTACCCTCACGGAATCACCGACGTGCGGCCACTCTACTCGCGGCGCCTGCCGAAGGGCG |
| | TCAAGCACCTGAAAGACTTCCCTATCCTGCCGGGCGAAATCTTCAAGTATAAGTGGACCGTCACCGTGGAGGACGGG |
| | CCCACCAAGAGCGATCCTAGGTGTCTGACTCGGTACTACTCCAGCTTCGTGAACATGGAACGGGACCTGGCATCGGG |
| | ACTCATTGGACCGCTGCTGATCTGCTACAAAGAGTCGGTGGATCAACGCGGCAACCAGATCATGTCCGACAAGCGCA |
| | ACGTGATCCTGTTCTCCGTGTTTGATGAAAACAGATCCTGGTACCTCACTGAAAACATCCAGAGGTTCCTCCCAAAC |
| | CCCGCAGGAGTGCAACTGGAGGACCCTGAGTTTCAGGCCTCGAATATCATGCACTCGATTAACGGTTACGTGTTCGA |
| | CTCGCTGCAACTGAGCGTGTGCCTCCATGAAGTCGCTTACTGGTACATTCTGTCCATCGGCGCCCAGACTGACTTCC |
| | TGAGCGTGTTCTTTTCCGGTTACACCTTTAAGCACAAGATGGTGTACGAAGATACCCTGACCCTGTTCCCTTTCTCC |
| | GGCGAAACGGTGTTCATGTCGATGGAGAACCCGGGTCTGTGGATTCTGGGATGCCACAACAGCGACTTTCGGAACCG |
| | CGGAATGACTGCCCTGCTGAAGGTGTCCTCATGCGACAAGAACACCGGAGACTACTACGAGGACTCCTACGAGGATA |
| | TCTCAGCCTACCTCCTGTCCAAGAACAACGCGATCGAGCCGCGCAGCTTCAGCCAGAACGGCGCGCCAACATCAGAG |
| | AGCGCCACCCCTGAAAGTGGTCCCGGGAGCGAGCCAGCCACATCTGGGTCGGAAACGCCAGGCACAAGTGAGTCTGC |
| | AACTCCCGAGTCCGGACCTGGCTCCGAGCCTGCCACTAGCGGCTCCGAGACTCCGGGAACTTCCGAGAGCGCTACAC |
| | CAGAAAGCGGACCCGGAACCAGTACCGAACCTAGCGAGGGCTCTGCTCCGGGCAGCCCAGCCGGCTCTCCTACATCC |
| | ACGGAGGAGGGCACTTCCGAATCCGCCACCCCGGAGTCAGGGCCAGGATCTGAACCCGCTACCTCAGGCAGTGAGAC |
| | GCCAGGAACGAGCGAGTCCGCTACACCGGAGAGTGGGCCAGGGAGCCCTGCTGGATCTCCTACGTCCACTGAGGAAG |
| | GGTCACCAGCGGGCTCGCCCACCAGCACTGAAGAAGGTGCCTCGAGCCCGCCTGTGCTGAAGAGGCACCAGCGAGAA |
| | ATTACCCGGACCACCCTCCAATCGGATCAGGAGGAAATCGACTACGACGACACCATCTCGGTGGAAATGAAGAAGGA |
| | AGATTTCGATATCTACGACGAGGACGAAAATCAGTCCCCTCGCTCATTCCAAAAGAAAACTAGACACTACTTTATCG |
| | CCGCGGTGGAAAGACTGTGGGACTATGGAATGTCATCCAGCCCTCACGTCCTTCGGAACCGGGCCCAGAGCGGATCG |
| | GTGCCTCAGTTCAAGAAAGTGGTGTTCCAGGAGTTCACCGACGGCAGCTTCACCCAGCCGCTGTACCGGGGAGAACT |
| | GAACGAACACCTGGGCCTGCTCGGTCCCTACATCCGCGCGGAAGTGGAGGATAACATCATGGTGACCTTCCGTAACC |
| | AAGCATCCAGACCTTACTCCTTCTATTCCTCCCTGATCTCATACGAGGAGGACCAGCGCCAAGGCGCCGAGCCCCGC |
| | AAGAACTTCGTCAAGCCCAACGAGACTAAGACCTACTTCTGGAAGGTCCAACACCATATGGCCCCGACCAAGGATGA |
| | GTTTGACTGCAAGGCCTGGGCCTACTTCTCCGACGTGGACCTTGAGAAGGATGTCCATTCCGGCCTGATCGGGCCGC |
| | TGCTCGTGTGTCACACCAACACCCTGAACCCAGCGCATGGACGCCAGGTCACCGTCCAGGAGTTTGCTCTGTTCTTC |
| | ACCATTTTTGACGAAACTAAGTCCTGGTACTTCACCGAGAATATGGAGCGAAACTGTAGAGCGCCCTGCAATATCCA |
| | GATGGAAGATCCGACTTTCAAGGAGAACTATAGATTCCACGCCATCAACGGGTACATCATGGATACTCTGCCGGGGC |
| | TGGTCATGGCCCAGGATCAGAGGATTCGGTGGTACTTGCTGTCAATGGGATCGAACGAAAACATTCACTCCATTCAC |
| | TTCTCCGGTCACGTGTTCACTGTGCGCAAGAAGGAGGAGTACAAGATGGCGCTGTACAATCTGTACCCCGGGGTGTT |
| | CGAAACTGTGGAGATGCTGCCGTCCAAGGCCGGCATCTGGAGAGTGGAGTGCCTGATCGGAGAGCACCTCCACGCGG |
| | GGATGTCCACCCTCTTCCTGGTGTACTCGAATAAGTGCCAGACCCCGCTGGGCATGGCCTCGGGCCACATCAGAGAC |
| | TTCCAGATCACAGCAAGCGGACAATACGGCCAATGGGCGCCGAAGCTGGCCCGCTTGCACTACTCCGGATCGATCAA |
| | CGCATGGTCCACCAAGGAACCGTTCTCGTGGATTAAGGTGGACCTCCTGGCCCCTATGATTATCCACGGAATTAAGA |
| | CCCAGGGCGCCAGGCAGAAGTTCTCCTCCCTGTACATCTCGCAATTCATCATCATGTACAGCCTGGACGGGAAGAAG |
| | TGGCAGACTTACAGGGGAAACTCCACCGGCACCCTGATGGTCTTTTTCGGCAACGTGGATTCCTCCGGCATTAAGCA |
| | CAACATCTTCAACCCACCGATCATAGCCAGATATATTAGGCTCCACCCCACTCACTACTCAATCCGCTCAACTCTTC |
| | GGATGGAACTCATGGGGTGCGACCTGAACTCCTGCTCCATGCCGTTGGGGATGGAATCAAAGGCTATTAGCGACGCC |
| | CAGATCACCGCGAGCTCCTACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGATTTCCAAAAGACCATGAAAG |
| | TGACCGGAGTCACCACCCAGGGAGTGAAGTCCCTTCTGACCTCGATGTATGTGAAGGAGTTCCTGATTAGCAGCAGC |
| | CAGGACGGGCACCAGTGGACCCTGTTCTTCCAAAACGGAAAGGTCAAGGTGTTCCAGGGGAACCAGGACTCGTTCAC |
| | ACCCGTGGTGAACTCCCTGGACCCCCCACTGCTGACGCGGTACTTGAGGATTCATCCTCAGTCCTGGGTCCATCAGA |
| | TTGCATTGCGAATGGAAGTCCTGGGCTGCGAGGCCCAGGACCTGTACTGA |
| SEQ ID NO: 35 | |
| V3.0 | TGCTCTGGTTAATAATCTCAGGAGCACAAACATTCCTGGAGGCAGGAGAAGAAATCAACATCCTGGACTTATCCTC |
| Expression | TGGGCCTCTCCCCACCTTCGATGGCCCCAGGTTAATTTTTAAAAAGCAGTCAAAAGTCCAAGTGGCCCTTGGCAGC |
| cassette | ATTTACTCTCTCTGTTTGCTCTGGTTAATAATCTCAGGAGCACAAACATTCCTGGAGGCAGGAGAAGAAATCAACA |
| Human-codon | TCCTGGACTTATCCTCTGGGCCTCTCCCCACCTTCGAACTAGCCACTAGCCTGAGGCTGGTCAAAATTGAACCTCC |
| optimized | TCCTGCTCTGAGCAGCCTGGGGGGCAGACTAAGCAGAGGGCTGTGCAGACCCACATAAAGAGCCTACTGTGTGCCA |
| A1AT-Intron- | GGCACTTCACCCGAGGCACTTCACAAGCATGCTTGGGAATGAAACTTCCAACTCTTTGGGATGCAGGTGAAACAGT |
| BDDFVIIIXTE | TCCTGGTTCAGAGAGGTGAAGCGGCCTGCCTGAGGCAGCACAGCTCTTCTTTACAGATGTGCTTCCCCACCTCTAC |
| N-WPRE- | CCTGTCTCACGGCCCCCCATGCCAGCCTGACGGTTGTGTCTGCCTCAGTCATGCTCCATTTTTCCATCGGGACCAT |
| bGHPolyA | CAAGAGGGTGTTTGTGTCTAAGGCTGACTGGGTAACTTTGGATGAGCGGTCTCTCCGCTCTGAGCCTGTTTCCTCA |
| | TCTGTCAAATGGGCTCTAACCCACTCTGATCTCCCAGGGCGGCAGTAAGTCTTCAGCATCAGGCATTTTGGGGTGA |
| | CTCAGTAAATGGTAGATCTTGCTACCAGTGGAACAGCCACTAAGGATTCTGCAGTGAGAGCAGAGGGCCAGCTAAG |
| | TGGTACTCTCCCAGAGACTGTCTGACTCACGCCACCCCCTCCACCTTGGACACAGGACGCTGTGGTTTCTGAGCCA |
| | GGTACAATGACTCCTTTCGGTAAGTGCAGTGGAAGCTGTACACTGCCCAGGCAAAGCGTCCGGGCAGCGTAGGCGG |
| | GCGACTCAGATCCCAGCCAGTGGACTTAGCCCCTGTTTGCTCCTCCGATAACTGGGGTGACCTTGGTTAATATTCA |
| | CCAGCAGCCTCCCCCGTTGCCCCTCTGGATCCACTGCTTAAATACGGACGAGGACAGGGCCCTGTCTCCTCAGCTT |
| | CAGGCACCACCACTGACCTGGGACAGGAATTCTCAGGAGCACAAACATTCCTGGAGGCAGGAGAAGAAATCAACAT |
| | CCTGGACTTATCCTCTGGGCCTCTCCCCACCGATATCTACCTGCTGATCGCCCGGCCCCTGTTCAAACATGTCCTA |
| | ATACTCTGTCGGGGCAAAGGTCGGCAGTAGTTTTCCATCTTACTCAACATCCTCCCAGTGTACGTAGGATCCTGTC |
| | TGTCTGCACATTTCGTAGAGCGAGTGTTCCGATACTCTAATCTCCCGGGGCAAAGGTCGTATTGACTTAGGTTACT |
| | TATTCTCCTTTTGTTGACTAAGTCAATAATCAGAATCAGCAGGTTTGGAGTCAGCTTGGCAGGGATCAGCAGCCTG |
| | GGTTGGAAGGAGGGGGTATAAAAGCCCCTTCACCAGGAGAAGCCGTCACACAGATCCACAAGCTCCTGCTAGAGTC |
| | GCTGCGCGCTGCCTTCGCCCCGTGCCCCGCTCCGCCGCCGCCTCGCGCCGCCCGCCCCGGCTCTGACTGACCGCGT |
| | TACTCCCACAGGTGAGCGGGCGGGACGGCCCTTCTCCTCCGGGCTGTAATTAGCGCTTGGTTTATTGACGGCTTGT |
| | TTCTTTTCTGTGGCTGCGTGAAAGCCTTGAGGGGCTCCGGGAAGGCCCTTTGTGCGGGGGGAGCGGCTCGGGGGGT |
| | GCGTGCGTGTGTGTGTGCGTGGGGAGCGCCGCGTGCGGCTCCGCGCTGCCCGGCGGCTGTGAGCGCTGCGGGCGCG |
| | GCGCGGGGCTTTGTGCGCTCCGCAGTGTGCGCGAGGGGAGCGCGGCCGGGGGCGGTGCCCCGCGGTGCGGGGGGGG |
| | CTGCGAGGGGAACAAAGGCTGCGTGCGGGGTGTGTGCGTGGGGGGGTGAGCAGGGGGTGTGGGCGCGTCGGTCGGG |
| | CTGCAACCCCCCCTGCACCCCCCTCCCCGAGTTGCTGAGCACGGCCCGGCTTCGGGTGCGGGGCTCCGTACGGGGC |
| | GTGGCGCGGGGCTCGCCGTGCCGGGCGGGGGGTGGCGGCAGGTGGGGGTGCCGGGCGGGGCGGGGCCGCCTCGGGC |
| | CGGGGAGGGCTCGGGGGAGGGGCGCGGCGGCCCCCGGAGCGCCGGCGGCTGTCGAGGCGCGGCGAGCCGCAGCCAT |
| | TGCCTTTTATGGTAATCGTGCGAGAGGGCGCAGGGACTTCCTTTGTCCCAAATCTGTGCGGAGCCGAAATCTGGGA |
| | GGCGCCGCCGCACCCCCTCTAGCGGGCGCGGGGCGAAGCGGTGCGGCGCCGGCAGGAAGGAAATGGGCGGGGAGGG |
| | CCTTCGTGCGTCGCCGCGCCGCCGTCCCCTTCTCCCTCTCCAGCCTCGGGGCTGTCCGCGGGGGGACGGCTGCCTT |
| | CGGGGGGGACGGGGCAGGGCGGGGTTCGGCTTCTGGCGTGTGACCGGCGGCTCTAGAGCCTCTGCTAACCTTGTTC |
| | TTGCCTTCTTCTTTTTCCTACAGCTCCTGGGCAACGTGCTGGTTATTGTGCTGTCTCATCATTTTGGCAAAGAATT |
| | ACTCGAGGCCACCATGCAGATTGAACTGTCCACTTGCTTCTTCCTGTGCCTCCTGCGGTTTTGCTTCTCGGCCACC |
| | CGCCGGTATTACTTAGGTGCTGTGGAACTGAGCTGGGACTACATGCAGTCCGACCTGGGAGAACTGCCGGTGGACG |
| | CGAGATTCCCACCTAGAGTCCCGAAGTCCTTCCCATTCAACACCTCCGTGGTCTACAAAAAGACCCTGTTCGTGGA |
| | GTTCACTGACCACCTTTTCAATATTGCCAAGCCGCGCCCCCCCTGGATGGGCCTGCTTGGTCCTACGATCCAAGCA |
| | GAGGTCTACGACACCGTGGTCATCACACTGAAGAACATGGCCTCACACCCCGTGTCGCTGCATGCTGTGGGAGTGT |
| | CCTACTGGAAGGCCTCAGAGGGTGCCGAATATGATGACCAGACCAGCCAGAGGGAAAAGGAGGATGACAAAGTGTT |
| | CCCGGGTGGCAGCCACACTTACGTGTGGCAAGTGCTGAAGGAAAACGGGCCTATGGCGTCGGACCCCCTATGCCTG |
| | ACCTACTCCTACCTGTCCCATGTGGACCTTGTGAAGGATCTCAACTCGGGACTGATCGGCGCCCTCTTGGTGTGCA |
| | GAGAAGGCAGCCTGGCGAAGGAAAAGACTCAGACCCTGCACAAGTTCATTCTGTTGTTTGCTGTGTTCGATGAAGG |
| | AAAGTCCTGGCACTCAGAAACCAAGAACTCGCTGATGCAGGATAGAGATGCGGCCTCGGCCAGAGCCTGGCCTAAA |
| | ATGCACACCGTCAACGGATATGTGAACAGGTCGCTCCCTGGCCTCATCGGCTGCCACAGAAAGTCCGTGTATTGGC |
| | ATGTGATCGGCATGGGTACTACTCCGGAAGTGCATAGTATCTTTCTGGAGGGCCATACCTTCTTGGTGCGCAACCA |
| | CAGACAGGCCTCGCTGGAAATCTCGCCTATCACTTTCTTGACTGCGCAGACCCTCCTTATGGACCTTGGACAGTTC |
| | CTGCTGTTCTGTCACATCAGCTCCCATCAGCATGATGGGATGGAGGCCTATGTCAAAGTGGACTCCTGCCCTGAGG |
| | AGCCACAGCTCCGGATGAAGAACAATGAGGAAGCGGAGGATTACGACGACGACCTGACTGACAGCGAAATGGACGT |
| | CGTGCGATTCGATGACGACAACAGCCCGTCCTTCATCCAAATTAGATCAGTGGCGAAGAAGCACCCCAAGACCTGG |
| | GTGCACTACATTGCCGCCGAGGAAGAGGACTGGGACTACGCGCCGCTGGTGCTGGCGCCAGACGACAGGAGCTACA |
| | AGTCCCAGTACCTCAACAACGGGCCGCAGCGCATTGGCAGGAAGTACAAGAAAGTCCGCTTCATGGCCTACACTGA |
| | TGAAACCTTCAAGACGAGGGAAGCCATCCAGCACGAGTCAGGCATCCTGGGACCGCTCCTTTACGGCGAAGTCGGG |
| | GATACCCTGCTCATCATTTTCAAGAACCAGGCATCGCGGCCCTACAACATCTACCCTCACGGGATCACAGACGTGC |
| | GCCCGCTCTACTCCCGCCGGCTGCCCAAGGGAGTGAAGCACCTGAAGGATTTTCCCATCCTGCCGGGAGAAATCTT |
| | CAAGTACAAGTGGACCGTGACTGTGGAAGATGGCCCTACCAAGTCGGACCCTCGCTGTCTGACCCGGTACTATTCC |
| | TCGTTTGTGAACATGGAGCGCGACCTGGCCTCGGGGCTGATTGGTCCGCTGCTGATCTGCTACAAGGAGTCCGTGG |
| | ACCAGCGCGGGAACCAGATCATGTCCGACAAGCGCAACGTGATCCTGTTCTCTGTCTTTGATGAAAACAGATCGTG |
| | GTACTTGACTGAGAATATCCAGCGGTTCCTGCCCAACCCAGCGGGAGTGCAACTGGAGGACCCGGAGTTCCAGGCC |
| | TCAAACATTATGCACTCTATCAACGGCTATGTGTTCGACTCGCTCCAACTGAGCGTGTGCCTGCATGAAGTGGCAT |
| | ACTGGTACATTCTGTCCATCGGAGCCCAGACCGACTTCCTGTCCGTGTTCTTCTCCGGATACACCTTCAAGCATAA |
| | GATGGTGTACGAGGACACTCTGACCCTCTTCCCATTTTCCGGAGAAACTGTGTTCATGTCAATGGAAAACCCGGGC |
| | TTGTGGATTCTGGGTTGCCATAACTCGGACTTCCGGAATAGAGGGATGACCGCCCTGCTGAAAGTGTCCAGCTGTG |
| | ACAAGAATACCGGCGATTACTACGAGGACAGCTATGAGGACATCTCCGCTTATCTGCTGTCCAAGAACAACGCCAT |
| | TGAACCCAGGTCCTTCTCCCAAAACGGTGCACCGACCTCCGAAAGCGCCACCCCAGAGTCAGGACCTGGCTCGGAA |
| | CCGGCTACCTCGGGCTCAGAGACACCGGGGACTTCCGAGTCCGCAACCCCCGAGAGTGGACCCGGATCCGAACCAG |
| | CAACCTCAGGATCAGAAACCCCGGGAACTTCGGAATCCGCCACTCCCGAGTCGGGACCAGGCACCTCCACTGAGCC |
| | TTCCGAGGGAAGCGCCCCCGGATCCCCTGCTGGATCCCCTACCAGCACTGAAGAAGGCACCTCAGAATCCGCGACC |
| | CCTGAGTCCGGCCCTGGAAGCGAACCCGCCACCTCCGGTTCCGAAACCCCTGGGACTAGCGAGAGCGCCACTCCGG |
| | AATCGGGCCCAGGAAGCCCTGCCGGATCCCCGACCAGCACCGAGGAGGGAAGCCCCGCCGGGTCACCGACTTCCAC |
| | TGAGGAGGGAGCCTCATCCCCCCCCGTGCTGAAGCGGCATCAAAGAGAGATCACCAGGACCACTCTCCAGTCCGAT |
| | CAGGAAGAAATTGACTACGACGATACTATCAGCGTGGAGATGAAGAAGGAGGACTTCGACATCTACGATGAGGATG |
| | AGAACCAGTCCCCTCGGAGCTTTCAGAAGAAAACCCGCCACTACTTCATCGCTGCCGTGGAGCGGCTGTGGGATTA |
| | CGGGATGTCCAGCTCACCGCATGTGCTGCGGAATAGAGCGCAGTCAGGATCGGTGCCCCAGTTCAAGAAGGTCGTG |
| | TTCCAAGAGTTCACCGACGGGTCCTTCACTCAACCCCTGTACCGGGGCGAACTCAACGAACACCTGGGACTGCTTG |
| | GGCCGTATATCAGGGCAGAAGTGGAAGATAACATCATGGTCACCTTCCGCAACCAGGCCTCCCGGCCGTACAGCTT |
| | CTACTCTTCACTGATCTCCTACGAGGAAGATCAGCGGCAGGGAGCCGAGCCCCGGAAGAACTTCGTCAAGCCTAAC |
| | GAAACTAAGACCTACTTTTGGAAGGTCCAGCATCACATGGCCCCGACCAAAGACGAGTTCGACTGTAAAGCCTGGG |
| | CCTACTTCTCCGATGTGGACCTGGAGAAGGACGTGCACTCGGGACTCATTGGCCCGCTCCTTGTGTGCCATACTAA |
| | TACCCTGAACCCTGCTCACGGTCGCCAAGTCACAGTGCAGGAGTTCGCCCTCTTCTTCACCATCTTCGATGAAACA |
| | AAGTCCTGGTACTTTACTGAGAACATGGAACGCAATTGCAGGGCACCCTGCAACATCCAGATGGAAGATCCCACCT |
| | TCAAGGAAAACTACCGGTTTCATGCCATTAACGGCTACATAATGGACACGTTGCCAGGACTGGTCATGGCCCAGGA |
| | CCAGAGAATCCGGTGGTATCTGCTCTCCATGGGCTCCAACGAAAACATTCACAGCATTCATTTTTCCGGCCATGTG |
| | TTCACCGTCCGGAAGAAGGAAGAGTACAAGATGGCTCTGTACAACCTCTACCCTGGAGTGTTCGAGACTGTGGAAA |
| | TGCTGCCTAGCAAGGCCGGCATTTGGAGAGTGGAATGCCTGATCGGAGAGCATTTGCACGCCGGAATGTCCACCCT |
| | GTTTCTTGTGTACTCCAACAAGTGCCAGACCCCGCTGGGAATGGCCTCAGGTCATATTAGGGATTTCCAGATCACT |
| | GCTTCGGGGCAGTACGGGCAGTGGGCACCTAAGTTGGCCCGGCTGCACTACTCTGGCTCCATCAATGCCTGGTCCA |
| | CCAAGGAACCCTTCTCCTGGATTAAGGTGGACCTCCTGGCCCCAATGATTATTCACGGTATTAAGACCCAGGGTGC |
| | CCGACAGAAGTTCTCCTCACTCTACATCTCGCAATTCATCATAATGTACAGCCTGGATGGGAAGAAGTGGCAGACC |
| | TACCGGGGAAACTCCACTGGAACGCTCATGGTGTTTTTCGGCAACGTGGACTCCTCCGGCATTAAGCACAACATCT |
| | TCAACCCTCCGATCATTGCTCGGTACATCCGGCTGCACCCAACTCACTACAGCATCCGGTCCACCCTGCGGATGGA |
| | ACTGATGGGTTGTGACCTGAACTCCTGCTCCATGCCCCTTGGGATGGAATCCAAGGCCATTAGCGATGCACAGATC |
| | ACCGCCTCTTCATACTTCACCAACATGTTCGCGACCTGGTCCCCGTCGAAGGCCCGCCTGCACCTCCAAGGTCGCT |
| | CCAATGCGTGGCGGCCTCAAGTGAACAACCCCAAGGAGTGGCTCCAGGTCGACTTCCAAAAGACCATGAAGGTCAC |
| | CGGAGTGACCACCCAGGGCGTGAAGTCCCTGCTGACCTCTATGTACGTTAAGGAGTTCCTCATCTCCTCAAGCCAA |
| | GACGGACATCAGTGGACCCTGTTCTTCCAAAACGGAAAAGTCAAAGTATTCCAGGGCAACCAGGACTCCTTCACCC |
| | CTGTGGTCAACAGCCTGGACCCCCCATTGCTGACCCGCTACCTCCGCATCCACCCCCAAAGCTGGGTCCACCAGAT |
| | CGCACTGCGCATGGAGGTCCTTGGATGCGAAGCCCAAGATCTGTACTAAGCGGCCGCTCATAATCAACCTCTGGAT |
| | TACAAAATTTGTGAAAGATTGACTGGTATTCTTAACTATGTTGCTCCTTTTACGCTATGTGGATACGCTGCTTTAA |
| | TGCCTTTGTATCATGCTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTGACGCAACCCCCACTGGT |
| | TGGGGCATTGCCACCACCTGTCAGCTCCTTTCCGGGACTTTCGCTTTCCCCCTCCCTATTGCCACGGCGGAACTCA |
| | TCGCCGCCTGCCTTGCCCGCTGCTGGACAGGGGCTCGGCTGTTGGGCACTGACAATTCCGTGGTGTTGTCGGGGAA |
| | ATCATCGTCCTTTCCTTGGCTGCTCGCCTGTGTTGCCACCTGGATTCTGCGCGGGACGTCCTTCTGCTACGTCCCT |
| | TCGGCCCTCAATCCAGCGGACCTTCCTTCCCGCGGCCTGCTGCCGGCTCTGCGGCCTCTTCCGCGTCTTCGCCTTC |
| | GCCCTCAGACGAGTCGGATCTCCCTTTGGGCCGCCTCCCCGCTGCCTAGGCGACTGTGCCTTCTAGTTGCCAGCCA |
| | TCTGTTGTTTGCCCCTCCCCCGTGCCTTCCTTGACCCTGGAAGGTGCCACTCCCACTGTCCTTTCCTAATAAAATG |
| | AGGAAATTGCATCGCATTGTCTGAGTAGGTGTCATTCTATTCTGGGGGGTGGGGTGGGGCAGGACAGCAAGGGGGA |
| | GGATTGGGAAGACAATAGCAGGCATGCTGGGGAAGACCATGGGCGCGCCAGGCCTGTCGACGCCCGGGCGGTACCG |
| | CGATCGCTCGCGACGCATAAAG |
| SEQ + D NO: 36 | ATCGATGGCCCCAGGTTAATTTTTAAAAAGCAGTCAAAAGTCCAAGTGGCCCTTGGCAGCATTTACTCTCTCTGTT |
| human | TGCTCTGGTTAATAATCTCAGGAGCACAAACATTCCTGGAGGCAGGAGAAGAAATCAACATCCTGGACTTATCCTC |
| alpha-1- | TGGGCCTCTCCCCACCTTCGATGGCCCCAGGTTAATTTTTAAAAAGCAGTCAAAAGTCCAAGTGGCCCTTGGCAGC |
| antitrypsin | ATTTACTCTCTCTGTTTGCTCTGGTTAATAATCTCAGGAGCACAAACATTCCTGGAGGCAGGAGAAGAAATCAACA |
| (A1AT) | TCCTGGACTTATCCTCTGGGCCTCTCCCCACCTTCGAACTAGCCACTAGCCTGAGGCTGGTCAAAATTGAACCTCC |
| promoter | TCCTGCTCTGAGCAGCCTGGGGGGCAGACTAAGCAGAGGGCTGTGCAGACCCACATAAAGAGCCTACTGTGTGCCA |
| | GGCACTTCACCCGAGGCACTTCACAAGCATGCTTGGGAATGAAACTTCCAACTCTTTGGGATGCAGGTGAAACAGT |
| | TCCTGGTTCAGAGAGGTGAAGCGGCCTGCCTGAGGCAGCACAGCTCTTCTTTACAGATGTGCTTCCCCACCTCTAC |
| | CCTGTCTCACGGCCCCCCATGCCAGCCTGACGGTTGTGTCTGCCTCAGTCATGCTCCATTTTTCCATCGGGACCAT |
| | CAAGAGGGTGTTTGTGTCTAAGGCTGACTGGGTAACTTTGGATGAGCGGTCTCTCCGCTCTGAGCCTGTTTCCTCA |
| | TCTGTCAAATGGGCTCTAACCCACTCTGATCTCCCAGGGCGGCAGTAAGTCTTCAGCATCAGGCATTTTGGGGTGA |
| | CTCAGTAAATGGTAGATCTTGCTACCAGTGGAACAGCCACTAAGGATTCTGCAGTGAGAGCAGAGGGCCAGCTAAG |
| | TGGTACTCTCCCAGAGACTGTCTGACTCACGCCACCCCCTCCACCTTGGACACAGGACGCTGTGGTTTCTGAGCCA |
| | GGTACAATGACTCCTTTCGGTAAGTGCAGTGGAAGCTGTACACTGCCCAGGCAAAGCGTCCGGGCAGCGTAGGCGG |
| | GCGACTCAGATCCCAGCCAGTGGACTTAGCCCCTGTTTGCTCCTCCGATAACTGGGGTGACCTTGGTTAATATTCA |
| | CCAGCAGCCTCCCCCGTTGCCCCTCTGGATCCACTGCTTAAATACGGACGAGGACAGGGCCCTGTCTCCTCAGCTT |
| | CAGGCACCACCACTGACCTGGGACAG |

1. A nucleic acid molecule comprising a first inverted terminal repeat (ITR) and a second ITR flanking a genetic cassette comprising a heterologous polynucleotide sequence, wherein:
the first ITR comprises a polynucleotide sequence at least about 75% identical to SEQ ID NO: 1, and the second ITR comprises a polynucleotide sequence at least about 75% identical to SEQ ID NO: 2.

2-6. (canceled)

7. The nucleic acid molecule of claim 1, further comprising:
a. a promoter optionally wherein the promoter is a tissue specific promoter;
b. an intronic sequence optionally wherein the intronic sequence is synthetic intronic sequence,
c. a post-transcriptional regulatory element
d. an enhancer sequence; and/or
e. a 3′UTR poly(A) tail sequence.

8-23. (canceled)

24. The nucleic acid molecule of claim 1, wherein the nucleic acid molecule comprises from 5′ to 3′: the first ITR, the genetic cassette, and the second ITR, wherein the genetic cassette comprises a tissue-specific promoter sequence, an intronic sequence, the heterologous polynucleotide sequence, a post-transcriptional regulatory element, and a 3′UTR poly(A) tail sequence.

25. The nucleic acid molecule of claim 24, wherein the genetic cassette comprises from 5′ to 3′: a tissue-specific promoter sequence, an intronic sequence, the heterologous polynucleotide sequence, a post-transcriptional regulatory element, and a 3′UTR poly(A) tail sequence.

26. The nucleic acid molecule of claim 25, wherein the genetic cassette comprises the nucleotide sequence of SEQ ID NO: 3, 9, 14, 33, or 35.

27. (canceled)

28. (canceled)

29. The nucleic acid molecule of claim 1, wherein the heterologous polynucleotide sequence encodes a therapeutic protein, preferably wherein the heterologous polynucleotide encodes a clotting factor, more preferably wherein the clotting factor is factor IX.

30-36. (canceled)

37. The nucleic acid molecule of claim 1, wherein the heterologous polynucleotide sequence encodes a microRNA (miRNA).

38. (canceled)

39. (canceled)

40. The nucleic acid molecule of claim 1, wherein the heterologous polynucleotide sequence is codon optimized, preferably wherein the heterologous sequence is codon optimized for expression in a human.

41. (canceled)

42. The nucleic acid molecule of claim 1, wherein the nucleic acid molecule is formulated with a delivery agent.

43. The nucleic acid molecule of claim 42, wherein the delivery agent comprises a lipid nanoparticle (LNP), liposomes, non-lipid polymeric molecules, endosomes, or any combination thereof.

44-48. (canceled)

49. A vector comprising the nucleic acid molecule of claim 1.

50. A host cell comprising the nucleic acid molecule of claim 1.

51. (canceled)

52. A pharmaceutical composition comprising the nucleic acid of claim 1.

53-55. (canceled)

56. A baculovirus system for production of the nucleic acid molecule claim 1.

57. (canceled)

58. The baculovirus system of claim 56, further comprising a recombinant bacmid, wherein the recombinant bacmid comprises a variant VP80 gene, such that the bacmid exhibits reduced expression of its encoded protein.

59-61. (canceled)

62. A method of treating a bleeding disorder comprising:
administering a nucleic acid molecule to a subject in need thereof,
wherein the nucleic acid molecule comprises the nucleotide sequence of SEQ ID NO: 3.

63. The method claim 62, wherein the disorder is hemophilia A.

64-69. (canceled)

70. A recombinant bacmid comprising:
a sequence encoding an HBoV1 Rep, wherein the inserted HBoV1 Rep sequence disrupts the reading frame of a reporter gene or functional portion thereof; and/or
a heterologous sequence comprising a nucleic acid molecule comprising the nucleotide sequence of SEQ ID NO: 3, 9, 14, 33, or 35.

71. (canceled)

72. A stable cell line comprising the genetic cassette of claim 26, wherein the nucleic acid sequence is stably integrated in the genome of the stable cell line.

73. (canceled)

74. A method of generating a closed ended DNA (ceDNA) molecule comprising infecting an insect cell with a recombinant baculovirus comprising the set of recombinant bacmids of claim 70.

75. (canceled)
